(19) United States
(12) Patent Application Publication
(10) Pub. No.: US 2011/0159486 A1
(43) Pub. Date: Jun. 30, 2011

(54) CELL CYCLE SWITCH 52(CCS52) AND METHODS FOR INCREASING YIELD

(75) Inventors: William J. Gordon-Kamm, Urbandale, IA (US); Carlos M. La Rota, Johnston, IA (US)

(73) Assignee: Pioneer Hi-Bred International, Inc., Johnston, IA (US)

(21) Appl. No.: 12/976,531

(22) Filed: Dec. 22, 2010

Related U.S. Application Data
(60) Provisional application No. 61/291,722, filed on Dec. 31, 2009.

(51) Int. Cl. C12Q 1/68; C07H 21/04; C12N 15/82; A01H 5/00; A01H 5/10; C07K 14/415; C12N 5/10

(52) U.S. Cl. 435/6.1; 536/23.6; 435/320.1; 800/298; 800/295; 530/350; 435/419; 435/412; 435/415; 435/416

(57) ABSTRACT

Methods and compositions for modulating plant yield are provided. Methods include employing cell cycle switch 52 (ccs52). The ccs52 sequences are used in a variety of methods including modulating plant biomass, growth, or both. Transformed plants, plant cell, tissues, seed, and expression vectors are also provided.

### CROSS-REFERENCE TO RELATED APPLICATIONS

This Application claims the benefit of U.S. Provisional Application 61/291,722 filed Dec. 31, 2009, herein incorporated by reference it its entirety.

### FIELD OF THE INVENTION

The invention relates to the field of the genetic manipulation of plants; in particular, the modulation of gene activity and development in plants.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 399644SEQLIST.txt, created on Dec. 21, 2010, and having a size of 174 kilobytes and is filed concurrently with the specification. The sequence listing contained in this ASCII formatted document is part of the specification and is herein incorporated by reference in its entirety.

### BACKGROUND OF THE INVENTION

Given the ever-increasing world population, it remains a major goal of agricultural research to improve the efficiency of agriculture. Conventional means for crop and horticulture improvements utilize selective breeding techniques to identify plants having desirable characteristics. However, such selective breeding techniques have several drawbacks, namely that these techniques are typically labor intensive and result in plants that often contain heterogenous genetic complements that may not always result in the desirable trait being passed on from parent plants. In contrast, advances in molecular biology have allowed mankind to more precisely manipulate the germplasm of plants. Genetic engineering of plants entails the isolation and manipulation of genetic material (typically in the form of DNA or RNA) and the subsequent introduction of that genetic material into a plant. Such technology has led to the development of plants having various improved economic, agronomic or horticultural traits. A trait of particular economic interest is high yield.

The ability to improve one or more plant growth characteristics would have many applications in areas such as crop enhancement, plant breeding, production of ornamental plants, arboriculture, horticulture, forestry, production of algae or plants (for use as bioreactors for example, for the production of pharmaceuticals, such as antibodies or vaccines, or for the bioconversion of organic waste, or for use as fuel, in the case of high-yielding algae and plants).

CCS52 (Cell Cycle Switch 52, also referred to as FZR, or Fizzy-Related in certain species) belongs to a small group of proteins containing several WD repeat motifs and is the plant homologue of animal APC activators involved in mitotic cyclin degradation (WO99/64451). In Cebolla et al. (EMBO J., (1999) 18: 4476-84), the isolation of ccs52 clones from Medicago sativa root nodules was reported and ccs52 was described to be part of a small gene family that appears to be conserved in plants. Furthermore, the functional domains and regulation mechanisms of ccs52 proteins have been described in detail by Tarayre et al. (The Plant Cell, (2004) 16:422-34). Suppression of ccs52 reduced the ploidy level in cells within developing nodules, and overexpression of the Medicago gene in yeast results in cell cycle arrest, endoreduplication, and cell enlargement. Loss of function ccs52 alleles resulted in fewer endocycles and smaller plants in Arabidopsis, while constitutive overexpression of ccs52 (FZR2 led to dwarfing, anthocycnin accumulation and increased ploidy levels in trichomes (Larson-Rabin et al. (2009) Plant Physiol 149:874-884). Thus, ccs52's are involved in a variety of aspects of cell replication and development.

### SUMMARY OF THE INVENTION

Generally, it is the object of the present invention to provide polynucleotides and polypeptides relating to ccs52. It is an object of the present invention to provide transgenic plants comprising the polynucleotides and polypeptides of the present invention. Additionally, it is an object of the present invention to provide methods of modulating, in a plant cell or in a transgenic plant, the expression of the ccs52 polynucleotides and polypeptides, using known ccs52 polynucleotides and polypeptides or ccs52 polynucleotides and polypeptides of the present invention. Yet another object of the present invention is to provide methods of increasing yield in a plant. In another aspect, yield is increased while maintaining fertility of the plant or progeny thereof. Accordingly, it is an object of the present invention to provide transgenic plants expressing known ccs52 polynucleotides and polypeptides or ccs52 polynucleotides and polypeptides of the present invention in a plant cell. The ccs52 is not expressed or effectively expressed in a germline plant cell or a plant cell that contributes to the germline. In another aspect, the ccs52 polynucleotide and polypeptide is not expressed or effectively expressed in meristematic plant cells. In one example, the ccs52 sequence is expressed in a plant cell that is committed to becoming differentiated plant cell having chlorophyll, a differentiated plant cell having chlorophyll or both.

In one aspect, the present invention relates to an isolated ccs52 polynucleotide that encodes the polypeptide of SEQ ID NO: 2, 4, 6, 8, 10 or 12; a polynucleotide having the sequence of SEQ ID NO: 1, 3, 5, 7, 9, or 11; a polynucleotide having at least 30 nucleotides in length which hybridizes under stringent conditions to any of the former polynucleotides. In another aspect, the present invention includes a polynucleotide having at least 60%, 70%, 80%, 85%, 90%, 95% or 100% sequence identity to SEQ ID NO: 1, 3, 5, 7, 9, or 11. Provided herein in another aspect of the invention are isolated polynucleotides degenerate as a result of the genetic code for any of the ccs52's of the present invention. In another aspect, an isolated polynucleotide is complementary to a polynucleotide of any one of the ccs52's of the present invention. In another aspect, the present invention relates to an isolated polynucleotide that encodes a ccs52 polypeptide that increases yield in a plant while maintaining fertility.

In yet another aspect, the present invention relates to a transgenic plant including a recombinant expression cassette comprising a plant promoter operably linked to any of the isolated polynucleotides of the present invention or a known isolated polynucleotide encoding a ccs52 polypeptide. The plant promoter preferentially expresses the polynucleotide in non-germline plant cells, for example, a plant cell that expresses photosynthetic genes. In some cases, the plant cell is a plant cell committed to becoming differentiated plant cell having chlorophyll, a differentiated plant cell having chlorophyll or both. In another aspect, the ccs52 polynucleotide and polypeptide is not expressed or effectively expressed in a meristematic plant cell.

In one aspect, the plant is a fertile plant. The present invention also provides for transgenic seed from the transgenic plant. In another aspect, the present invention is directed to a host cell transfected with the recombinant expression cassette comprising a plant promoter operably linked to any of the isolated polynucleotides of the present invention or known ccs52. In one aspect, the host cell is a dicot or monocot cell.

In a further aspect, the present invention relates to an isolated polypeptide having an amino acid sequence having at least 60%, 70%, 80%, 85%, 90%, 95% or 100% sequence identity to the amino acid sequence set forth in SEQ ID NO: 2, 4, 6, 8, 10 or 12 and ccs52 activity. In yet another aspect, the present invention relates to a transgenic plant comprising a recombinant expression cassette comprising a plant promoter operably linked to an isolated polynucleotide encoding a polypeptide that has an amino acid sequence that has at least 60%, 70%, 80%, 85%, 90%, 95% or 100% sequence identity to the amino acid sequence set forth in SEQ ID NO: 2, 4, 6, 8, 10 or 12 and has ccs52 activity or a known isolated polynucleotide encoding a ccs52 polypeptide having ccs52 activity. The plant promoter preferentially expresses the polynucleotide in non-germline plant cells, for example, plant cells that express photosynthetic genes. In some cases, the plant cell is a plant cell such as plant cells committed to becoming differentiated plant cells having chlorophyll, differentiated plant cells having chlorophyll or combinations thereof. The plant promoter preferentially expresses the polynucleotide in non-meristematic plant cells. The present invention also provides for transgenic seed from the transgenic plant. In another aspect, the present invention is directed to a host cell transfected with the recombinant expression cassette comprising a plant promoter operably linked to any of the isolated polynucleotides encoding polypeptides of the present invention or known isolated polynucleotide encoding a ccs52 polypeptide having ccs52 activity. The plant promoter preferentially expresses the polynucleotide in plant cells committed to becoming differentiated plant cells having chlorophyll, differentiated plant cells having chlorophyll or both.

In a further aspect, the present invention relates to a method of modulating the level of ccs52 protein in a photosynthetic plant cell that is committed to becoming differentiated plant cell having chlorophyll, a differentiated plant cell having chlorophyll or both. In one aspect, the method includes transforming a plant cell with a ccs52 polynucleotide operably linked to a promoter. The method may include stably transforming the plant cell. The plant promoter preferentially expresses the polynucleotide in non-germline plant cells or non-meristematic plant cells, such as plant cells that express photosynthetic genes. In some cases, the plant cell is a plant cell committed to becoming differentiated plant cells having chlorophyll, differentiated plant cells having chlorophyll or combinations thereof. The polynucleotide may be in sense or antisense orientation. The method further includes expressing the polynucleotide for an amount of time sufficient to modulate the ccs52 protein in the plant cell, for example, a plant cell that is committed to becoming differentiated plant cell having chlorophyll or in a differentiated plant cell having chlorophyll. The method includes regenerating the transformed plant cell into a transformed plant that expresses the ccs52 polynucleotide in an amount sufficient to modulate the level of ccs52 protein in non-germline or non-meristematic plant cells, such as plant cells that express photosynthetic genes. In some cases, the plant cells are plant cells committed to becoming differentiated plant cells having chlorophyll, differentiated plant cells having chlorophyll or combinations thereof.

In another aspect, the present invention relates to a method of increasing yield in a plant. In one aspect, the method includes introducing into plant cells a construct comprising a known polynucleotide encoding a ccs52 or a polynucleotide encoding a ccs52 of the present invention in plant cells to yield transformed plant cells. The polynucleotide may be operably linked to a promoter that preferentially expresses the polynucleotide in non-germline plant cells, such as plant cells that express photosynthetic genes. In some cases, the plant cells are plant cells committed to becoming differentiated plant cells having chlorophyll, differentiated plant cells having chlorophyll or combinations thereof. In another aspect, the plant promoter preferentially expresses the polynucleotide in non-meristematic plant cells. The transformed plant cells are regenerated into a transgenic plant. The ccs52 is expressed in at least some of the non-germline plant cells, such as the committed and/or differentiated plant cells having chlorophyll, of the transgenic plant at levels sufficient to increase yield. In another aspect, the plant is fertile. In another aspect, progeny of the plant thereof are also fertile.

Other objects, features, advantages and aspects of the present invention will become apparent to those of skill from the following description.

The following embodiments are encompassed by the present invention:
1. An isolated or recombinant nucleic acid comprising a polynucleotide sequence selected from the group consisting of:
(a) a polynucleotide that encodes the polypeptide of SEQ ID NO: 2, 4, 6, 8, 10 or 12;
(b) a polynucleotide comprising the sequence set forth in SEQ ID NO: 1, 3, 5, 7, 9, or 11;
(c) a polynucleotide comprising at least 300 nucleotides in length which hybridizes under stringent conditions to a polynucleotide of (a) or (b), wherein the conditions include hybridization in 40 to 45% formamide, 1 M NaCl, 1% SDS at 37° C. and a wash in 0.5× to 1×SSC at 55° C. to 60° C.; and
(d) a polynucleotide having at least 85%, 90%, or 95% sequence identity to SEQ ID NO: 1, 3, 5, 7, 9, or 11, wherein the % sequence identity is based on the entire encoding region and is determined by BLAST 2.0 under default parameters wherein the polynucleotide encodes a polypeptide having cell cycle switch 52 activity; and
(e) a polynucleotide encoding a polypeptide that is at least 85%, 90%, or 95% identical to a polypeptide comprising the sequence set forth in SEQ ID NO: 2, 4, 6, 8, 10 or 12, wherein the encoded polypeptide has ccs52 activity;
(f) a polynucleotide encoding a polypeptide fragment of at least about 200 amino acid residues, wherein the encoded polypeptide fragment has ccs52 activity;
(g) an isolated polynucleotide degenerate from any of (a) to (f) as a result of the genetic code; and
(h) a polynucleotide complementary to a polynucleotide of any one of (a) to (g).
2. An isolated or recombinant nucleic acid according to embodiment 1 wherein said polynucleotide encodes a cell cycle switch 52 polypeptide that confers increased yield in a plant.
3. A vector comprising at least one polynucleotide of embodiment 1.
4. An expression cassette comprising at least one polynucleotide of embodiment 1 operably linked to a promoter, wherein the polynucleotide is in sense orientation.
5. The expression cassette of embodiment 4 wherein the promoter preferentially expresses the polynucleotide in a plant cell committed to becoming a differentiated plant cell having chlorophyll or differentiated plant cell having chlorophyll or both.
6. A host cell into which is introduced at least one expression cassette of embodiment 4.
7. The host cell of embodiment 6 that is a plant cell.
8. A transgenic plant comprising at least one expression cassette of embodiment 4.
9. The transgenic plant of embodiment 8, wherein the plant is selected from the group consisting of: corn, soybean, sunflower, sorghum, canola, wheat, alfalfa, cotton, rice, barley and millet.
10. A seed from the transgenic plant of embodiment 9.
11. An isolated polypeptide selected from the group consisting of:
a) an isolated polypeptide encoded by the polynucleotide of SEQ ID NO: 1, 3, 5, 7, 9, or 11;
b) an isolated polypeptide comprising SEQ ID NO: 2, 4, 6, 8, 10 or 12, said polypeptide having cell cycle switch 52 activity;
c) a polypeptide that is at least 85%, 90%, or 95% identical to the amino acid sequence of SEQ ID NO: 2, 4, 6, 8, 10 or 12, said polypeptide having cell cycle switch 52 activity;
d) a polypeptide that is encoded by a nucleic acid molecule comprising a nucleotide sequence that is at least 85%, 90% or 95% identical to SEQ ID NO: 1, 3, 5, 7, 9, or 11, or a complement thereof, said polypeptide having cell cycle switch 52 activity;
e) a polypeptide that is encoded by a nucleic acid molecule that hybridizes with a nucleic acid probe consisting of the nucleotide sequence of SEQ ID NO: 1, 3, 5, 7, 9, or 11, or a complement thereof following at least one wash in 0.2×SSC at 55° C. for 20 minutes, said polypeptide having cell cycle switch 52 activity; and
f) a fragment comprising at least 200 consecutive amino acids of SEQ ID NO: 2, 4, 6, 8, 10 or 12, said polypeptide having cell cycle switch 52 activity.
12. A recombinant expression cassette comprising a polynucleotide operably linked to a promoter, wherein the polynucleotide encodes the polypeptide of embodiment 11.
13. The recombinant expression cassette of embodiment 12 comprising a polynucleotide operably linked to a promoter, wherein the promoter preferentially expresses the polynucleotide in a plant cell committed to becoming a differentiated plant cell having chlorophyll or a differentiated plant cell having chlorophyll or both.
14. A transformed host cell comprising the isolated polypeptide of embodiment 11.
15. The host cell of embodiment 14, wherein the host cell is a transformed plant cell.
16. The plant cell of embodiment 15, wherein the plant cell is selected from the group consisting of sorghum, maize, rice, wheat, soybean, sunflower, canola, alfalfa, barley, and millet.
17. A transformed plant regenerated from the plant cell of embodiment 16.
18. A transformed seed of the plant of embodiment 17.
19. The isolated polypeptide of embodiment 11 wherein the expression of cell cycle switch 52 in a plant cell results in increased ploidy in the plant cell as compared to a control plant cell, wherein the control plant that does not contain the polynucleotide encoding the ccs52.
20. The isolated polypeptide of embodiment 11 wherein the expression of cell cycle switch 52 in a plant results in increased yield in the plant as compared to a control plant, wherein the control plant that does not contain the polynucleotide encoding the ccs52.
21. A method of modulating the level of cell cycle switch 52 protein in a plant cell, comprising:
(a) transforming a plant cell with a cell cycle switch 52 polynucleotide operably linked to a promoter, wherein the polynucleotide is in sense orientation, and wherein the polynucleotide sequence is selected from the group consisting of:
   (1) a polynucleotide that encodes the polypeptide of SEQ ID NO: 2, 4, 6, 8, 10 or 12;
   (2) a polynucleotide comprising the sequence set forth in SEQ ID NO: 1, 3, 5, 7, 9, or 11;
   (3) a polynucleotide comprising at least 300 nucleotides in length which hybridizes under stringent conditions to a polynucleotide of (a) or (b), wherein the conditions include hybridization in 40 to 45% formamide, 1 M NaCl, 1% SDS at 37° C. and a wash in 0.5× to 1×SSC at 55 to 60° C.; and
   (4) a polynucleotide having at least 85%, 90%, or 95% sequence identity to SEQ ID NO: 1, 3, 5, 7, 9, or 11, wherein the % sequence identity is based on the entire encoding region and is determined by BLAST 2.0 under default parameters wherein the polynucleotide encodes a polypeptide having cell cycle switch 52 activity; and
   (5) a polynucleotide encoding a polypeptide that is at least 85%, 90%, or 95% identical to a polypeptide comprising the sequence set forth in SEQ ID NO: 2, 4, 6, 8, 10 or 12, wherein the encoded polypeptide has ccs52 activity;
   (6) a polynucleotide encoding a polypeptide fragment of at least about 200 amino acid residues, wherein the encoded polypeptide fragment has cell cycle switch 52 activity;
   (7) an isolated polynucleotide degenerate from any of (1) to (6) as a result of the genetic code; and
   (8) a polynucleotide complementary to a polynucleotide of any one of (1) to (7); and
(b) expressing the polynucleotide in a plant cell committed to becoming a differentiated plant cell having chlorophyll or a differentiated plant cell having chlorophyll or combinations thereof for a time sufficient to modulate the cell cycle switch 52 protein in the plant cell.
22. The method of embodiment 21, wherein the plant is corn, soybean, sunflower, sorghum, canola, wheat, alfalfa, cotton, rice, barley or millet.
23. The method of embodiment 21, wherein cell cycle switch 52 protein is increased as compared to a control plant cell, wherein the control plant cell does not contain the polynucleotide encoding the cell cycle switch 52.
24. The method of embodiment 21, wherein the promoter preferentially expresses the polynucleotide in a plant cell committed to becoming a differentiated plant cell having chlorophyll or a differentiated plant cell having chlorophyll or combinations thereof and wherein the promoter does not effectively express the polynucleotide in germline plant cells or in plant cells that will contribute to the germline of the plant.
25. The method of embodiment 21, wherein the plant cell is stably transformed with the cell cycle switch 52 polynucleotide.
26. The method of embodiment 21, wherein the expression of cell cycle switch 52 in a plant cell committed to becoming a differentiated plant cell having chlorophyll or a differentiated plant cell having chlorophyll results in a fertile plant as compared to a control plant cell, wherein the control plant has expression of cell cycle switch 52 in germline plant cells or in plant cells that will contribute to the germline.
27. The method of embodiment 21, wherein the expression of cell cycle switch 52 in a plant cell results in increased ploidy in the plant cell as compared to a control plant cell, wherein the control plant that does not contain the polynucleotide encoding the cell cycle switch 52.
28. The method of embodiment 21, wherein the expression of cell cycle switch 52 in a plant results in increased yield in the plant as compared to a control plant, wherein the control plant that does not contain the polynucleotide encoding the cell cycle switch 52.
29. A method for increasing yield in a plant, said method comprising the steps of:
(a) introducing into plant cells a construct comprising a polynucleotide encoding a cell cycle switch 52, wherein said cell cycle switch 52 polynucleotide is operably linked to a promoter that does not effectively express the cell cycle switch 52 polynucleotide in plant germline cells but wherein the promoter preferentially expresses the cell cycle switch 52 polynucleotide in non-germline plant cells to yield transformed plant cells or combinations thereof,
(b) regenerating a transgenic plant from said transformed plant cells, wherein said cell cycle switch 52 is expressed in the plant cells at levels sufficient to increase yield in said transgenic plant.
30. The plant of embodiment 29, wherein said promoter comprises a tissue-preferred, constitutive, or inducible promoter.
31. The method of embodiment 29, wherein the promoter preferentially expresses the polynucleotide in plant cells committed to becoming differentiated plant cells having chlorophyll or differentiated plant cells having chlorophyll or combinations thereof.
32. The method of embodiment 33, wherein the promoter is a leaf-preferred promoter.
33. The method of embodiment 33, wherein the promoter is promoter of the chlorophyll a/b binding protein.
34. The method of embodiment 29, wherein the cell cycle switch 52 is a dicot polynucleotide.
35. The method of embodiment 29, wherein the cell cycle switch 52 is a monocot polynucleotide.
36. The method of embodiment 29, wherein the cell cycle switch 52 polynucleotide encoding the cell cycle switch 52 protein is selected from the group consisting of:
(a) a polynucleotide that encodes the polypeptide of SEQ ID NO: 2, 4, 6, 8, 10 or 12;
(b) a polynucleotide comprising the sequence set forth in SEQ ID NO: 1, 3, 5, 7, 9, or 11;
(c) a polynucleotide comprising at least 300 nucleotides in length which hybridizes under stringent conditions to a polynucleotide of (a) or (b), wherein the conditions include hybridization in 40 to 45% formamide, 1 M NaCl, 1% SDS at 37° C. and a wash in 0.5× to 1×SSC at 55 to 60° C.; and
(d) a polynucleotide having at least 85%, 90%, or 95% sequence identity to SEQ ID NO: 1, 3, 5, 7, 9, or 11, wherein the % sequence identity is based on the entire encoding region and is determined by BLAST 2.0 under default parameters wherein the polynucleotide encodes a polypeptide having cell cycle switch 52 activity; and
(e) a polynucleotide encoding a polypeptide that is at least 85%, 90%, or 95% identical to a polypeptide comprising the sequence set forth in SEQ ID NO: 2, 4, 6, 8, 10 or 12, wherein the encoded polypeptide has cell cycle switch 52 activity;
(f) a polynucleotide encoding a polypeptide fragment of at least about 25 amino acid residues, wherein the encoded polypeptide fragment has cell cycle switch 52 activity;
(g) an isolated polynucleotide degenerate from any of (a) to (f) as a result of the genetic code; and
(h) a polynucleotide complementary to a polynucleotide of any one of (a) to (g).
37. The method of embodiment 29, wherein increased yield comprises increased ear size, increased seed set, increased chlorophyll content, increased level of photosynthetic machinery, increase cell size of said differentiated plant cells having cholorphyll, or increased overall source levels of photosynthate.
38. The method of embodiment 29, wherein the yield of the plant is compared to a control plant, wherein the control plant does not contain the polynucleotide encoding the cell cycle switch 52 polypeptide.
39. The method of embodiment 29, wherein the plant is fertile.
40. The method of embodiment 31, wherein the expression of cell cycle switch 52 in the plant cell committed to becoming a differentiated plant cell having chlorophyll or differentiated plant cell having chlorophyll results in increased ploidy in the plant cell as compared to a control plant cell, wherein the control plant that does not contain the polynucleotide encoding the cell cycle switch 52.
41. The method of embodiment 40, determining the ploidy of the committed or differentiated plant cells of the plant.
42. The method of embodiment 41, determining duplication of the genome by isolating nuclei from leaf cells of the plant; and
   determining ploidy of the nuclei in the cells.
43. The method of embodiment 29, wherein the construct further comprises a second polynucleotide, wherein said second polynucleotide encodes a cell cycle G1-S transition stimulating gene, and wherein said polynucleotide is operably linked to a promoter functional in plant cells.
44. The method of embodiment 43, wherein the second polynucleotide is RepA, CycD or E2F.
45. The method of embodiment 43, wherein the promoter is a promoter that drives expression in a plant cell committed to becoming a differentiated plant cell having chlorophyll or a differentiated plant cell having chlorophyll.
46. The method of embodiment 29, wherein the plant is a dicotyledonous plant.
47. The method of embodiment 46, wherein said dicot is selected from the group consisting of soybean, Brassica spp., sunflower, safflower, alfalfa, cotton, tomato, and Arabidopsis.
48. The method of embodiment 29, wherein the plant is a monocotyledonous plant.
50. The method of embodiment 48, wherein said monocot is selected from the group consisting of maize, sorghum, wheat, rice, barley, rye, and millet.
51. The method of embodiment 29, wherein the plant is stably transformed with the cell cycle switch 52 polynucleotide.
52. An expression cassette comprising at least one cell cycle switch 52 polynucleotide operably linked to a promoter, wherein the polynucleotide is in sense orientation, and wherein the promoter does not effectively express the cell cycle switch 52 polynucleotide in plant germline cells but wherein the promoter preferentially expresses the cell cycle switch 52 polynucleotide in non-germline plant cells.
53. The expression cassette of embodiment 52, wherein said promoter comprises a tissue-preferred, constitutive, or inducible promoter.
54. The expression cassette of embodiment 52, wherein the promoter preferentially expresses the polynucleotide in plant cells committed to becoming differentiated plant cells having chlorophyll or differentiated plant cells having chlorophyll or combinations thereof.
55. The expression cassette of embodiment 52, wherein the promoter is a leaf-preferred promoter.
56. The expression cassette of embodiment 52, wherein the promoter is promoter of the chlorophyll a/b binding protein.
57. The expression cassette of embodiment 52, wherein the cell cycle switch 52 is a dicot polynucleotide or monocot polynucleotide.
58. The expression cassette of embodiment 52, wherein the cell cycle switch 52 is a vertebrate polynucleotide or invertebrate polynucleotide.
59. The expression cassette of embodiment 52, wherein the cell cycle switch 52 polynucleotide encoding the cell cycle switch 52 protein is selected from the group consisting of:
(a) a polynucleotide that encodes the polypeptide of SEQ ID NO: 2, 4, 6, 8, 10 or 12;
(b) a polynucleotide comprising the sequence set forth in SEQ ID NO: 1, 3, 5, 7, 9, or 11;
(c) a polynucleotide comprising at least 300 nucleotides in length which hybridizes under stringent conditions to a polynucleotide of (a) or (b), wherein the conditions include hybridization in 40 to 45% formamide, 1 M NaCl, 1% SDS at 37° C. and a wash in 0.5× to 1×SSC at 55 to 60° C.; and
(d) a polynucleotide having at least 85%, 90%, or 95% sequence identity to SEQ ID NO: 1, 3, 5, 7, 9, or 11, wherein the % sequence identity is based on the entire encoding region and is determined by BLAST 2.0 under default parameters wherein the polynucleotide encodes a polypeptide having cell cycle switch 52 activity; and
(e) a polynucleotide encoding a polypeptide that is at least 85%, 90%, or 95% identical to a polypeptide comprising the sequence set forth in SEQ ID NO: 2, 4, 6, 8, 10 or 12, wherein the encoded polypeptide has cell cycle switch 52 activity;
(f) a polynucleotide encoding a polypeptide fragment of at least about 25 amino acid residues, wherein the encoded polypeptide fragment has cell cycle switch 52 activity;
(g) an isolated polynucleotide degenerate from any of (a) to (f) as a result of the genetic code; and
(h) a polynucleotide complementary to a polynucleotide of any one of (a) to (g).
60. A host cell into which is introduced at least one expression cassette of embodiment 52.
61. The host cell of embodiment 60 that is a plant cell.
62. A transgenic plant comprising at least one expression cassette of embodiment 52.
63. The transgenic plant of embodiment 62, wherein the plant is a dicot.
64. The transgenic plant of embodiment 62, wherein the dicot is selected from the group consisting of soybean, Brassica spp., sunflower, safflower, alfalfa, cotton, tomato, and Arabidopsis.
65. The transgenic plant of embodiment 62, wherein the plant is a monocot.
66. The transgenic plant of embodiment 62, wherein the monocot is selected from the group consisting of maize, sorghum, wheat, rice, barley, rye, and millet.
67. A seed from the transgenic plant of embodiment 62.
68. A transformed host cell comprising a polypeptide encoded by the polynucleotide in the expression cassette of embodiment 52.
69. The host cell of embodiment 68, wherein the host cell is a transformed plant cell.
70. The plant cell of embodiment 69, wherein the plant cell is selected from the group consisting of sorghum, maize, rice, wheat, soybean, sunflower, canola, alfalfa, barley, and millet.

### BRIEF DESCRIPTION OF THE DRAWINGS

The invention can be more fully understood from the following detailed description and the accompanying figures and sequences and sequence listing which form a part of this application.

FIG. 1 shows a phylogenetic tree constructed from the maize (ZmFZR-PHI=SEQ ID NO:2); rice (OsFZR) (SEQ ID NO:30), alfalfa (Ms Ccs52) (SEQ ID NO:28), Arabidopsis (AtFZR 3) (SEQ ID NO:18), human (HsFZR1) (SEQ ID NO:24), Drosophila (DmFZY) (SEQ ID NO:20), mouse (MmFZR1) (SEQ ID NO:26) and Xenopus (X1FZY) (SEQ ID NO:22) sequences on the basis of their amino acid sequences. The dendrogram was constructed using the multiple alignment tool, CLUSTAL.

FIG. 2 shows a CLUSTAL multiple sequence alignment of ccs52's and FZR's. The terms FZR and ccs52 are used interchangeably herein. Zmccs52 (SEQ ID NO: 2) also referred to as ZmFZR, were aligned against the sequences AtFZR1(SEQ ID NO:14) (Genbank Accession No. NM_{—}118420), AtFZR2 (SEQ ID NO:16) (Genbank Accession No. AAM91234), AtFZR3(SEQ ID NO:18) (Genbank Accession No. NP_{—}196888), DmFZR(SEQ ID NO:20) (Genbank Accession No. CAA74575.1), HsFZR1 (SEQ ID NO:24) (Genbank Accession No. NP_{—}001129670) , MmFZR (SEQ ID NO:26) (Genbank Accession No. NP_{—}062731), X1FZR (SEQ ID NO:22) (Genbank Accession No. CAA74576), OsFZR (SEQ ID NO:30) (Genbank Accession No. NP_{—}001048804), SbFZR (SEQ ID NO:4), GmFZR1 (SEQ ID NO:6), GmFZR2 (SEQ ID NO:8), MsCcs52 (SEQ ID NO:28), GmFZR3 (SEQ ID NO:10), GmFZR4 (SEQ ID NO:12). A consensus sequence from the alignment of the sequences is shown in the bottom line (SEQ ID NO:31).

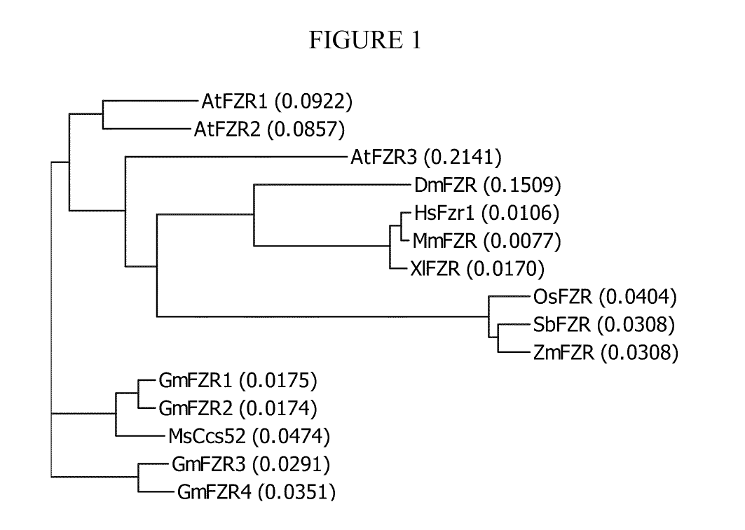

### BRIEF DESCRIPTION OF THE SEQUENCES

The application provides details of ccs52 sequences as shown in Table 1 below.

TABLE 1
Sequence Table

| SEQ ID | pnt or | | |
|---|---|---|---|
| NO: | ppt | Length | Identification |
| 1 | pnt | 1434 | Zm ccs52, cDNA |
| 2 | ppt | 477 | Zmccs52, amino acid sequence |
| 3 | pnt | 1416 | Sorghum (Sb) ccs52, cDNA |
| 4 | ppt | 471 | Sorghum (Sb) ccs52, amino acid sequence |
| 5 | pnt | 1380 | Glycine max ccs52-1, cDNA |
| 6 | ppt | 459 | Glycine max ccs52-1, amino acid sequence |
| 7 | pnt | 1398 | Glycine max ccs52-2, cDNA |
| 8 | ppt | 465 | Glycine max ccs52-2, amino acid sequence |
| 9 | pnt | 1380 | Glycine max ccs52-3, cDNA |
| 10 | ppt | 459 | Glycine max ccs52-3, amino acid sequence |
| 11 | pnt | 1359 | Glycine max ccs52-4, cDNA |
| 12 | ppt | 452 | Glycine max ccs52-4, amino acid sequence |
| 13 | pnt | 1428 | AtFZR1, Arabidopsis FZR1/ccs52 cDNA |
| | | | sequence |
| 14 | ppt | 475 | AtFZR1, Arabidopsis FZR1/ccs52 amino acid |
| | | | sequence |
| 15 | pnt | 1452 | AtFZR2, Arabidopsis FZR2/ccs52 cDNA |
| | | | sequence |
| 16 | ppt | 483 | AtFZR2, Arabidopsis FZR2/ccs52 amino acid |
| | | | sequence |
| 17 | pnt | 1446 | AtFZR3, Arabidopsis FZR3/ccs52 cDNA |
| | | | sequence |
| 18 | ppt | 481 | AtFZR3, Arabidopsis FZR32/ccs52 amino acid |
| | | | sequence |
| 19 | pnt | 1581 | DmFZR, Drosophila FZR cDNA sequence |
| 20 | ppt | 526 | DmFZR, Drosophila FZR amino acid sequence |
| 21 | pnt | 1482 | XlFZR, Xenopus FZR cDNA sequence |
| 22 | ppt | 493 | XlFZR, Xenopus FZR amino acid sequence |
| 23 | pnt | 1491 | HsFZR1, human FZR1 cDNA sequence |
| 24 | ppt | 496 | HsFZR1, human FZR1 amino acid sequence |
| 25 | pnt | 1482 | MmFZR1, mouse FZR1 cDNA sequence |
| 26 | ppt | 493 | MmFZR1, mouse FZR1 amino acid sequence |
| 27 | pnt | 1428 | MsCcs52, alfalfa Ccs52 cDNA sequence |
| 28 | ppt | 475 | MsCcs52, alfalfa Ccs52 amino acid sequence |
| 29 | pnt | 1410 | OsFZR, rice FZR cDNA sequence |
| 30 | ppt | 469 | OsFZR, rice FZR amino acid sequence |
| 31 | ppt | 525 | Consensus amino acid sequence for ccs52/FZR |

### DETAILED DESCRIPTION OF THE INVENTION

Unless defined otherwise, all technical and scientific terms used herein have the same meaning as commonly understood by one of ordinary skill in the art to which this invention belongs.

Many modifications and other embodiments of the invention set forth herein will come to mind to one skilled in the art to which this invention pertains, having the benefit of the teachings presented in the descriptions and the drawings herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The articles “a” and “an” are used herein to refer to one or more than one (i.e., to at least one) of the grammatical object of the article. By way of example, “an element” means one or more than one element.

### I. Introduction

The present invention provides novel compositions and methods for modulating, for example, increasing or decreasing, the level of cell cycle switch52 (ccs52) protein in a plant, in particular in non-germline plant cells, for example, plant cells that express photosynthetic genes. As used herein, the term ccs52 includes known ccs52 or “Fizzy-Related” (FZR) family of proteins and the novel sequences disclosed herein. In some cases, the plant cells are plant cells committed to becoming differentiated plant cells having chlorophyll, differentiated plant cells having chlorophyll or both. For example, “plant cells committed to becoming differentiated plant cells having chlorophyll” includes, but is not limited to, plant cells such as precursor cells to photosynthetic cells, e.g. dividing leaf cells, as well as differentiating mesophyll cells. “Differentiated plant cells having chlorophyll” includes, but is not limited to, cells such as photosynthetic cells, e.g. leaf cells. Differentiated refers to cells that have a specialized function or form.

Known ccs52 polynucleotides and ccs52 polypeptides or novel polynucleotides and ccs52 polypeptides of the present invention can be used to generate transgenic plants expressing ccs52's. The present inventors have discovered a novel maize ccs52 polynucleotide which encodes a polypeptide, a novel sorghum ccs52 polynucleotide which encodes a polypeptide, and four novel soybean ccs52 polynucleotides which encode polypeptides. Known polynucleotides encoding ccs52 polypeptides can be used to generate transgenic plants expressing ccs52's for use in methods of increasing plant yield, growth or both. Known ccs52's include but are not limited to a particular source and include synthetic or natural polynucleotides, those from dicots, monocots, vertebrates, or invertebrates. Exemplary ccs52's are described elsewhere herein. Modulation of the ccs52's of the present invention or known ccs52's would provide a mechanism for manipulating a plant's yield or growth. Thus, the present invention provides methods for modulating, for example, increasing or decreasing, a plant's yield and/or growth using known ccs52 polynucleotides and polypeptides or ccs52 polynucleotides and polypeptides of the present invention. In specific embodiments, methods are provided to increase yield while maintaining fertility. Expression of ccs52 in non-germline plant cells or non-meristematic plant cells, for example, expression of ccs52 in cells in which photosynthetic promoters are active, such as plant cells committed to becoming differentiated plant cells having chlorophyll, differentiated plant cells having chlorophyll or both, will not affect the plant's germline thus advantageously allowing for the production of fertile plants with increased ploidy levels, for example, in leaf cells. Expression of the ccs52's of the invention may be controlled so that it is not expressed or effectively expressed in meristematic plant cells or those involved in germline function, such as tassel or ear cells. As used herein, meristematic plant cells refers to plant cells that divide and contribute to the somatic and gametic body of the plant.

Compositions include plants having altered levels and/or activities of ccs52. ccs52 polypeptides employed in the invention share sequence identity with members of the ccs52 or “Fizzy-Related” (FZR) family of proteins. The ccs52 may be known or novel. Exemplary known ccs52's include but are not limited to those in Arabidopsis, Drosophila, Xenopus, Homo sapiens, rice, alfalfa, and mouse, and any conservatively modified variants, regardless of source, and any other variants which retain the biological properties of the ccs52, for example, ccs52 activity as disclosed herein. The sequences for these and other ccs52's can be found in NCBI's Genbank as well as in other public resources, databases or publications. See also SEQ ID NOS: 13-30.

Novel ccs52's include Zmccs52, Sorghum-ccs52, and Gm-ccs52's (SEQ ID NOS:1, 2, 3, 4, 5, 6, 7, 8, 9, 10, 11 and 12), any conservatively modified variants, regardless of source, and any other variants which retain the biological properties of the ccs52, for example, ccs52 activity as disclosed herein. Novel ccs52 genes have been identified in maize, sorghum and soybean and the cDNAs and amino acid sequences described herein. The maize ccs52 is a homolog to the Medicago sativum ccs52 gene. (See Cebolla et al., 1999: EMBO J 18(16) 4476-4484). The Zmccs52 cDNA (SEQ ID NO: 1) shares approximately 50% overall nucleic acid identity with Arabidopsis FZR1 (ccs52) (SEQ ID NO: 13) (Genbank Accession No. NM_{—}117262), approximately 50% overall nucleic acid identity with Arabidopsis FZR2 (ccs52) (SEQ ID NO: 15) (Genbank Accession No. AY128834), approximately 51% overall nucleic acid identity with Arabidopsis FZR3 (ccs52) (SEQ ID NO: 17) (Genbank Accession No. NM_{—}121387), approximately 52% overall nucleic acid identity with Medigaco sativa ccs52 (SEQ ID NO: 27) (Genbank Accession No. AF079404), approximately 51% overall nucleic acid identity with Glycine max FZR1 ccs52 (SEQ ID NO: 5), approximately 51% overall nucleic acid identity with Glycine max FZR2 ccs52 (SEQ ID NO: 7), approximately 50% overall nucleic acid identity with Glycine max FZR3 ccs52 (SEQ ID NO: 9), approximately 48% overall nucleic acid identity with Glycine max FZR4 ccs52 (SEQ ID NO: 11), approximately 85% overall nucleic acid identity with oryza sativa ccs52 (SEQ ID NO: 29) (Genbank Accession No. AP003994), and approximately 92% overall nucleic acid identity with Sorghum bicolor (Sb) ccs52 (SEQ ID NO: 3) using Align X.

The Sorghum bicolor (Sb) cDNA (SEQ ID NO: X) shares approximately 51% overall nucleic acid identity with Arabidopsis FZR1 (ccs52) (SEQ ID NO: 13) (Genbank Accession No. Accession NM_{—}117262), approximately 50% overall nucleic acid identity with Arabidopsis FZR2 (ccs52) (SEQ ID NO: 15) (Genbank Accession No. AY128834), approximately 52% overall nucleic acid identity with Arabidopsis FZR3 (ccs52) (SEQ ID NO: 17) (Genbank Accession No. NM_{—}121387), approximately 52% nucleic acid identity with Medigaco sativa ccs52 (SEQ ID NO: 27) (Genbank Accession No. AF079404), approximately 52% overall nucleic acid identity with Glycine max FZR1 ccs52 (SEQ ID NO: 5), approximately 53% overall nucleic acid identity with Glycine max FZR2 ccs52 (SEQ ID NO: 7), approximately 50% overall nucleic acid identity with Glycine max FZR3 ccs52 (SEQ ID NO: 9), approximately 50% overall nucleic acid identity with Glycine max FZR4 ccs52 (SEQ ID NO: 11), approximately 87% nucleic acid identity with oryza sativa ccs52 (SEQ ID NO: 29) (Genbank Accession No. AP003994), and approximately 92% nucleic acid identity with Zmccs52 ccs52 (SEQ ID NO: 1) using Align X.

The GmFZR1 cDNA (SEQ ID NO: 5) shares approximately 53% overall nucleic acid identity with oryza sativa ccs52 (SEQ ID NO: 29) (Genbank Accession No. AP003994), approximately 51% nucleic acid identity with Zmccs52 ccs52 (SEQ ID NO: 1), and approximately 51% nucleic acid identity with Sorghum bicolor (Sb) ccs52 (SEQ ID NO: 3) using Align X.

The GmFZR2 cDNA (SEQ ID NO: 7) shares approximately 52% overall nucleic acid identity with oryza sativa ccs52 (SEQ ID NO: 29) (Genbank Accession No. AP003994), approximately 51% nucleic acid identity with Zmccs52 ccs52 (SEQ ID NO: 1), and approximately 53% nucleic acid identity with Sorghum bicolor (Sb) ccs52 (SEQ ID NO: 3) using Align X.

The GmFZR3 cDNA (SEQ ID NO: 9) shares approximately 51% overall nucleic acid identity with oryza sativa ccs52 (SEQ ID NO: 29) (Genbank Accession No. AP003994), approximately 50% nucleic acid identity with Zmccs52 ccs52 (SEQ ID NO: 1), and approximately 51% nucleic acid identity with Sorghum bicolor (Sb) ccs52 (SEQ ID NO: 3) using Align X.

The GmFZR43 cDNA (SEQ ID NO: 11) shares approximately 51% overall nucleic acid identity with oryza sativa ccs52 (SEQ ID NO: 29) (Genbank Accession No. AP003994), approximately 48% nucleic acid identity with Zmccs52 ccs52 (SEQ ID NO: 1), and approximately 50% nucleic acid identity with Sorghum bicolor (Sb) ccs52 (SEQ ID NO: 3) using Align X.

The Zmccs52 polypeptide (SEQ ID NO: 2) shares approximately 39% overall amino acid identity with Arabidopsis FZR1 (ccs52) (SEQ ID NO: 14) (Genbank Accession No. NM_{—}118420), approximately 38% overall amino acid identity with Arabidopsis FZR2 (ccs52) (SEQ ID NO: 16) (Genbank Accession No. AAM91234), approximately 38% overall amino acid identity with Arabidopsis FZR3 (ccs52) (SEQ ID NO: 18) (Genbank Accession No. NP_{—}196888), approximately 37% overall amino acid identity with Medigaco sativa ccs52 (SEQ ID NO: 28) (Genbank Accession No. AAD22612), approximately 39% overall amino acid identity with Glycine max FZR1 ccs52 (SEQ ID NO: 6), approximately 38% overall amino acid identity with Glycine max FZR2 ccs52 (SEQ ID NO: 8), approximately 39% overall amino acid identity with Glycine max FZR3 ccs52 (SEQ ID NO: 10), approximately 39% overall amino acid identity with Glycine max FZR4 ccs52 (SEQ ID NO: 12), approximately 89% overall amino acid identity with oryza sativa ccs52 (SEQ ID NO: 30) (Genbank Accession No. NP_{—}001048804), and approximately 93% overall amino acid identity with Sorghum bicolor (Sb) ccs52 (SEQ ID NO: 4) using Align X.

The Sorghum bicolor (Sb) polypeptide (SEQ ID NO: 4) shares approximately 38% overall amino acid identity with Arabidopsis FZR1 (ccs52) (SEQ ID NO: 14) (Genbank Accession No. NM_{—}118420), approximately 37% overall amino acid identity with Arabidopsis FZR2 (ccs52) (SEQ ID NO: 16) (Genbank Accession No. AAM91234), approximately 39% overall amino acid identity with Arabidopsis FZR3 (ccs52) (SEQ ID NO: 18) (Genbank Accession No. NP_{—}196888), approximately 38% amino acid identity with Medigaco sativa ccs52 (SEQ ID NO: 28) (Genbank Accession No. AAD22612), approximately 39% overall amino acid identity with Glycine max FZR1 ccs52 (SEQ ID NO: 6), approximately 38% overall amino acid identity with Glycine max FZR2 ccs52 (SEQ ID NO: 8), approximately 40% overall amino acid identity with Glycine max FZR3 ccs52 (SEQ ID NO: 10), approximately 40% overall amino acid identity with Glycine max FZR4 ccs52 (SEQ ID NO: 12), approximately 93% amino acid identity with oryza sativa ccs52 (SEQ ID NO: 30) (Genbank Accession No. NP_{—}001048804), and approximately 93% amino acid identity with Zmccs52 ccs52 (SEQ ID NO: 2) using Align X.

The GmFZR1 polypeptide (SEQ ID NO: 6) shares approximately 38% overall amino acid identity with oryza sativa ccs52 (SEQ ID NO: 30) (Genbank Accession No. NP_{—}001048804), approximately 39% amino acid identity with Zmccs52 ccs52 (SEQ ID NO: 2), and approximately 39% amino acid identity with Sorghum bicolor (Sb) ccs52 (SEQ ID NO: 4) using Align X.

The GmFZR2 polypeptide (SEQ ID NO: 8) shares approximately 38% overall amino acid identity with oryza sativa ccs52 (SEQ ID NO: 30) (Genbank Accession No. NP_{—}001048804), approximately 38% amino acid identity with Zmccs52 ccs52 (SEQ ID NO: 2), and approximately 38% amino acid identity with Sorghum bicolor (Sb) ccs52 (SEQ ID NO: 4) using Align X.

The GmFZR3 polypeptide (SEQ ID NO: 10) shares approximately 40% overall amino acid identity with oryza sativa ccs52 (SEQ ID NO: 30) (Genbank Accession No. NP_{—}001048804), approximately 39% amino acid identity with Zmccs52 ccs52 (SEQ ID NO: 2), and approximately 40% amino acid identity with Sorghum bicolor (Sb) ccs52 (SEQ ID NO: 4) using Align X.

The GmFZR4 polypeptide (SEQ ID NO: 12) shares approximately 40% overall amino acid identity with oryza sativa ccs52 (SEQ ID NO: 30) (Genbank Accession No. NP_{—}001048804), approximately 39% amino acid identity with Zmccs52 ccs52 (SEQ ID NO: 2), and approximately 40% amino acid identity with Sorghum bicolor (Sb) ccs52 (SEQ ID NO: 4) using Align X.

In specific compositions, the plants have an altered level and/or activity of a known or novel ccs52 polypeptide. Any plant FZR/ccs52 may be used. The plant or plant cell or plant part may comprise one or more additional copies of a nucleic acid that occurs naturally in the same plant species or variety, a nucleic acid that is from a different species, variety or plant, or one that does not occur in nature.

In some examples, the plants have an altered level and/or activity of a ccs52 polypeptide having the amino acid sequence set forth in SEQ ID NO:2, 4, 6, 8, 10, 12, 14, 16, 18, 20, 22, 24, 26, 28, or 30. Further provided are plants having an altered level and/or activity of the ccs52 polypeptide encoded by a polynucleotide set forth in SEQ ID NO:1, 3, 5, 7, 9, 11, 13, 15, 17, 19, 21, 23, 25, 27, or 29 or an active variant or fragment thereof. In some examples, the plants have an altered level and/or activity of a ccs52 polypeptide having the amino acid sequence set forth in SEQ ID NO: 2, 4, 6, 8, 10 or 12 or an active variant or fragment thereof. Further provided are plants having an altered level and/or activity of the ccs52 polypeptide encoded by a polynucleotide set forth in SEQ ID NO: 1, 3, 5, 7, 9, or 11 or an active variant or fragment thereof. The plants of the invention may exhibit modulation in yield and/or growth. For example, the plants may have modulated ear size, seed set, total number of seeds, seed size, seed volume, number of filled seeds, total seed weight per plant, chlorophyll levels in plant cells having chlorophyll, photosynthetic machinery, cell size of plant cells committed to becoming differentiated plant cells having chlorophyll, cell size of differentiated plant cells having chlorophyll, overall source levels of photosynthate, harvest index, thousand kernel weight, number of tillers, number of first panicles (being the tallest panicle and all the panicles that overlap with the tallest panicle when aligned vertically), number of second panicles, and plant biomass as compared to an appropriate control plant cell or plant. In some cases, the plants transgenic for a ccs52 of the present invention will have increased ear size, increased seed set, increased chlorophyll levels in plant cells having chlorophyll, increased levels or amount of photosynthetic machinery, increased cell size of plant cells committed to becoming differentiated plant cells having chlorophyll, increased cell size of differentiated plant cells having chlorophyll, or increased overall source levels of photosynthate as compared to an appropriate control plant cell or plant.

In specific embodiments, the plants have stably incorporated into their genomes a ccs52 sequence. Suitable promoters for the use in expression a ccs52 sequence is described elsewhere herein. By “phenotypic change” is intended a measurable change in one or more cell functions. For example, plants having a heterologous ccs52 polypeptide may show specific expression or activity of the ccs52 polypeptide in plant cells committed to becoming differentiated plant cells having chlorophyll, e.g. dividing leaf cells, as well as differentiating mesophyll cells, in differentiated plant cells having chlorophyll, for example, in a leaf, or both. Certain phenotypic changes may be observed at the cellular, tissue or whole-plant level, for example, a change in ear size, seed set, total number of seeds, seed size and/or volume, number of filled seeds, total seed weight per plant, chlorophyll levels in plant cells having chlorophyll, cell size of plant cells committed to becoming differentiated plant cells having chlorophyll, cell size of differentiated plant cells having chlorophyll, net photosynthesis in plants and/or plant cells having chlorophyll, overall source levels of photosynthate (i.e. total sucrose produced in the leaves to be used as an energy source to fuel ear development), harvest index, thousand kernel weight, number of tillers, number of first panicles (being the tallest panicle and all the panicles that overlap with the tallest panicle when aligned vertically), number of second panicles, and plant biomass as compared to an appropriate control plant cell, plant tissue or plant.

Various methods of genetic modification are described in more detail elsewhere herein, as are examples of phenotypes that can result from modification affecting the spatial and temporal expression, expression level and/or activity of a ccs52 sequence of the invention. In one aspect, the ccs52 sequence is expressed in plant cells committed to becoming differentiated plant cells having chlorophyll, e.g. dividing leaf cells or differentiating mesophyll cells. In one aspect, the ccs52 sequence is expressed in differentiated plant cells having chlorophyll, e.g. leaf cells. In one aspect, the ccs52 sequence is expressed in non-meristematic cells or non-germline cells. In another aspect, the ccs52 sequence is not expressed or effectively expressed in roots and/or reproductive tissues such as the tassel and ear, leaving these tissues diploid. Accordingly, plants transgenic for ccs52 may have expression in plant cells that expresses photosynthetic genes such as plant cells committed to becoming differentiated plant cells having chlorophyll and/or differentiated plant cells having chlorophyll, without expression in root cells or those cells of the reproductive tissues so that fertility of the transgenic plant or progeny is maintained.

Also described herein are methods of improving or increasing yield or growth of a plant by producing a plant transgenic for a ccs52 of the present invention. Also described herein are methods of improving or increasing yield or growth of a plant by producing a plant transgenic for a known ccs52. For example, increased yield includes without limitation increased ear size, seed set, total number of seeds, seed size and/or volume, number of filled seeds, total seed weight per plant, chlorophyll levels in plant cells having chlorophyll, photosynthetic machinery, cell size of plant cells committed to becoming differentiated plant cells having chlorophyll, cell size of differentiated plant cells having chlorophyll, cell size of differentiated plant cells having chlorophyll, the overall source levels of photosynthate, harvest index, thousand kernel weight, number of tillers, number of first panicles (being the tallest panicle and all the panicles that overlap with the tallest panicle when aligned vertically), number of second panicles of transgenic plants transgenic for ccs52 and non-transgenic control plants. Increased yield also includes an increase in biomass in one or more parts of a plant relative to the corresponding part(s) of wild-type plants. In some examples, the level of yield in a transgenic plant of the invention is at least 5%, 10%, or 20%, 30% , 40%, 50%, 60%, 70%, 80%, 90% or 100% greater than the yield exhibited in a non-transgenic control plant. The level of yield is measured by any suitable methods or techniques known to one skilled in the art.

A change in seed yield may be determined by evaluating any suitable characteristics indicative of seed yield. This includes but is not limited to a change in the biomass of the seed (seed weight), in the number of (filled) seeds, in the size of the seeds, in seed volume, as relative to corresponding control plant or plant part, e.g. a wild-type plant or plant part. Depending on the crop, the plant parts in question may be above-ground biomass (e.g. corn, when used as silage, sugarcane), roots (e.g. sugar beet), fruit (e.g. tomato), cotton fibers, or any other part of the plant. A change in seed size and/or volume may also influence the composition of seeds. A change in seed yield could be due to an change in the number and/or size of flowers. A change in yield might also change the harvest index, which is expressed as a ratio of the yield of harvestable parts, such as seeds, over the total biomass; or thousand kernel weight. A change in yield also encompasses the capacity for planting at higher or lower density (number of plants per hectare or acre).

Thus, plant are transgenic for a ccs52 of the present invention or known ccs52 in plant cells committed to becoming differentiated plant cells having chlorophyll, differentiated plant cells having chlorophyll, or both may have an increase in the biomass of the seed (seed weight), an increase in the number of (filled) seeds, an increase in the size of the seeds, an increase in seed volume, as relative to corresponding control plant or plant part, e.g. a wild-type plant or plant part. When maize is transgenic for ccs52, the increase of seed yield may be reflected, for example, as an increase of rows (of seeds) per ear and/or an increased number of kernels per row. An increase in seed size and/or volume may also influence the composition of seeds. An increase in seed yield could be due to an increase in the number and/or size of flowers. An increase in yield might also increase the harvest index, which is expressed as a ratio of the yield of harvestable parts, such as seeds, over the total biomass, or thousand kernel weight. Increased yield also encompasses the capacity for planting at higher density (number of plants per hectare or acre). When rice is transgenic for ccs52, a yield increase may be manifested by an increase in one or more of the following: number of panicles per plant, number of spikelets per panicle, number of flowers per panicle, increase in the seed filling rate, increase in thousand kernel weight, and the like.

It is also contemplated that modified cell division may contribute to yield increase. The term “modified cell division” encompasses an increase or decrease in cell division or an abnormal cell division/cytokinesis, altered plane of division, altered cell polarity, altered cell differentiation. The term also comprises phenomena such as endomitosis, acytokinesis, polyploidy, polyteny and endoreduplication. Ploidy increases in one part of the plant from expression of ccs52 in plant cells committed to becoming differentiated plant cells having chlorophyll and/or differentiated plant cells having chlorophyll, such as the leaf cells, may impact development in another part, such as the ear or seed. In this way, larger ears with higher seed sets may be produced resulting in increased yield.

Plants transgenic for a ccs52 of the present invention or known ccs52 may exhibit a modified growth rate is compared to corresponding control plants when the ccs52 is expressed as described herein. The term “modified growth rate” as used herein encompasses, but is not limited to, a faster rate of growth in one or more parts of a plant (including seeds), at one or more stages in the life cycle of a plant. Plants with improved growth may show a modified growth curve and may have modified values for their T_{mid} or T₉₀ (respectively the time needed to reach half of their maximal size or 90% of their maximal size, each relative to corresponding wild-type plants). The term “improved growth” encompasses enhanced vigor, earlier flowering, modified cycling time or combinations thereof.

Performance of the methods according to the present invention may result in plants having increased yield, in particular plants having increased seed yield. Accordingly, provided herein is a method for increasing the yield of plants. The method includes modulating expression and/or ccs52 activity of the known ccs52 or ccs52 sequence of the present invention in a plant, in particular, in plant cells committed to becoming differentiated plant cells having chlorophyll, differentiated plant cells having chlorophyll or both. In a particular embodiment, a method of increasing yield includes increasing expression and/or ccs52 activity of a ccs52 sequence of the present invention in a plant, in particular in plant cells committed to becoming differentiated plant cells having chlorophyll or in differentiated plant cells having chlorophyll or combinations thereof. When the plant is maize, the increased yield may be manifested as increased seed yield or ear size or both.

Modified plants are of interest, as are modified plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grain and the like. As used herein, “grain” means the mature seed produced by commercial growers for purposes other than advancing or reproducing the species, e.g. for such end uses as feed, food, or fiber. Progeny, variants, and mutants of the regenerated plants are also included within the scope of the invention, provided that such plants or plant parts comprise the genetic modification.

### II. Fragments and Variants

Fragments and variants of the ccs52 polynucleotides and proteins encoded thereby can be employed in the methods and compositions of the present invention. By “fragment” is intended a portion of the polynucleotide or a portion of the amino acid sequence and hence of the protein encoded thereby. Fragments of a polynucleotide may encode protein fragments that retain the biological activity of the native protein and hence retain ccs52 activity, for example, a constitutively active ccs52 created by deletion of its putative regulatory domains.

As used interchangeably herein, a “ccs52 activity”, “biological activity of ccs52” or “functional activity of ccs52”, refers to an activity exerted by a ccs52 protein, polypeptide or portion thereof as determined in vivo, ex vivo, or in vitro, according to standard techniques.

In one aspect, a ccs52 activity is at least one or more of the following activities: a modulation in ear size, a modulation in seed set, a modulation in total number of seeds, a modulation in seed size, a modulation in volume, a modulation in number of filled seeds, a modulation in total seed weight per plant, a modulation in chlorophyll levels in plant cells having chlorophyll, a modulation in levels of photosynthetic machinery, a modulation in cell size of plant cells committed to becoming differentiated plant cells having chlorophyll, a modulation in cell size of differentiated plant cells having chlorophyll, a modulation in overall source levels of photosynthate, a modulation in harvest index, a modulation in thousand kernel weight, a modulation in number of tillers, a modulation in number of first panicles, or a modulation in number of second panicles as compared to an appropriate control plant cell, plant tissue or plant; a modulation of biomass in one or more parts of a plant relative to the corresponding part(s) of wild-type plants; a modulation in the biomass of the seed (seed weight), a modulation in the number of (filled) seeds, a modulation in the size of the seeds, a modulation in seed volume, a modulation in the number of flowers, or a modulation in size of flowers; a modulation in yield of a plant; (iv) a modulation in thousand kernel weight; a modulation in the capacity for planting at higher or lower density; a modulation in the harvest index; a modulation in the number of panicles per plant, a modulation in the number of spikelets per panicle, a modulation in the number of flowers per panicle, a modulation in the seed filling rate; a modulation in cell division, a modulation in an abnormal cell division/cytokinesis, a modulation in altered plane of division, a modulation altered cell polarity, a modulation altered cell differentiation, a modulation in endomitosis, a modulation in acytokinesis, a modulation in polyploidy, or a modulation in endoreduplication; or a modified growth rate of the plant or plant cell.

In one aspect, a ccs52 activity is at least one or more of the following activities: an increase in ear size, an increase in seed set, an increase in total number of seeds, an increase in seed size, an increase in volume, an increase in number of filled seeds, an increase in total seed weight per plant, an increase in chlorophyll levels in plant cells having chlorophyll, an increase in levels of photosynthetic machinery, an increase in cell size of plant cells committed to becoming differentiated plant cells having chlorophyll, an increase in cell size of differentiated plant cells having chlorophyll, an increase in overall source levels of photosynthate, an increase in harvest index, an increase in thousand kernel weight, an increase in number of tillers, an increase in number of first panicles, or an increase in number of second panicles as compared to an appropriate control plant cell, plant tissue or plant; an increase of biomass in one or more parts of a plant relative to the corresponding part(s) of wild-type plants; an increase in the biomass of the seed (seed weight), an increase in the number of (filled) seeds, an increase in the size of the seeds, an increase in seed volume, an increase in the number of flowers, or an increase in size of flowers; an increase in yield of a plant; an increase in thousand kernel weight; an increase in the capacity for planting at higher or lower density; an increase in the harvest index; an increase in the number of panicles per plant, an increase in the number of spikelets per panicle, an increase in the number of flowers per panicle, an increase in the seed filling rate; an increase in cell division, an increase in an abnormal cell division/cytokinesis, an increase in altered plane of division, an increase altered cell polarity, an increase altered cell differentiation, an increase endomitosis, an increase in acytokinesis, an increase in polyploidy, or an increase in endoreduplication; or a modified growth rate of the plant or plant cell.

Alternatively, fragments of a polynucleotide that are useful as hybridization probes or PCR primers generally do not encode fragment proteins retaining biological activity. Thus, fragments of a nucleotide sequence may range from at least about 20 nucleotides, about 50 nucleotides, about 100 nucleotides, up to the full-length polynucleotide encoding the proteins employed in the invention.

A fragment of a ccs52 polynucleotide that encodes a biologically active portion of a ccs52 protein employed in the invention will encode at least 15, 25, 50, 75, 100, 125, 150, 175, 200, 200, 250, 300, 350, 400, 450 or 500 contiguous amino acids, or up to the total number of amino acids present in a full-length or partial ccs52 protein of the invention (for example, 529 amino acids for SEQ ID NO: 2).

A biologically active portion of a ccs52 protein can be prepared by isolating a portion of one of the ccs52 polynucleotides employed in the invention, expressing the encoded portion of the ccs52 protein (e.g., by recombinant expression in vitro), and assessing the activity of the encoded portion of the ccs52 protein. Polynucleotides that are fragments of a ccs52 nucleotide sequence comprise at least 15, 20, 50, 75, 100, 150, 200, 250, 300, 350, 500, 550, 500, 550, 600, 650, 700, 800, 900, 1,000, 1,100, 1200, 1300, or 1400 nucleotides, or up to the number of nucleotides present in a full-length ccs52 polynucleotide disclosed herein (for example, 1587 nucleotides for SEQ ID NO:1).

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the ccs52 polypeptides. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a ccs52 protein employed in the invention. Generally, variants of a particular polynucleotide of the invention will have at least about 50%, 55%, 50%, 55%, 60%, 65%, 70%, 75%, 80%, 85%, 90%, 91%, 92%, 93%, 95%, 95%, 96%, 97%, 98%, 99% or more sequence identity to that particular polynucleotide as determined by sequence alignment programs and parameters described elsewhere herein.

Variants of a particular polynucleotide employed in the invention (i.e., the reference polynucleotide) can also be evaluated by comparison of the sequence identity between the polypeptide encoded by a variant polynucleotide and the polypeptide encoded by the reference polynucleotide. Thus, for example, an isolated polynucleotide that encodes a polypeptide with a given percent sequence identity to a polypeptide of SEQ ID NO: 2, 4, 6, 8, 10 or 12 is encompassed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 50%, 55%, 50%, 55%, 60%, 65%, 70%, 75%, 80%, 85%, 90%, 91%, 92%, 93%, 95%, 95%, 96%, 97%, 98%, 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, ccs52 activity as described herein. Such variants may result from, for example, genetic polymorphism or from human manipulation. Biologically active variants of a native ccs52 protein of the invention will have at least about 50%, 55%, 60%, 65%, 70%, 75%, 80%, 85%, 90%, 91%, 92%, 93%, 95%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 5, 3, 2, or even 1 amino acid residue.

The proteins employed in the methods of the invention may be altered in various ways including amino acid substitutions, deletions, truncations, and insertions. Methods for such manipulations are generally known in the art. For example, amino acid sequence variants and fragments of the ccs52 proteins can be prepared by mutations in the DNA. Methods for mutagenesis and polynucleotide alterations are well known in the art. See, for example, Kunkel (1985) Proc. Natl. Acad. Sci. USA 82:588-592; Kunkel et al. (1987) Methods in Enzymol. 155:367-382; U.S. Pat. No. 5,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal. Variants of ccs52 polypeptides can also include isolating natural variants from plants cells that exist in nature or creating recombinant ccs52's.

Thus, the genes and polynucleotides employed in the invention include both the naturally-occurring sequences as well as mutant forms. Likewise, the proteins employed in the invention encompass naturally occurring proteins as well as variations and modified forms thereof. Such variants will continue to possess the desired ccs52 activity. Obviously, the mutations that will be made in the DNA encoding the variant must not place the sequence out of reading frame and optimally will not create complementary regions that could produce secondary mRNA structure.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity and/or expression can be evaluated by in gel kinase assays, real time RT-PCR analysis, Northern, Westerns, electrophoretic mobility shift assays, DNAse I footprinting assays and the like. (Shou et al. Expression of the Nicotiana protein kinase (NPK1) enhanced drought tolerance in transgenic maize. (2004). J Exp Bot. 55(399): 1013-9). Assays for detecting such activity or expression are known to one skilled in the art. Alternately, they are described in detail elsewhere herein. For example, an oligonucleotide of at least 15, 30, 50, 100, 200, 300, 400, 500, 600, 700, 800, 900 or 1000 nucleotides in length and sufficient to specifically hybridize under stringent conditions to ccs52 mRNA may be used in Northern blot analysis. Ccs52 proteins may be detected using a labeled antibody capable of binding to known ccs52 proteins or ccs52 proteins of the present invention. Antibodies can be polyclonal, or more preferably, monoclonal. An isolated ccs52 protein, or fragment thereof, can be used as an immunogen to generate antibodies that bind specifically to known ccs52's or ccs52's of the present invention using standard techniques for polyclonal and monoclonal antibody preparation. Techniques for detection of ccs52 protein include enzyme linked immunosorbent assays (ELISAs), Western blots, immunoprecipitations and immunofluorescence.

Variant polynucleotides and proteins also encompass sequences and proteins derived from a mutagenic and recombinogenic procedure such as DNA shuffling. With such a procedure, one or more different ccs52 coding sequences can be manipulated to create a new ccs52 possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the ccs52 cDNA or gene of the invention and other known ccs52 cDNA or genes to obtain a new gene coding for a protein with an improved property of interest, such as an increased Kₘ in the case of an enzyme. Strategies for such DNA shuffling are known in the art. See, for example, Stemmer (1995) Proc. Natl. Acad. Sci. USA 91:10757-10751; Stemmer (1995) Nature 370:389-391; Crameri et al. (1997) Nature Biotech. 15:536-538; Moore et al. (1997) J. Mol. Biol. 272:336-357; Zhang et al. (1997) Proc. Natl. Acad. Sci. USA 95:5505-5509; Crameri et al. (1998) Nature 391:288-291; and U.S. Pat. Nos. 5,605,793 and 5,837,558.

The polynucleotides employed in the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire ccs52 sequence set forth in SEQ ID NO: 1, 3, 5, 7, 9, or 11 or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 95%, 95%, 96%, 97%, 98%, 99%, or greater sequence identity. Functions of orthologs are often highly conserved among species. Thus, isolated polynucleotides that encode a ccs52 protein and which hybridize under stringent conditions to the sequence of SEQ ID NO: 1, 3, 5, 7, 9, or 11 or to complements, variants, or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or another detectable marker. Thus, for example, probes for hybridization can be made by labeling synthetic oligonucleotides based on the ccs52 polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire ccs52 polynucleotide such as those known or disclosed herein, or one or more portions thereof, may be used as a probe capable of specifically hybridizing to corresponding ccs52 polynucleotide and messenger RNAs. To achieve specific hybridization under a variety of conditions, such probes include sequences that are unique among ccs52 polynucleotide sequences and are optimally at least about 10 nucleotides in length, and most optimally at least about 20 nucleotides in length. Such probes may be used to amplify corresponding ccs52 polynucleotide from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 50 to 55% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 25 hours, usually about 5 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1985) Anal. Biochem. 138:267-285: Tₘ=81.5° C.+16.6 (log M)+0.51 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 5° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 15, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 55° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 50, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 5:11-17; the local alignment algorithm of Smith et al. (1981) Adv. Appl. Math. 2:582; the global alignment algorithm of Needleman and Wunsch (1970) J. Mol. Biol. 58:553-553; the search-for-local alignment method of Pearson and Lipman (1988) Proc. Natl. Acad. Sci. 85:2555-2558; the algorithm of Karlin and Altschul (1990) Proc. Natl. Acad. Sci. USA 872265, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA Accelrys GCG (Accelrys Inc., 9685 Scranton Road, San Diego, Calif., USA). Alignments using these programs can be performed using the default parameters. The CLUSTAL program is well described by Higgins et al. (1988) Gene 73:237-255 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1995) Meth. Mol. Biol. 25:307-331. The ALIGN program is based on the algorithm of Myers and Miller (1988) supra. A PAM120 weight residue table, a gap length penalty of 12, and a gap penalty of 5 can be used with the ALIGN program when comparing amino acid sequences. The BLAST programs of Altschul et al (1990) J. Mol. Biol. 215:503 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. The United States' National Center for Biotechnology Information and the European Bioinformatics Institute of the European Molecular Biology Laboratory provide such tools, as do various commercial entities known to those of skill in the art. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix.

GAP uses the algorithm of Needleman and Wunsch (1970) J. Mol. Biol. 58:553-553, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 5, 5, 6, 7, 8, 9, 10, 15, 20, 25, 30, 35, 50, 55, 50, 55, 60, 65 or greater.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 5 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

### III. Providing Sequences

The ccs52 sequences, known or novel, can be introduced/expressed in a host cell such as bacteria, yeast, insect, mammalian, or optimally plant cells. It is expected that those of skill in the art are knowledgeable in the numerous systems available for the introduction of a polypeptide or a nucleotide sequence of the present invention into a host cell. No attempt to describe in detail the various methods known for providing proteins in prokaryotes or eukaryotes will be made.

By “host cell” is meant a cell which comprises a heterologous nucleic acid sequence of the invention. Host cells may be prokaryotic cells such as E. coli, or eukaryotic cells such as yeast, insect, amphibian, or mammalian cells. Host cells can also be monocotyledonous or dicotyledonous plant cells. In one embodiment, the monocotyledonous host cell is a maize host cell.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally-occurring molecules and synthetic analogues. The polynucleotides of the invention also encompass all forms of sequences including, but not limited to, single-stranded forms, double-stranded forms, hairpins, stem-and-loop structures, and the like.

A ccs52 polynucleotide employed of the invention can be provided in expression cassettes for expression in the plant of interest. The cassette will include 5′ and 3′ regulatory sequences operably linked to a ccs52 polynucleotide. “Operably linked” is intended to mean a functional linkage between two or more elements. For example, an operable linkage between a polynucleotide of interest and a promoter is a functional link that allows for expression of the polynucleotide of interest. Operably linked elements may be contiguous or non-contiguous. When used to refer to the joining of two protein coding regions, operably linked means that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the ccs52 polynucleotide to be under the transcriptional regulation of the regulatory regions. The expression cassette may additionally contain selectable marker genes.

The expression cassette will include, in the 5′-3′ direction of transcription, a transcriptional and translational initiation region (i.e., a promoter), a ccs52 polynucleotide, known or of the invention, and a transcriptional and translational termination region (i.e., termination region) functional in plants. The regulatory regions (including promoters, transcriptional regulatory regions, and translational termination regions) and/or the employed ccs52 polynucleotide may be native/analogous to the host cell and/or to each other. Alternatively, the regulatory regions and/or the employed ccs52 polynucleotide may be foreign/heterologous to the host cell and/or to each other. As used herein, “heterologous” in reference to a sequence is a sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are modified from their original form and/or genomic locus, or the promoter is not the native promoter for the operably linked polynucleotide. As used herein, a chimeric gene comprises a coding sequence operably linked to a promoter that is heterologous to the coding sequence. While it may be optimal to express the sequences using heterologous promoters, the native promoter sequences may be used. Such constructs can change the expression levels of the ccs52 in the plant or plant cell. Thus, the phenotype of the plant or plant cell can be altered.

The termination region may be native with the transcriptional initiation region, may be native with the operably linked ccs52 polynucleotide of interest, may be native with the plant host, or may be derived from another source (i.e., foreign or heterologous) to the promoter, the ccs52 polynucleotide of interest, the plant host, or any combination thereof. Convenient termination regions are available from the Ti— plasmid of A. tumefaciens, such as the octopine synthase and nopaline synthase termination regions. See also Guerineau et al. (1991) Mol. Gen. Genet. 262:151-155; Proudfoot (1991) Cell 65:671-675; Sanfacon et al. (1991) Genes Dev. 5:151-159; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant by using plant-preferred codons. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,536,391, and Murray et al. (1989) Nucleic Acids Res. 17:577-598, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 155:9-20), and human immunoglobulin heavy-chain binding protein (BiP) (Macejak et al. (1991) Nature 353:90-95); untranslated leader from the coat protein mRNA of alfalfa mosaic virus (AMV RNA 5) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 85:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,5-dichlorophenoxyacetate (2,5-D). Additional selectable markers include phenotypic markers such as β-galactosidase and fluorescent proteins such as green fluorescent protein (GFP) (Su et al. (2005) Biotechnol Bioeng 85:610-9 and Fetter et al. (2005) Plant Cell 16:215-28), cyan florescent protein (CYP) (Bolte et al. (2005) J. Cell Science 117:953-55 and Kato et al. (2002) Plant Physiol 129:913-52), and yellow florescent protein (PhiYFP™ from Evrogen, see, Bolte et al. (2005) J. Cell Science 117:953-55). For additional selectable markers, see generally, Yarranton (1992) Curr. Opin. Biotech. 3:506-511; Christopherson et al. (1992) Proc. Natl. Acad. Sci. USA 89:6315-6318; Yao et al. (1992) Cell 71:63-72; Reznikoff (1992) Mol. Microbiol. 6:2519-2522; Barkley et al. (1980) in The Operon, pp. 177-220; Hu et al. (1987) Cell 58:555-566; Brown et al. (1987) Cell 59:603-612; Figge et al. (1988) Cell 52:713-722; Deuschle et al. (1989) Proc. Natl. Acad. Sci. USA 86:5500-5505; Fuerst et al. (1989) Proc. Natl. Acad. Sci. USA 86:2559-2553; Deuschle et al. (1990) Science 258:580-583; Gossen (1993) Ph. D. Thesis, University of Heidelberg; Reines et al. (1993) Proc. Natl. Acad. Sci. USA 90:1917-1921; Labow et al. (1990) Mol. Cell. Biol. 10:3353-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:5657-5653; Hillenand-Wissman (1989) Topics Mol. Struc. Biol. 10:153-162; Degenkolb et al. (1991) Antimicrob. Agents Chemother. 35:1591-1595; Kleinschnidt et al. (1988) Biochemistry 27:1095-1105; Bonin (1993) Ph. D. Thesis, University of Heidelberg; Gossen et al. (1992) Proc. Natl. Acad. Sci. USA 89:5557-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 335:721-725. Such disclosures are herein incorporated by reference. The above list of selectable marker genes is not meant to be limiting. Any selectable marker gene can be used in the present invention.

A number of promoters can be used in the practice of the invention. “Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers.

The promoters can be selected based on the desired outcome. The nucleic acids can be combined with tissue-preferred, constitutive, inducible, such as chemically-inducible promoters, or other promoters for expression in plants. Preferably, the promoters used to express the ccs52's sequences do not express or effectively express in meristematic or reproductive tissues so that fertility of the plant transgenic for the ccs52 is maintained and not compromised.

Tissue-preferred promoters can be utilized to target enhanced expression of ccs52 (FZR) within a particular plant tissue. By “tissue-preferred” is intended to mean that expression is predominantly in a particular tissue, albeit not necessarily exclusively in that tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 255(3):337-353; Russell et al. (1997) Transgenic Res. 6(2):157-168; Rinehart et al. (1996) Plant Physiol. 112(3):1331-1351; Van Camp et al. (1996) Plant Physiol. 112(2):525-535; Canevascini et al. (1996) Plant Physiol. 112(2):513-525; Yamamoto et al. (1995) Plant Cell Physiol. 35(5):773-778; Lam (1995) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 5(3):595-505. Such promoters can be modified, if necessary, for weak expression. See, also, U.S. Patent Application No. 2003/0074698, herein incorporated by reference. Other suitable promoters include endosperm promoters, such as early endosperm promoters including but not limited to ZM-LEG1 (Abbitt & Jung. 2007. U.S. Pat. No. 7,211,712 B2), Gamma Zein (Uead et al., 1994. Mol. Cell boil. 14:4350-4359), Gamma-kafarin promoter (Mishra et al., 2008. Mol Biol Rep. 35:81-88), Glb1 promtoer (Liu et al. 1998. Plant Cell Reports 17:650-655, and EEP1 (Habben et al. US20070169226).

Any promoter that directs gene expression in plant photosynthetic cells or tissue may be used in the compositions, vectors, constructs, and cassettes, and methods described herein. In one aspect, the promoter preferentially or specifically directs gene expression in plant photosynthetic cells or tissue. In some examples a polynucleotide of interest such as a ccs52 polynucleotide is expressed in plant cells that express photosynthetic genes. Any promoter that preferentially or specifically directs leaf-specific or leaf-preferred gene expression may be used in the compositions, vectors, constructs, and cassettes, and methods described herein. Preferably the promoter preferentially expresses the polynucleotide in plant cells committed to becoming differentiated plant cells having chlorophyll, differentiated plant cells having chlorophyll or both may be used to express the ccs52 polynucleotide. For example, the RUBISCO SSU promoter may be used. (Mazur & Chua, 1985. NAR 13: 2373-2386). Any promoter driving expression in dividing leaf cells or differentiating mesophyll cells may be used to express the ccs52 polynucleotide. Any promoter driving leaf-specific or leaf-preferred nuclear-encoded gene expression may be used. In some cases, the promoters are light-inducible or promoters for genes whose encoded products are chloroplast-targeted. Suitable leaf-preferred or leaf-specific promoters are known in the art. These include but are not limited to Cab-M1 and rbcS-m3 promoters (Bansal et al., 1992. PNAS 89:3654-3658), Lhca3.St.1 promoter from potato (Nap et al., 1992. Plant Mol Biol 23:1573-1582), Lhcb3 promoter from Brassica napus (Boivin et al., 1993. Genome 36:139-46, psaD, psaF (of Photosystem I) promoters from Pfannschmidt et al., 2001. J Biol Chem 276:36125-30 and Flieger et al., 2002. Plant J 6:359-368, tobacco light-inducible ribulose 1,5-bisphosphate carboxylase small subunit (RUBISCO-SSU) (Mazur & Chua, 1985. NAR 13: 2373-2386), or rice GOSS promoter (de Pater, S., Hensgens, L. A. and Schilperoort, R. A. 1990. Plant Mol. Biol. 15 (3), 399-406 (1990), the maize phosphoenolpyruviate carboxylase (PEPC) promoter (Koziel et al, 1993. Biotechnology 11:194-200) and the like. Others include, for example, Yamamoto et al. (1997) Plant J. 12(2):255-265; Kwon et al. (1995) Plant Physiol. 105:357-67; Yamamoto et al. (1995) Plant Cell Physiol. 35(5):773-778; Gotor et al. (1993) Plant J. 3:509-18; Orozco et al. (1993) Plant Mol. Biol. 23(6):1129-1138; Baszczynski et al. (1988) Nucl. Acid Res. 16:5732; Mitra et al. (1995) Plant Molecular Biology 26:35-93; Kayaya et al. (1995) Molecular and General Genetics 258:668-675; and Matsuoka et al. (1993) Proc. Natl. Acad. Sci. USA 90(20):9586-9590. Senescence regulated promoters are also of use, such as, SAM22 (Crowell et al. (1992) Plant Mol. Biol. 18:559-566). See also U.S. Pat. No. 5,589,052, herein incorporated by reference.

A strongly or weakly constitutive plant promoter that directs expression of a polynucleotide of interest nucleic acid in all tissues of a plant can be employed. Such promoters are active under most environmental conditions and states of development or cell differentiation. In addition to the promoters mentioned above examples of constitutive promoters include the 1′- or 2′-promoter of Agrobacterium tumefaciens, and other transcription initiation regions from various plant genes known to those of skill. Where over expression of a polypeptide of interest is detrimental to the plant, one of skill will recognize that weak constitutive promoters can be used for low-levels of expression. Generally, by “weak promoter” a promoter that drives expression of a coding sequence at a low level is intended. By “low level” levels from about 1/1000 transcripts to about 1/100,000 transcripts, to about as low as 1/500,000 transcripts per cell are intended. Alternatively, it is recognized that weak promoters also include promoters that are expressed in only a few cells and not in others to give a total low level of expression. Where a promoter is expressed at unacceptably high levels, portions of the promoter sequence can be deleted or modified to decrease expression levels. In those cases where high levels of expression is not harmful to the plant, a strong promoter, e.g., a t-RNA, or other pol III promoter, or a strong pol II promoter, e.g., the cauliflower mosaic virus promoter, CaMV, 35S promoter can be used. Constitutive promoters include, for example, the Gos2 promoter (de Pater et al. 1992. Plant J 2:837-844), the core promoter of the Rsyn7 promoter and other constitutive promoters disclosed in WO 99/53838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1985) EMBO J. 3:2723-2730); ALS promoter (U.S. Pat. No. 5,659,026), and the like. Other constitutive promoters include, for example, those disclosed in U.S. Pat. Nos. 5,608,159; 5,608,155; 5,605,121; 5,569,597; 5,566,785; 5,399,680; 5,268,563; 5,608,152; and 6,177,611.

Shoot-preferred promoters include, shoot meristem-preferred promoters such as promoters disclosed in Weigal et al. (1992) Cell 69:853-859; Accession No. AJ131822; Accession No. Z71981; Accession No. AF059870, the ZAP promoter (U.S. patent application Ser. No. 10/387,937), the maize tb1 promoter (Wang et al. (1999) Nature 398:236-239, and shoot-preferred promoters disclosed in McAvoy et al. (2003) Acta Hort. (ISHS) 625:379-385.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzene sulfonamide herbicide safeners; the maize GST promoter, which is activated by hydrophobic electrophilic compounds that are used as pre-emergent herbicides; and the tobacco PR-la promoter, which is activated by salicylic acid. Chemically-inducible promoters include those induced by tetracycline (Gatz et al., 1992. Plant Journal 2:397-404), steroids (Aoyama and Chua, 1997. Plant Journal 11:605-612), estrogens (Zuo et al., 2000. Plant Journal 24:265-273), ethanol (Caddick et al., 1998. Nature Biotechnology 16:177-180), copper (Melt et al., 1993. Proc. Nat. Acad. Sci. 90:4567-4571), and safener/auxins (De Veylder et al., 1997. Plant Cell Physiol. 38:568-577). Other chemical-regulated promoters of interest include steroid-responsive promoters. See, for example, the glucocorticoid-inducible promoter in Schena et al. (1991) Proc. Natl. Acad. Sci. USA 88:10421-10425 and McNellis et al. (1998) Plant J. 14(2):247-257 and the tetracycline-inducible and tetracycline-repressible promoters for example, Gatz et al. (1991) Mol. Gen. Genet. 227:229-237, and U.S. Pat. Nos. 5,814,618 and 5,789,156, herein incorporated by reference.

A promoter may fall into none, one, or more of the above groupings and may have utility in the present invention with respect to its tissue-specificity or timing or other characteristic, or with respect to a combination of such characteristics.

In addition, the constructs may contain control regions that regulate as well as engender expression. Generally, in accordance with many commonly practiced procedures, such regions will operate by controlling transcription, such as transcription factors, repressor binding sites and termination signals, among others. For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Transcription of the DNA encoding the ccs52 polypeptides by higher eukaryotes may be increased by inserting an enhancer sequence into the vector Enhancers are cis-acting elements of DNA, usually about from 10 to 300 bp, that act to increase transcriptional activity of a promoter in a given host cell-type. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. In one aspect, a ccs52 is expressed using its native promoter in combination with an enhancer such as the 35S enhancer.

Other examples of enhancers include the SV40 enhancer, which is located on the late side of the replication origin at by 100 to 270, the cytomegalovirus early promoter enhancer, the polyoma enhancer on the late side of the replication origin, and adenovirus enhancers. Additional enhancers useful in the invention to increase transcription of the introduced DNA segment, include, inter alia, viral enhancers like those within the 35S promoter, as shown by Odell et al. (1988) Plant Mol. Biol. 10:263-72, and an enhancer from an opine gene as described by Fromm et al. (1989) Plant Cell 1:977. The enhancer may affect the tissue-specificity and/or temporal specificity of expression of sequences included in the vector.

Termination regions also facilitate effective expression by ending transcription at appropriate points. Useful terminators for practicing this invention include, but are not limited to, pinII (See An et al. (1989) Plant Cell 1(1):115-122), glb1 (See Genbank Accession #L22345), gz (See gzw64a terminator, Genbank Accession #S78780), and the nos terminator from Agrobacterium.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 5:320-335), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,850), direct gene transfer (Paszkowski et al. (1985) EMBO J. 3:2717-2722), and ballistic particle acceleration (see, for example, Sanford et al., U.S. Pat. No. 5,955,050; Tomes et al., U.S. Pat. No. 5,879,918; Tomes et al., U.S. Pat. No. 5,886,255; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:521-577; Sanford et al. (1987) Particulate Science and Technology 5:27-37 (onion); Christou et al. (1988) Plant Physiol. 87:671-675 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-325 (soybean); Datta et al. (1990) Biotechnology 8:736-750 (rice); Klein et al. (1988) Proc. Natl. Acad. Sci. USA 85:5305-5309 (maize); Klein et al. (1988) Biotechnology 6:559-563 (maize); Tomes, U.S. Pat. No. 5,250,855; Buising et al., U.S. Pat. Nos. 5,322,783 and 5,325,656; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:550-555 (maize); Fromm et al. (1990) Biotechnology 8:833-839 (maize); Hooykaas-Van Slogteren et al. (1985) Nature (London) 311:763-765; Bowen et al., U.S. Pat. No. 5,736,369 (cereals); Bytebier et al. (1987) Proc. Natl. Acad. Sci. USA 85:5355-5359 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:515-518 and Kaeppler et al. (1992) Theor. Appl. Genet. 85:560-566 (whisker-mediated transformation); D'Halluin et al. (1992) Plant Cell 5:1595-1505 (electroporation); Li et al. (1993) Plant Cell Reports 12:250-255 and Christou and Ford (1995) Annals of Botany 75:507-513 (rice); Osjoda et al. (1996) Nature Biotechnology 15:755-750 (maize via Agrobacterium tumefaciens); Leelavathi et al. (2004) Plant Cell Reports 22:465-470 (cotton via Agrobacterium tumefaciens); Kumar et al. (2004) Plant Molecular Biology 56:203-216 (cotton plastid via bombardment); all of which are herein incorporated by reference.

In specific embodiments, the ccs52 sequences employed in the invention can be provided to a plant using a variety of transient transformation methods. Such transient transformation methods include, but are not limited to, the introduction of the ccs52 protein or variants and fragments thereof directly into the plant or the introduction of the ccs52 transcript into the plant. Such methods include, for example, microinjection or particle bombardment. See, for example, Crossway et al. (1986) Mol Gen. Genet. 202:179-185; Nomura et al. (1986) Plant Sci. 55:53-58; Hepler et al. (1995) Proc. Natl. Acad. Sci. 91: 2176-2180 and Hush et al. (1995) The Journal of Cell Science 107:775-785, all of which are herein incorporated by reference. Alternatively, the ccs52 polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3153).

In other embodiments, the polynucleotide may be introduced into plants by contacting plants with a virus or viral nucleic acids. Generally, such methods involve incorporating a nucleotide construct of the invention within a viral DNA or RNA molecule. It is recognized that a ccs52 may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25855, WO99/25850, WO99/25855, and WO99/25853, all of which are herein incorporated by reference. Briefly, the polynucleotide of the invention can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-85. These plants may then be pollinated with either the same transformed strain or different strains, and the resulting progeny having desired expression of the phenotypic characteristic of interest can be identified. Two or more generations may be grown to ensure that expression of the desired phenotypic characteristic is stably maintained and inherited, and then seeds can be harvested to ensure expression of the desired phenotypic characteristic has been achieved. In this manner, the present invention provides a transformed seed (also referred to as a “transgenic seed”) having a ccs52 polynucleotide, for example, an expression cassette of the invention, stably incorporated into its genome.

The present invention may be used for transformation of any plant species, including, but not limited to, monocots and dicots. Examples of plant species of interest include, but are not limited to, corn (Zea mays, also known as maize), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In certain embodiments the ccs52 nucleic acid sequences can be stacked with any combination of polynucleotide sequences of interest in order to create plants with a desired phenotype. The combinations generated may include multiple copies of any one of the polynucleotides of interest. For example, a ccs52 polynucleotide may be stacked with any other polynucleotide(s) of the present invention. The polynucleotides of the present invention can also be stacked with any other gene or combination of genes involved in increasing yield or growth including for example, polynucleotides encoding proteins that stimulate the G1-S transition in the cell cycle, such as RepA (Gordon0Kamm et al., 2002. PNAS 99:11975-11980), CycD (Riou-Kamlichi et al. 2000. Mol. Cell. Biol. 20:4513-4521)or E2F.(De Veylder et al. 2002. EMBO J 21:1360-1368). The additional genes can be driven by the same promoter as the ccs52 polynucleotide, such as a leaf-preferred promoter, or a different promoter.

The polynucleotides employed in the present invention can also be stacked with any other gene or combination of genes to produce plants with a variety of desired trait combinations including but not limited to traits desirable for animal feed such as high oil genes (e.g., U.S. Pat. No. 6,232,529); balanced amino acids (e.g. hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122); and high methionine proteins (Pedersen et al. (1986) J. Biol. Chem. 261:6279; Kirihara et al. (1988) Gene 71:359; and Musumura et al. (1989) Plant Mol. Biol. 12: 123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser et al (1986) Gene 48:109); lectins (Van Damme et al. (1994) Plant Mol. Biol. 24:825); fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility, stalk strength, flowering time, or transformation technology traits such as cell cycle regulation or gene targeting (e.g. WO 99/61619; WO 00/17364; WO 99/25821).

These stacked combinations can be created by any method including but not limited to cross breeding plants by any conventional or TopCross methodology, or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant.

IV. Modulating the Concentration and/or Activity of a ccs52 Polypeptide

A method for modulating the concentration and/or activity of a polypeptide of the present invention in a plant is provided. In general, concentration and/or activity is increased or decreased by at least 1%, 5%, 10%, 20%, 30%, 50%, 50%, 60%, 70%, 80%, or 90% relative to a native control plant, plant part, or cell. Modulation in the present invention may occur at any desired stage of development. In specific embodiments, the polypeptides of the present invention are modulated in monocots, particularly maize. In specific embodiments, the polypeptides of the present invention are modulated in dicots, particularly soybean.

A “subject plant or plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; (d) a plant or plant cell genetically identical to the subject plant or plant cell but where the gene of interest is under control of a different promoter than the gene of interest in the subject plant or plant cell itself; or (e) the subject plant or plant cell itself, under conditions in which the gene of interest is not expressed.

The expression level of the ccs52 polypeptide may be measured directly, for example, by assaying for the level of the ccs52 polypeptide in the plant, or indirectly, for example, by measuring the ccs52 activity of the ccs52 polypeptide in the plant. Methods for determining the ccs52 activity are described elsewhere herein and include evaluation of phenotypic changes, such as increased yield or ploidy.

In specific embodiments, the ccs52 polypeptide or polynucleotide is introduced into the plant cell. Subsequently, a plant cell having the introduced sequence is selected using methods known to those of skill in the art such as, but not limited to, Southern blot analysis, DNA sequencing, PCR analysis, or phenotypic analysis. A plant or plant part altered by the foregoing embodiments is grown under plant forming conditions for a time sufficient to allow modulation of the concentration and/or activity of the ccs52 polypeptides in the plant. Plant forming conditions are well known in the art and are discussed briefly elsewhere herein.

It is also recognized that the level and/or activity of the polypeptide may be modulated by employing a polynucleotide that is not capable of directing, in a transformed plant, the expression of a protein or an RNA. For example, the employed ccs52 polynucleotides may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,985; all of which are herein incorporated by reference. See also, WO 98/59350, WO 99/07865, WO 99/25821, and Beetham et al. (1999) Proc. Natl. Acad. Sci. USA 96:8775-8778; herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may be incorporated into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprise at least one nucleotide.

A. Increasing the Activity and/or Level of a ccs52 Polypeptide

Methods are provided to increase the activity and/or level of a ccs52 polypeptide. An increase in the level and/or activity of the ccs52 polypeptide of the invention can be achieved by providing to the plant a ccs52 polypeptide. The ccs52 polypeptide can be provided by introducing the amino acid sequence of the ccs52 polypeptide into the plant, introducing into the plant a nucleotide sequence encoding a ccs52 polypeptide, or alternatively, by modifying a genomic locus encoding the ccs52 polypeptide.

As discussed elsewhere herein, many methods are known in the art for providing a polypeptide to a plant including, but not limited to, direct introduction of the polypeptide into the plant, introducing into the plant (transiently or stably) a polynucleotide construct encoding a polypeptide having ccs52 activity. It is also recognized that the methods of the invention may employ a polynucleotide that is not capable of directing, in the transformed plant, the expression of a protein or an RNA.

B. Reducing the Activity and/or Level of a ccs52 Polypeptide

Methods are provided to reduce or eliminate the activity of a ccs52 polypeptide of the invention by transforming a plant cell with an expression cassette that expresses a polynucleotide that inhibits the expression of the ccs52 polypeptide. The polynucleotide may inhibit the expression of the ccs52 polypeptide directly, by preventing transcription or translation of the ccs52 messenger RNA, or indirectly, by encoding a polypeptide that inhibits the transcription or translation of a ccs52 gene encoding ccs52 polypeptide. Methods for inhibiting or eliminating the expression of a gene in a plant are well known in the art, and any such method may be used in the present invention to inhibit the expression of the ccs52 polypeptide.

In accordance with the present invention, the expression of ccs52 polypeptide is inhibited if the protein level of the ccs52 polypeptide is less than 70% of the protein level of the same ccs52 polypeptide in a plant that has not been genetically modified or mutagenized to inhibit the expression of that ccs52 polypeptide. In particular embodiments of the invention, the protein level of the ccs52 polypeptide in a modified plant according to the invention is less than 60%, less than 50%, less than 40%, less than 30%, less than 20%, less than 10%, less than 5%, or less than 2% of the protein level of the same ccs52 polypeptide in a plant that is not a mutant or that has not been genetically modified to inhibit the expression of that ccs52 polypeptide. The expression level of the ccs52 polypeptide may be measured directly, for example, by assaying for the level of ccs52 polypeptide expressed in the plant cell or plant, or indirectly, for example, by measuring the ccs52 activity of the ccs52 polypeptide in the plant cell or plant, or by measuring the phenotypic changes in the plant. Methods for performing such assays are described elsewhere herein.

In other embodiments of the invention, the activity of the ccs52 polypeptides is reduced or eliminated by transforming a plant cell with an expression cassette comprising a polynucleotide encoding a polypeptide that inhibits the activity of a ccs52 polypeptide. The activity of a ccs52 polypeptide is inhibited according to the present invention if the ccs52 activity of the ccs52 polypeptide is less than 70% of the ccs52 activity of the same ccs52 polypeptide in a plant that has not been modified to inhibit the ccs52 activity of that ccs52 polypeptide. In particular embodiments of the invention, the ccs52 activity of the ccs52 polypeptide in a modified plant according to the invention is less than 60%, less than 50%, less than 40%, less than 30%, less than 20%, less than 10%, or less than 5% of the ccs52 activity of the same ccs52 polypeptide in a plant that that has not been modified to inhibit the expression of that ccs52 polypeptide. The ccs52 activity of a ccs52 polypeptide is “eliminated” according to the invention when it is not detectable by the assay methods described elsewhere herein. Methods of determining the alteration of ccs52 activity of a ccs52 polypeptide are described elsewhere herein.

In other embodiments, the activity of a ccs52 polypeptide may be reduced or eliminated by disrupting the gene encoding the ccs52 polypeptide. The invention encompasses mutagenized plants that carry mutations in ccs52 genes, where the mutations reduce expression of the ccs52 gene or inhibit the ccs52 activity of the encoded ccs52 polypeptide.

Thus, many methods may be used to reduce or eliminate the activity of a ccs52 polypeptide. In addition, more than one method may be used to reduce the activity of a single ccs52 polypeptide. In some embodiments of the present invention, a plant is transformed with an expression cassette that is capable of expressing a polynucleotide that inhibits the expression of a ccs52 polypeptide of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one ccs52 polypeptide is an expression cassette capable of producing an RNA molecule that inhibits the transcription and/or translation of at least one ccs52 polypeptide of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Compositions of the invention comprise sequences encoding maize seed proteins and variants and fragments thereof. Methods of the invention involve the use of, but are not limited to, transgenic expression, antisense suppression, co-suppression, RNA interference, gene activation or suppression using transcription factors and/or repressors, mutagenesis including transposon tagging, directed and site-specific mutagenesis, chromosome engineering (see Nobrega et. al., Nature 431:988-993(04)), homologous recombination, TILLING, and biosynthetic competition to manipulate, in plants and plant seeds and grains, the expression of seed proteins, including, but not limited to, those encoded by the sequences disclosed herein.

Exemplary methods for decreasing or eliminating the expression of genes include the transgenic application of transcription factors (Pabo, C. O., et al. (2001) Annu Rev Biochem 70, 313-40.; and Reynolds, L., et al (2003), Proc Natl Acad Sci USA 100, 1615-20.), and homologous recombination methods for gene targeting (see U.S. Pat. No. 6,187,994). Similarly, it is possible to eliminate the expression of a single gene by replacing its coding sequence with the coding sequence of a second gene using homologous recombination technologies (see Bolon, B. Basic Clin. Pharmacol. Toxicol. 95:4,12, 154-61 (2004); Matsuda and Alba, A., Methods Mol. Bio. 259:379-90 (2004); Forlino, et. al., J. Biol. Chem. 274:53, 37923-30 (1999)).

### V. Modulating the Yield of a Plant

Methods are provided for the use of ccs52 sequences to modulate the yield of a plant. As described elsewhere herein, the ccs52 sequences may be novel sequences of the present invention or known. In specific embodiments, methods are provided to increase yield of a plant while maintaining fertility of the plant. Typically, the ccs52 is expressed in non-germline plant cells or non-meristematic plant cells. In some examples, the ccs52 is expressed in plant cells that expresses photosynthetic genes. In some cases, the plant cells are plant cells committed to becoming differentiated plant cells having chlorophyll or differentiated plant cells having chlorophyll or combinations thereof and the ccs52 expression will not affect the plant's germline thus allowing for the production of polyploidy plants that are fertile.

Modulating the spatial and temporal expression, level and/or activity of a ccs52 sequence can maintain or increase plant yield or growth. In one method, a ccs52 nucleotide sequence is introduced into the plant and the spatial and temporal expression, level and/or activity of the ccs52 polypeptide is modulated, thereby improving the yield of the plant and maintaining fertility, which may be reflected in, for example, the biomass, ear size and seed set. Other characteristics that may be affected are described elsewhere herein. Often the introduced ccs52 nucleotide construct is stably incorporated into the genome of the plant and transmitted to progeny.

Methods to assay for a modulation in ear size or seed set or both are known to one skilled in the art. The genetically modified plant having the modulated level and/or activity of ccs52 polypeptide will have a larger ear, higher number and/or mass of developing seed than a wild type (non-transformed) plant.

Accordingly, the present invention further provides plants having increased yield and maintained fertility. In some embodiments, the plants having increased yield and maintained fertility have a modulated level/activity of a ccs52 polypeptide of the invention or known ccs52. In some examples, the plant comprises a ccs52 nucleotide sequence operably linked to a suitable promoter that does not drive expression in germline plant cells or in meristematic plant cells or effectively express ccs52 in germline plant cells or in meristematic plant cells. In some examples, the plant comprises a ccs52 nucleotide sequence operably linked to a suitable promoter the drives expression in plant cells committed to becoming differentiated plant cells having chlorophyll, differentiated plant cells having chlorophyll, e.g., cells in a leaf, or both. In certain embodiments, such plants have stably incorporated into their genome a nucleic acid molecule comprising a ccs52 nucleotide sequence of the invention operably linked to a promoter that drives expression in the plant cell.

### VI. Modulating Shoot and Leaf Development

Methods are also provided for modulating shoot and leaf development in a plant. By “modulating shoot development” and/or “modulating leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length, and leaf senescence. As used herein, “leaf development” and “shoot development” encompass all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner et al. (2001) PNAS 98:10587-10592 and U.S. Application No. 2003/0075698, each of which is herein incorporated by reference.

The method for modulating shoot and/or leaf development in a plant comprises modulating the activity and/or level of a ccs52 polypeptide, for example a ccs52 of the invention or known ccs52. In one embodiment, a ccs52 sequence of the invention is provided. In other embodiments, the ccs52 nucleotide sequence can be provided by introducing into the plant a polynucleotide comprising a ccs52 nucleotide sequence, expressing the ccs52 sequence in a plant cells committed to becoming differentiated plant cells having chlorophyll, differentiated plant cells having chlorophyll or both, and thereby modifying shoot and/or leaf development. In other embodiments, the ccs52 nucleotide construct introduced into the plant is stably incorporated into the genome of the plant.

In specific embodiments, shoot and/or leaf development is modulated by modulating the level and/or activity of the ccs52 in the plant. A modulation in ccs52 activity can result in at least one or more of the following alterations in shoot and/leaf development including, but not limited to, altered (increased or decreased) shoot growth, altered photosynthesis, modulated leaf number, altered leaf surface, altered length of internodes, and modulated leaf senescence. Modulating the level of the ccs52 polypeptide in the plant can thereby increase plant yields.

As discussed above, one of skill will recognize the appropriate promoter to use to modulate shoot and leaf development of the plant. Exemplary promoters for this embodiment include constitutive promoters or promoters that are preferentially active in photosynthetic tissues including, for example, shoot-preferred promoters, shoot meristem-preferred promoters, and leaf-preferred promoters. Exemplary promoters have been disclosed elsewhere herein.

Accordingly, the present invention further provides plants having a modulated shoot and/or leaf development when compared to a control plant. In some embodiments, the plant of the invention has an increased level/activity or a decreased level/activity of a ccs52 polypeptide of the invention or known ccs52.

It is further recognized that increasing seed size and/or weight can be accompanied by an increase in the rate of growth of seedlings or an increase in early vigor. In addition, modulating the plant's yield, as discussed above, along with modulation of leaf development can increase plant yield and vigor. As used herein, the term “vigor” refers to the relative health, productivity, and rate of growth of the plant and/or of certain plant parts, and may be reflected in one or more various developmental attributes, such as concentration or level of chlorophyll, photosynthetic rate, total biomass, and root biomass. Of particular relevance is the ability of a plant to grow rapidly during early development, and relates to the successful establishment, after germination, of a well-developed root system and a well-developed photosynthetic apparatus. Improvements in vigor are measured with reference to a control as defined elsewhere herein.

### VII. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development, or radial expansion.

The methods for modulating root development comprise modulating (reducing or increasing) the level and/or activity of the ccs52 polypeptide in the plant. In one method, a ccs52 nucleotide sequence is introduced into the plant and the level and/or activity of the ccs52 polypeptide is modulated. In other methods, the ccs52 nucleotide construct introduced into the plant is stably incorporated into the genome of the plant.

A modulation in ccs52 activity can result in at least one or more of the following alterations to root development, including, but not limited to, larger root meristems, increased root growth, enhanced radial expansion, an enhanced vasculature system, increased root branching, more adventitious roots, and/or increased fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc. Methods of measuring such developmental alterations in the root system are known in the art. See, for example, U.S. Application No. 2003/0075698 and Werner et al. (2001) PNAS 18:10587-10592, both of which are herein incorporated by reference.

As discussed above, one of skill will recognize the appropriate promoter to use to modulate root development in the plant. Exemplary promoters are disclosed elsewhere herein.

Stimulating root growth and increasing root mass by modulating the activity and/or level of the polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by modulating the level and/or activity of the ccs52 polypeptide also finds use in promoting in vitro propagation of explants.

Accordingly, the present invention further provides plants having modulated root development when compared to the root development of a control plant. In some embodiments, the plant of the invention has a modulated level/activity of the ccs52 polypeptide and has enhanced root growth and/or root biomass. In some embodiments, such plants have stably incorporated into their genome a nucleic acid molecule comprising a ccs52 nucleotide sequence operably linked to a promoter that drives expression in plant cells committed to becoming differentiated plant cells having chlorophyll, such as a dividing leaf cell-preferred promoter or differentiating mesophyll cell-preferred promoter, wherein expression of the sequence modulates the level and/or activity of the ccs52 polypeptide. In other embodiments, such plants have stably incorporated into their genome a nucleic acid molecule comprising a ccs52 nucleotide sequence operably linked to a promoter that drives expression in the differentiated plant cell having chlorophyll, such as a leaf-preferred promoter, wherein expression of the sequence modulates the level and/or activity of the ccs52 polypeptide.

### VIII. Sequences

Sequences of Zm ccs52:
>ZM-ccs52 (maize ccs52 cDNA
(SEQ ID NO: 1)
atggacgcaggctcccgctcgatctcctcggcgaagaaccgcgccgcc
gccgtcgccgccgcgccccggccaccgctgcaggaggcgggctcccgc
ccctacatgccatcgctgagctcgggaccccgcaacccgtcggccaag
tgctacggcgacaggttcatcccggacaggtcggcgatggacatggac
ttggcgcactacttgatactgagcccaggagggacaaggagaacgcgt
cgggcatggcggcgtccccgtccaaggaggcgtaccggaggctgctcg
cggagaagctgctcaacaaccggacacggatcctcgctttcaggagca
agccgccggagcccgagaacgtttcttttgcggacacgacttcctcca
acctgcaggccaagccggccaaacagcggcgccacattccccagtctg
ccgagaggaccctagacgcaccagagctagttgacgactactacctca
acttgatgactggggaagcaacaacgtgctatccattgattgggagac
acggtgtacctgtgggatgcatcgagcggatccacatccgagcttgtg
accgtcggcgaggacagtggtcctgttacaagcgttagctgggctcct
gatggtcggcacatggccgtcgggctcaactcgtctgacgtccagctc
tgggacaccagctccaaccgactgttgagaacactcagaggtgcgcat
gaggcaagggtaggctcgctggcatggaacaacagcgtcttgaccacc
ggttgcatggacggcaagatcgtgaacaatgacgtaaggattagagac
cacgtcgtgcagaggtacgaggggcacagccaggaggtctgcgggctc
aagtggtccggatcagggcagcagctggccagcggaggcaacgacaac
cttctgcacatttgggatgtgtcgatggcatcatccatgccatctgct
ggccgcaaccagtggctgcataggctcgaggaccacatggccgccgtg
aaggcactcgcgtggtgcccgttccagagcaacttgctggccaccggc
ggcggtggcagcgaccgctgcatcaagttctggaacacgcacaccggt
gtgtgcctgaactcggttgataccggatcacaggtgtgcgctctgctg
tggaacaagaacgagagggagctgctgagctcacatggattcacacag
aaccaactcaccttgtggaagtacccatcgatggtgaagatggctgaa
cttaatggccatacctctcgcgtcctcttcatggctcagagtcctgat
ggatgcacagtagcgtcggctgctgctgacgagaccctccggttctgg
aacgtttttggaacccctgaaacgcccaagcctgcagccaaagatccc
acactgggatgttcaacagcttcaaacatatccgatag
>Zm-ccs52 (maize ccs52 amino acid)
(SEQ ID NO: 2)
MDAGSRSISSAKNRAAAVAAAPRPPLQEAGSRPYMPSLSSGPRNPSAK
CYGDRFIPDRSAMDMDLAHYLLTEPRRDKENASGMAASPSKEAYRRLL
AEKLLNNRTRILAFRSKPPEPENVSFADTTSSNLQAKPAKQRRHIPQS
AERTLDAPELVDDYYLNLLDWGSNNVLSIALGDTVYLWDASSGSTSEL
VTVGEDSGPVTSVSWAPDGRHMAVGLNSSDVQLWDTSSNRLLRTLRGA
HEARVGSLAWNNSVLTTGCMDGKIVNNDVRIRDHVVQRYEGHSQEVCG
LKWSGSGQQLASGGNDNLLHIWDVSMASSMPSAGRNQWLHRLEDHMAA
VKALAWCPFQSNLLATGGGGSDRCIKFWNTHTGVCLNSVDTGSQVCAL
LWNKNERELLSSHGFTQNQLTLWKYPSMVKMAELNGHTSRVLFMAQSP
DGCTVASAAADETLRFWNVFGTPETPKPAAKASHTGMFNSFKHIR
Sequences of sorghum ccs52:
>sorghum-ccs52 (sorghum ccs52 cDNA)
(SEQ ID NO: 3)
atggacgcaggatcccactcgatctcctcggagaagagccgcgccgcc
gccgcgccccggccgccgctgcaggaggcggtctcccgcccctacatg
ccatcgctgggctcgggatgccgtaacccgtcggccaagtgctacggc
gacagattcatcccggacagatcggcgatggacatggacatggcacac
ttcctgctcactgagcccaggaaggacaaggagaacgcggcggcgtcc
ccgtccaaggaggcgtaccggaggctgctcgcggagaagctgctcaac
aaccggacacggatcctcgccttcaggaacaagccgccggagcccgag
aacgtatctttcgccgatgcggcttcctccaacctgcaggccaagcct
gctaagcagcggcgccacattccccagtctgccgagaggaccctagac
gccccagagcttgttgatgactactacctcaacctgcttgactggggg
agcaacaatgtgctgtccattgctctgggagacacactgtacctgtgg
gatgcgtcgagtggatccacatccgagatgtgaccatcgatgaggaca
gcggtcctattaccagtgttagctgggctcctgatggtcggcacatcg
ccgtggggctcaactcgtccgacgtccagattgggacaccagctccaa
ccgactgttgagaacactcagaggtgtgcatgaggcaagggtaggttc
actggcatggaacaacagcatcctaaccaccggtggcatggatggcaa
gattgtgaacaatgacgtgaggattagagaccacgttgtgcagactta
cgaggggcacagccaggaggtgtgcgggctcaagtggtctggatcagg
gcagcagctggccagcggaggcaacgacaaccttctgcacatttggga
tgtgtcgatggcatcatccatgccatctgctggccgcaaccagtggct
acataggctcgaggaccacacggccgccgtgaaggcactcgcgtggtg
cccgttccagagcaacttgatgccactggcggtggtggcagcgatcgt
tgcatcaagttctggaacacacacactggtgcgtgcctgaactcagtt
gacaccggatcacaggtgtgcgctatctctggaacaagaatgagaggg
agctgctgagttcac atggattcacacagaaccaactgactttgtgg
aagtacccatcgatggtgaagatggctgaacttactggccatacctct
cgtgtccttttcatggctcagagtcctgatggatgcacagtagcgtca
gctgctgcagatgagaccctccggttctggaacgtttttggagcccct
gaagcgcccaagcctgctgccaaagcttcccacactgggatgttcaac
agcttcaaccatatccgatag
>sorghum-ccs52 (sorghum ccs52 amino acid)
(SEQ ID NO: 4)
MDAGSHSISSEKSRAAAAPRPPLQEAVSRPYMPSLGSGCRNPSAKCYG
DRFIPDRSAMDMDMAHFLLTEPRKDKENAAASPSKEAYRRLLAEKLLN
NRTRILAFRNKPPEPENVSFADAASSNLQAKPAKQRRHIPQSAERTLD
APELVDDYYLNLLDWGSNNVLSIALGDTLYLWDASSGSTSELVTIDED
SGPITSVSWAPDGRHIAVGLNSSDVQLWDTSSNRLLRTLRGVHEARVG
SLAWNNSILTTGGMDGKIVNNDVRIRDHVVQTYEGHSQEVCGLKWSGS
GQQLASGGNDNLLHIWDVSMASSMPSAGRNQWLHRLEDHTAAVKALAW
CPFQSNLLATGGGGSDRCIKFWNTHTGACLNSVDTGSQVCALLWNKNE
RELLSSHGFTQNQLTLWKYPSMVKMAELTGHTSRVLFMAQSPDGCTVA
SAAADETLRFWNVFGAPEAPKPAAKASHTGMFNSFNHIR
Sequences of soybean ccs52:
>Gm-ccs52-1 (soybean ccs52 cDNA)
(SEQ ID NO: 5)
atggaggactcgtccggccacctgaatattcctccggccgccgcggcg
gcgactctccggcacgttgaccgcatgatcaactccaaccactacacc
tcgccttccagaacaatctactccgaccgcttcattcccagcagatct
gcctcgaaattcgcgctatcgacatcgcctggcctcccgggggcggcg
acgacagctccagcgcctacaccacgctcctccgcaccgcgctcttcg
gccccgacatcgagccgccgcactcgccggcgatgactctccccagcc
ggaatatcttccgttacaaaaccgagacgcgccagtccatgcactcgc
actcgccgttatgtgcgacgattcggtccccggcgttgtccacggccc
ggtcaaggctccgaggaaggttccgaggtcgccttttaaggttctgga
tgcgcctgcgctgcaagatgatttttacctgaatcttgtggattggtc
ttcgcataatgtgttggctgttggtctgggaaactgtgtttatctctg
gaatgcttgtagcagcaaggttactaaattatgtgacttggggattga
tgacctcgtttgttcggttggctgggctcagcgtggtacacaccttgc
tgttggaactagcaatggtaaagttcagatttgggatgcatctcgatg
caagaagataagatctatggagggtcatcggttacgggttgggacctt
ggcttggagttcatctatttgtatctggcggcagggataagaatattt
atcaaagagatatccgtgcacaagaagattttgtcagtaaattgtcag
ggcacaaatcagaggtttgtggactgaagtggtcttatgataaccgtg
agttggcatctggaggaaatgacaacagattgtttgtttggaatcaac
actcaactcagcctgtcctgaagtactgtgagcatacagcagctgtta
aagctattgcatggtctcctcatcttcacggacttcttgcatctgggg
gaggaactgcagaccgatgcatacgtttctggaatacaaccacaaact
cacatttaagctgcatggacacgggaagtcaggtttgcaatcttgtct
ggtccaaaaatgtcaatgaactagtaagcacgcatggctattcccaga
accagataattgtttggagatacccctccatgtcaaagttggccactc
ttacgggtcatacctacagagttctttatcttgccatttctccggatg
gacagactattgtaactggagctggtgatgaaacacttaggttctgga
acgtattcccttcccctaaatcacagaatactgatagtgaaatcggag
catcatcttttggaagaacaattattaggtga
>Gm-ccs52-1 (soybean ccs52 amino acid)
(SEQ ID NO: 6)
MEDSSGHLNIPPAAAAATLRHVDRMINSNHYTSPSRTIYSDRFIPSRS
ASKFALFDIAWPPGGGDDSSSAYTTLLRTALFGPDIEPPHSPAMTLPS
RNIFRYKTETRQSMHSHSPFLCDDSVPGVVHGPVKAPRKVPRSPFKVL
DAPALQDDFYLNLVDWSSHNVLAVGLGNCVYLWNACSSKVTKLCDLGI
DDLVCSVGWAQRGTHLAVGTSNGKVQIWDASRCKKIRSMEGHRLRVGT
LAWSSSLLSSGGRDKNIYQRDIRAQEDFVSKLSGHKSEVCGLKWSYDN
RELASGGNDNRLFVWNQHSTQPVLKYCEHTAAVKAIAWSPHLHGLLAS
GGGTADRCIRFWNTTTNSHLSCMDTGSQVCNLVWSKNVNELVSTHGYS
QNQIIVWRYPSMSKLATLTGHTYRVLYLAISPDGQTIVTGAGDETLRF
WNVFPSPKSQNTDSEIGASSFGRTIIR
>Gm-ccs52-2 (soybean ccs52 cDNA)
(SEQ ID NO: 7)
atggaggacttgtccggccacctgaatattcctccggccgcctccgcg
gcgactctccgccacgtggaccgcatgatcaactccaaccactacacc
tcgccttccaggacaatctactccgaccgcttcattcccagcagatct
gcctcgaaattcgcactcttcaacatcgatcgccgcccgagggccgcg
acgacagaccagtgcctacaccacgctcctccgcaccgcgctcttcgg
ccccgacttcgcgccgccgcccacgccggagaaaacggcctcgccggc
gatgacgctccccagccgaaatattttccggtacaagacagagacgcg
ccagtccatgcactcgctctcgccattcatgtgcgaggattcggtgcc
cggcgttgttcacggtccggtcaaggctccgaggaaggttccgaggtc
gccttttaaggttctggatgcgcctgcgctgcaagacgatttctacct
gaatcttgtggattggtcttcgcataatgtgttggctgttggtctggg
aaactgtgtttatctttggaatgcttgtagcagcaaggttactaaatt
atgtgacttggggattgatgaccttgtttgttcggttggctgggctca
gcgtggtacacaccttgctgttggaacaagcaatggtaaagttcagat
ttgggatgcatacgatgcaagaagataagatactggagggtcatcggt
tacgtgttggggccttggcttggagttcatctatttgtatctggtggc
agggataagaatatttatcaaagagatatccgtgcacaagaagatttt
gtcagtaaattatcagggcacaaatcagaggtttgtggactgaagtgg
tcttatgataaccgtgagttggcatctggaggaaatgacaacagattg
tttgtttggaatcaacactcaactcagcctgtcctaaagtactgtgag
catacagcagagttaaagctattgcatggtctcctcatcttcatggac
ttcttgcatctgggggaggaactgcagaccgatgcatacgtttctgga
atacaaccacaaactcacacttaagctgcatggacactggaagccagg
tttgcaatcttgtctggtccaaaaatgtcaatgaactagtaagtacac
atggctattcccagaatcagataattgtttggagataccccaccatgt
caaagttggccactcttacaggccatacctatagagttattatctagc
catttctcccgatggacagactattgtaactggagaggagatgaaaca
cttaggttctggaacgtattccatcccctaaatcacagaatactgata
gtgaaatcggagcatcatctcttggaagaacaattattaggtga
>Gm-ccs52-2 (soybean ccs52 amino acid)
(SEQ ID NO: 8)
MEDLSGHLNIPPAASAATLRHVDRMINSNHYTSPSRTIYSDRFIPSRS
ASKFALFNIASPPEGRDDSSSAYTTLLRTALFGPDFAPPPTPEKTASP
AMTLPSRNIFRYKTETRQSMHSLSPFMCEDSVPGVVHGPVKAPRKVPR
SPFKVLDAPALQDDFYLNLVDWSSHNVLAVGLGNCVYLWNACSSKVTK
LCDLGIDDLVCSVGWAQRGTHLAVGTSNGKVQIWDASRCKKIRSLEGH
RLRVGALAWSSSLLSSGGRDKNIYQRDIRAQEDFVSKLSGHKSEVCGL
KWSYDNRELASGGNDNRLFVWNQHSTQPVLKYCEHTAAVKAIAWSPHL
HGLLASGGGTADRCIRFWNTTTNSHLSCMDTGSQVCNLVWSKNVNELV
STHGYSQNQIIVWRYPTMSKLATLTGHTYRVLYLAISPDGQTIVTGAG
DETLRFWNVFPSPKSQNTDSEIGASSLGRTIIR
>Gm-ccs52-3 (soybean ccs52 cDNA)
(SEQ ID NO: 9)
atggacgaatcattcactccagcaccaccacctcctccaatgtccctt
tctcggcacgatcacgtccaacgaatgataaactcgaagcgctacaag
tcaccttcgaaaacaatatactccgacaggttcattccgagcagatcc
ggttccaatttcgatctcttcaatctaccttcgccgtcgtcgtcagag
gacagttgcagttgcagcccctacagcaccgcgctgcggagggccttg
ttcggaccagacactcccgataaatttgaaagccctaatatattccgt
tacaaaacggagactcgaaagtctatgtattactctcacccacccatt
tacttcccaggatgatatctccctggttatgacaacaatcataaacct
cccaagcgtcctcgcaagattcctccctcctcttttaaggttttggac
gcccctgcgctgcaagacgatttttatctgaatctcgtggattggtca
tccaacaatatcttggctgtggactggagaactctgtttatttgtgga
atgatctagcagcaaggtaactaaattatgcgatttggggattgacga
ttcagtttgttcagttggctgggctccacttggtacctacctgtctgt
tggatcaaacagtggtaaagtccagatttgggatgtatctcaaggcaa
gtcaataagaactatggagggtcatcgtttacgtgttggggccttggc
ttggagttcctacttttgtcttctggtggccgggataaaagcatttat
caacgagatatacgtgcacaggaggattttgtcagtaaactgtctggg
cacaagtcagaggtttgtggactgaagtggtcttatgacaaccgtgag
ctagcatctggaggaaatgacaacaggttgcttgtttggaatcaaaag
tcaacccagcccgttctgaagttctgtgagcatacagcagagttaaag
ctattgcatggtacctcatgtaaatggacttcttgcatctggaggagg
aactgtggaccgaaacattcgcttttggaatacaaccacaaactcaca
gttaaactgtatcgacactggtagtcaggtttgtaaccttgtttggtc
taaaaatgtgaatgaactcgtaagcacacatggttactcccagaacca
gataatagtttggaaatatcccaccatgtcaaagctagcaacgcttac
aggccatacttacagagttattatcttgccatatctcctgacgggcag
actatcgtcactggagaggagatgaaactcttaggttctggaatgtat
tccatcgcggaaatcacagaatactgagagtgaaattggagcttcatc
ttttggcagaactatcatcagatga
>Gm-ccs52-3 (soybean ccs52 amino acid)
(SEQ ID NO: 10)
MDESFTPAPPPPPMSLSRHDHVQRMINSKRYKSPSKTIYSDRFIPSRS
GSNFDLFNLPSPSSSEDSCSCSPYSTALRRALFGPDTPDKFESPNIFR
YKTETRKSMYSLSPTPFTSQDDLLPGYDNNHKPPKRPRKIPPSSFKVL
DAPALQDDFYLNLVDWSSNNILAVALENSVYLWNASSSKVTKLCDLGI
DDSVCSVGWAPLGTYLSVGSNSGKVQIWDVSQGKSIRTMEGHRLRVGA
LAWSSSLLSSGGRDKSIYQRDIRAQEDFVSKLSGHKSEVCGLKWSYDN
RELASGGNDNRLLVWNQKSTQPVLKFCEHTAAVKAIAWSPHVNGLLAS
GGGTVDRNIRFWNTTTNSQLNCIDTGSQVCNLVWSKNVNELVSTHGYS
QNQIIVWKYPTMSKLATLTGHTYRVLYLAISPDGQTIVTGAGDETLRF
WNVFPSRKSQNTESEIGASSFGRTIIR
>Gm-ccs52-4 (soybean ccs52 cDNA)
(SEQ ID NO: 11)
atggacgaatcattcactccaatgtcgtatttcaacatgaccacgtcc
aacgattgataaagtcgaaccgctacaagtcaccttccaaaacaatct
actccaacaggttcattcctagcagatccggttccaattttgatttct
tcaatctacctccgtcgtcctcgtcagaggacagttgcagttgcagtc
cctacagcaccgcgctgcgaagtgccttgttcggaccagacactcccg
ataaatttgaaagccctaatatattccgttacaaaacggagactcgaa
agtccttgtattactctcacccaccccattactttccaggatgacctt
ctccctggttatgaccacaatcaaaaacctcctaagcgtcctcgcaag
attcctccttcgtctittaaggttttggacgcccctgccctgcaagac
gatttttatctgaatctcgtggattggtcctccaacaatgtcttggct
gtggctctggagacctctgtttatttgtggaatgatctagcagcaagg
taactaaattatgtgatttggggattgacaactcagtttgttcggttg
gctgggctccacttggtacctacctggctgttggatcaaacagtggta
aagtccagatttgggatgtatctcaaggcaagtcaataagaactatgg
agggtcatcgtttacgtgttggagattggcctggagttcttctctttt
gtcttctggtggccgggataaaagtatttatcaacgagatattcgtgc
acaggaggatttcatcagtaaactgtctggacacaagtcagaggtctg
tggactgaagtggtcttgtgataaccgtgagctagcatccggaggaaa
tgacaacaggttgcttgtttggaatcaaaagtcaacccagcccgtcct
gaagttctgtgagcatacagcagagttaaagctattgcgtggtacctc
atgtaagtggacttcttgcatctggaggaggaactgcggaccgaaaca
ttcgattttggaatacaaccacaaacacacagttaaactgtatcgaca
ctggtagtccaggtttgtaaccttgtttggtctaaaaatgtgaatgaa
cttgtaagcacacatggttactcccagaaccagataatagtttggaaa
taccccaccatgtcaaagctagcaactcttacaggccatacttacaga
gttattatcttgccatatctcctgatggacagactatcgtcagtgggg
ctggagacgaaactcttaggttttgggatgtattccattgcagaaatc
acggaataccgagagtgaaattggtgcatcttttgggagaactatcat
tagatga
>Gm-ccs52-4 (soybean ccs52 amino acid)
(SEQ ID NO: 12)
MDESFTPMSSFQHDHVQRLIKSNRYKSPSKTIYSNRFIPSRSGSNFDF
FNLPPSSSSEDSCSCSPYSTALRSALFGPDTPDKFESPNIFRYKTETR
KSLYSLSPTPFTFQDDLLPGYDHNQKPPKRPRKIPPSSFKVLDAPALQ
DDFYLNLVDWSSNNVLAVALETSVYLWNASSSKVTKLCDLGIDNSVCS
VGWAPLGTYLAVGSNSGKVQIWDVSQGKSIRTMEGHRLRVGALAWSSS
LLSSGGRDKSIYQRDIRAQEDFISKLSGHKSEVCGLKWSCDNRELASG
GNDNRLLVWNQKSTQPVLKFCEHTAAVKAIAWSPHVSGLLASGGGTAD
RNIRFWNTTTNTQLNCIDTGSQVCNLVWSKNVNELVSTHGYSQNQIIV
WKYPTMSKLATLTGHTYRVLYLAISPDGQTIVSGAGDETLRFWDVFPL
QKSRNTESEIGASFGRTIIR

### EXPERIMENTAL EXAMPLES

The present invention is further illustrated in the following examples, in which parts and percentages are by weight and degrees are Celsius, unless otherwise stated. It should be understood that these examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Furthermore, various modifications of the invention in addition to those shown and described herein will be apparent to those skilled in the art from the foregoing description. Such modifications are also intended to fall within the scope of the appended claims. The following examples are offered by way of illustration and not by way of limitation.

### Example 1

### Maize Transformation with the Sequences of the Invention

Immature maize embryos from greenhouse donor plants are bombarded with a plasmid containing an expression cassette ccs52, as detailed in methods described elsewhere herein. The ccs52 polynucleotide is operably linked to a leaf-specific promoter (such as CAB) and, if desired, a selectable marker gene such as PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 5 hours and then aligned within the 2.5 cm target zone in preparation for bombardment.

A plasmid vector comprising the maize ccs52 sequence operably linked to a RB-CAB promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #5 in a Biorad PDS-1000 Biolistics Particle Delivery System. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-5 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored under various stress conditions and compared to control plants. Alterations in phenotype, such as improved tolerance to stress, will be monitored.

Bombardment medium (560Y) comprises 5.0 g/l N6 basal salts (SIGMA C-1516), 1.0 ml/l Eriksson's Vitamin Mix (1000× SIGMA-1511), 0.5 mg/l thiamine HCl, 120.0 g/l sucrose, 1.0 mg/12,5-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 5.0 g/l N6 basal salts (SIGMA C-1516), 1.0 ml/l Eriksson's Vitamin Mix (1000× SIGMA-1511), 0.5 mg/l thiamine HCl, 30.0 g/l sucrose, and 2.0 mg/l 2,5-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 5.3 g/l MS salts (GIBCO 11117-075), 5.0 ml/l MS vitamins stock solution (0.100 g nicotinic acid, 0.02 g/l thiamine HCL, 0.10 g/l pyridoxine HCL, and 0.50 g/l glycine brought to volume with polished D-I H₂O) (Murashige and Skoog (1962) Physiol. Plant. 15:573), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 5.3 g/l MS salts (GIBCO 11117-075), 5.0 ml/l MS vitamins stock solution (0.100 g/l nicotinic acid, 0.02 g/l thiamine HCL, 0.10 g/l pyridoxine HCL, and 0.50 g/l glycine brought to volume with polished D-I H₂O), 0.1 g/l myo-inositol, and 50.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 2

### Modulating Plant Yields

For Agrobacterium-mediated transformation of maize with the ccs52 nucleotide sequence (SEQ ID NO: 1) operably linked to a RB-CAB promoter, or a leaf-preferred promoter, the method of Zhao is employed (U.S. Pat. No. 5,981,850, and PCT patent publication WO98/32326; the contents of which are hereby incorporated by reference). Briefly, immature embryos are isolated from maize and the embryos contacted with a suspension of Agrobacterium, where the bacteria are capable of transferring the ccs52 nucleotide sequence to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 5: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

The plants are monitored for a modulation in shoot growth, leaf senescence, and/or photosynthesis when compared to an appropriate control plant. A modulation in plant yield is also monitored.

### Example 3

### Soybean Transformation

Soybean embryos are bombarded with a plasmid containing the ccs52 sequence operably linked to a CAB promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 5,955,050). A DuPont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the ccs52 operably linked to the RB-CAB promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 500 μl 70% ethanol and resuspended in 50 μl of anhydrous ethanol. The DNA/particle suspension can be sonicated three times for one second each. Five microliters of the DNA-coated gold particles are then loaded on each macro carrier disk.

Approximately 300-500 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 4

### Sunflower Meristem Tissue Transformation

Sunflower meristem tissues are transformed with an expression cassette containing the ccs52 (SEQ ID NO: 1) operably linked to a RB-CAB or a leaf-preferred promoter as follows (see also European Patent No. EP 0 586233, herein incorporated by reference, and Malone-Schoneberg et al. (1995) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 573-597), Shepard's vitamin additions (Shepard (1980) in Emergent Techniques for the Genetic Improvement of Crops (University of Minnesota Press, St. Paul, Minn.), 50 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney et al. (1992) Plant Mol. Biol. 18:301-313). Thirty to forty explants are placed in a circle at the center of a 60×20 mm plate for this treatment. Approximately 5.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the RR6 gene operably linked to the Zea mays ubiquitin promoter is introduced into Agrobacterium strain EHA105 via freeze-thawing as described by Holsters et al. (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.5 to 0.8. The Agrobacterium cells are pelleted and resuspended at a final OD₆₀₀ of 0.5 in an inoculation medium comprised of 12.5 mM MES pH 5.7, 1 gm/l NH₅Cl, and 0.3 gm/l MgSO₅.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 375B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 375B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for ccs52 activity.

NPTII-positive shoots are grafted to PIONEER® hybrid 6550 in vitro-grown sunflower seedling rootstock. Surface sterilized seeds are germinated in 58-0 medium (half-strength Murashige and Skoog salts, 0.5% sucrose, 0.3% Gelrite® pH 5.6) and grown under conditions described for explant culture. The upper portion of the seedling is removed, a 1 cm vertical slice is made in the hypocotyl, and the transformed shoot inserted into the cut. The entire area is wrapped with PARA film® to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by ccs52 activity analysis of leaf extracts while transgenic seeds harvested from NPTII-positive T₀ plants are identified by ccs52 activity analysis of small portions of dry seed cotyledon.

### Example 5

### Rice Transformation

One method for transforming DNA into cells of higher plants that is available to those skilled in the art is high-velocity ballistic bombardment using metal particles coated with the nucleic acid constructs of interest (see Klein et al. Nature (1987) (London) 327:70-73, and see U.S. Pat. No. 4,945,050). A Biolistic PDS-1000/He (BioRAD Laboratories, Hercules, Calif.) is used for these complementation experiments.

The bacterial hygromycin B phosphotransferase (Hpt II) gene from Streptomyces hygroscopicus that confers resistance to the antibiotic may be used as the selectable marker for rice transformation. In the vector, the Hpt II gene may be engineered with the 35S promoter from Cauliflower Mosaic Virus and the termination and polyadenylation signals from the octopine synthase gene of Agrobacterium tumefaciens. For example, see the description of vector pML18 in WO 97/47731, published on Dec. 18, 1997, the disclosure of which is hereby incorporated by reference.

Embryogenic callus cultures derived from the scutellum of germinating rice seeds serve as source material for transformation experiments. This material is generated by germinating sterile rice seeds on a callus initiation media (MS salts, Nitsch and Nitsch vitamins, 1.0 mg/12,4-D and 10 μM AgNO₃) in the dark at 27-28° C. Embryogenic callus proliferating from the scutellum of the embryos is transferred to CM media (N6 salts, Nitsch and Nitsch vitamins, 1 mg/12,4-D, Chu et al., 1985, Sci. Sinica 18: 659-668). Callus cultures are maintained on CM by routine sub-culture at two-week intervals and used for transformation within 10 weeks of initiation.

Callus is prepared for transformation by subculturing 0.5-1 0 mm pieces approximately 1 mm apart, arranged in a circular area of about 4 cm in diameter, in the center of a circle of Whatman #541 paper placed on CM media. The plates with callus are incubated in the dark at 27-28° C. for 3-5 days. Prior to bombardment, the filters with callus are transferred to CM supplemented with 0.25 M mannitol and 0.25 M sorbitol for 3 hr in the dark. The petri dish lids are then left ajar for 20-45 minutes in a sterile hood to allow moisture on tissue to dissipate.

Each genomic DNA fragment is co-precipitated with pML18 (containing the selectable marker for rice transformation) onto the surface of gold particles. To accomplish this, a total of 10 μg of DNA at a 2:1 ratio of trait:selectable marker DNAs are added to 50 μl aliquot of gold particles that have been resuspended at a concentration of 60 mg ml⁻¹. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are washed twice with 1 ml of absolute ethanol and then resuspended in 50 μl of absolute ethanol and sonicated (bath sonicator) for one second to disperse the gold particles. The gold suspension is incubated at −70° C. for five minutes and sonicated (bath sonicator) if needed to disperse the particles. Six μl of the DNA-coated gold particles are then loaded onto Mylar® macrocarrier disks and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen and the callus is bombarded two times. Two to four plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/l hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipette. Three ml aliquots of the callus suspension are plated onto fresh SM media and the plates are incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% Gelrite®+50 ppm hyg B) for 2 weeks in the dark at 25° C. After 2 weeks the callus is transferred to RM2 media (MS salts, Nitsch and Nitsch vitamins, 3% sucrose, 0.4% Gelrite®+50 ppm hyg B) and placed under cool white light (˜40 μEm⁻² s⁻¹) with a 12 hr photoperiod at 25° C. and 30-40% humidity. After 2-4 weeks in the light, callus begin to organize, and form shoots. Shoots are removed from surrounding callus/media and gently transferred to RM3 media (½×MS salts, Nitsch and Nitsch vitamins, 1% sucrose+50 ppm hygromycin B) in PHYTATRAYS™ (Sigma Chemical Co., St. Louis, Mo.) and incubation is continued using the same conditions as described in the previous step.

Plants are transferred from RM3 to 4″ pots containing Metro mix 350 after 2-3 weeks, when sufficient root and shoot growth have occurred.

### Example 6

### Variants of ccs52

A. Variant Nucleotide Sequences of ccs52 (SEQ ID NOS: 1, 3, 5, 7, 9, and 11) that Do Not Alter the Encoded Amino Acid Sequence

The ccs52 nucleotide sequences set forth in SEQ ID NO: 1 may be used to generate variant nucleotide sequences having 0%, 75%, 80%, 81%, 82%, 83%, 84%, 85%, 86%, 87%, 88%, 89%, 90%, 91%, 92%, 93%, 94%, 95%, 96%, 97%, 98% or 99% nucleotide sequence identity when compared to the starting unaltered ORF nucleotide sequence of SEQ ID NO: 1, 3, 5, 7, 9, or 11. These functional variants are generated using a standard codon table. While the nucleotide sequence of the variant is altered, the amino acid sequence encoded by the open reading frame does not change.

B. Variant Amino Acid Sequences of ccs52

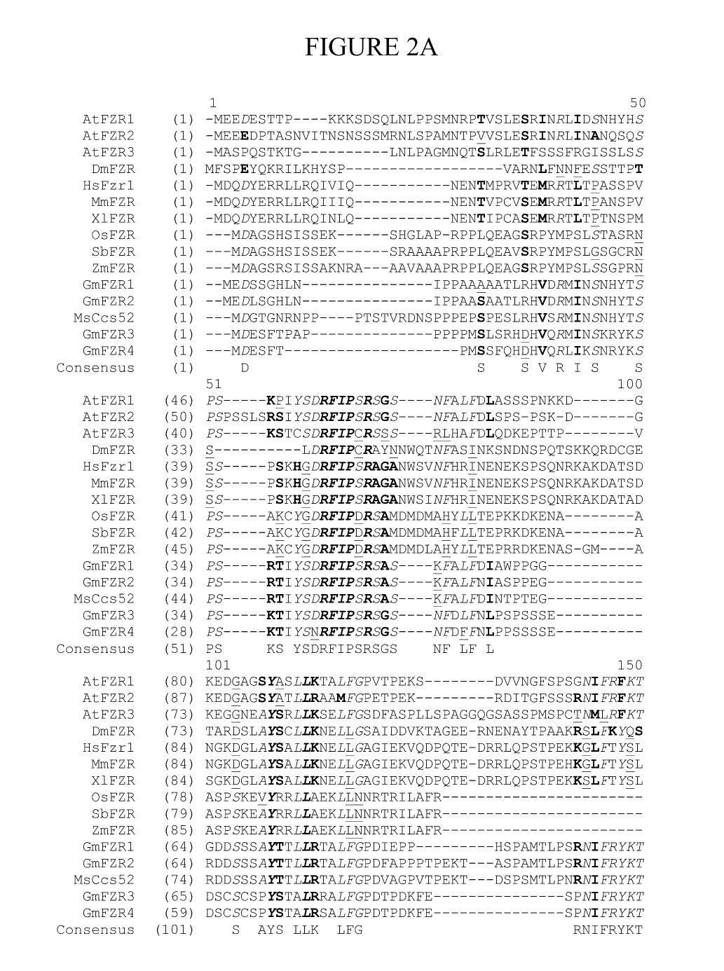

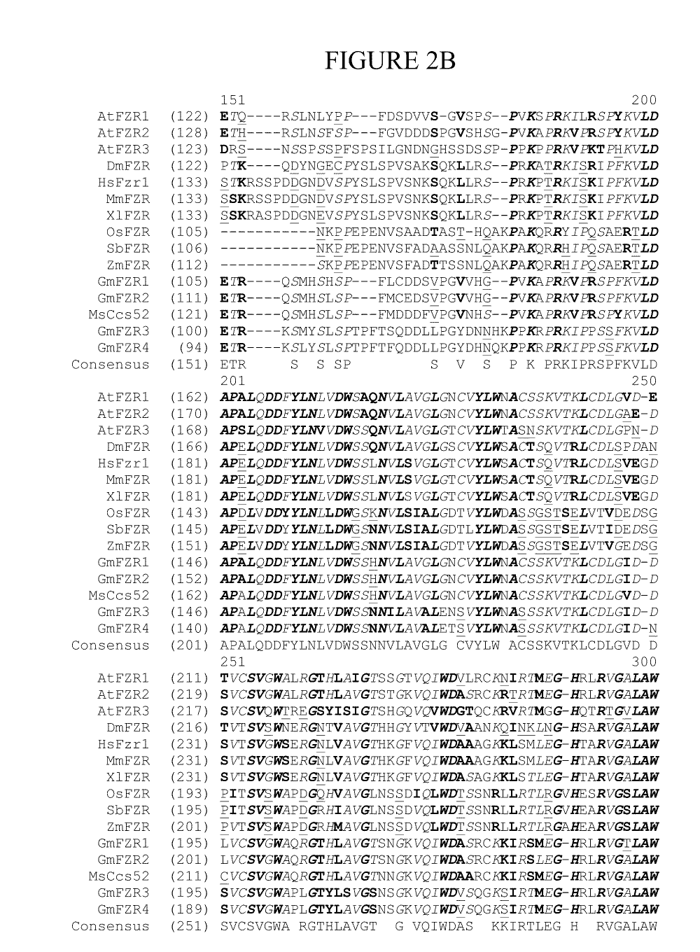

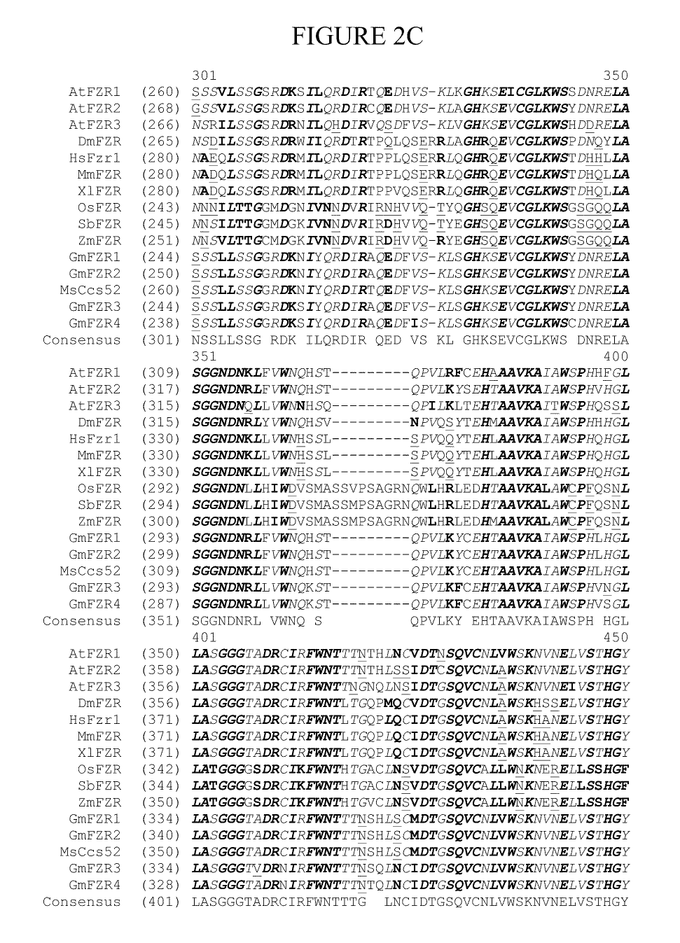

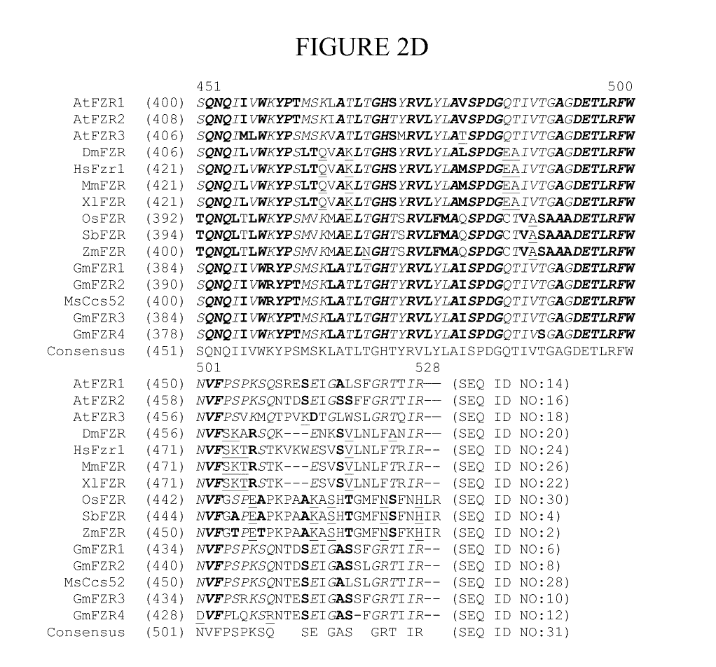

Variant amino acid sequences of ccs52 may be generated. In this example, one amino acid may be altered. Specifically, the sequences set forth in SEQ ID NO: 2, 4, 6, 8, 10 or 12 may be reviewed to determine the appropriate amino acid alteration. The selection of the amino acid to change may be made by consulting the protein alignment. See FIG. 2. An amino acid may be selected that is deemed not to be under high selection pressure (not highly conserved) and which is rather easily substituted by an amino acid with similar chemical characteristics (i.e., similar functional side-chain). Using the protein alignment set forth in FIG. 2 an appropriate amino acid can be changed. Amino acid residues that show a low percentage of sequence identity among the Zea mays ccs52 proteins are not highlighted. Additional conserved residues can be found in FIG. 2. Once the targeted amino acid is identified, a standard codon table may be used to produce variant ccs52, see, for example, Example 6A. Variants having about 70%, 75%, 80%, 81%, 82%, 83%, 84%, 85%, 86%, 87%, 88%, 89%, 90%, 91%, 92%, 93%, 94%, 95%, 96%, 97%, 98% or 99% amino acid sequence identity to SEQ ID NO: 2, 4, 6, 8, 10 or 12 may be generated using this method.

C. Additional Variant Amino Acid Sequences of ccs52

In this example, artificial protein sequences are created having 70%, 75%, 80%, 81%, 82%, 83%, 84%, 85%, 86%, 87%, 88%, 89%, 90%, 91%, 92%, 93%, 94%, 95%, 96%, 97%, 98% or 99% identity relative to the reference protein sequence. This latter effort requires identifying conserved and variable regions from the alignment set forth in FIG. 2 and then the judicious application of an amino acid substitutions table. These parts will be discussed in more detail below.

Largely, the determination of which amino acid sequences may be altered is made based on the conserved regions among ccs52 proteins. See FIG. 2. Based on the sequence alignment, the various regions of the ccs52 that can likely be altered can be determined. It is recognized that conservative substitutions can be made in the conserved regions without altering function. In addition, one of skill will understand that functional variants of the ccs52 sequences of the invention may also have minor amino acid alterations in the conserved domain.

Artificial protein sequences are then created that are different from the original in the intervals of 80-85%, 85-90%, 90-95%, and 95-100% identity. Midpoints of these intervals are targeted, with liberal latitude of plus or minus 1%, for example.

The amino acids substitutions will be effected by a custom Perl script. The substitution table is provided below in Table 2.

TABLE 2
Substitution Table

| Amino | Strongly Similar and | Rank of Order | |
|---|---|---|---|
| Acid | Optimal Substitution | to Change | Comment |
| I | L, V, M | 1 | 50:50 substitution |
| L | I, V, M | 2 | 50:50 substitution |
| V | I, L, M | 3 | 50:50 substitution |
| A | G, P, S, T | 5 | |
| G | A, P, S, T | 5 | |
| D | E, N, Q | 6 | |
| E | D, N, Q | 7 | |
| W | Y, F | 8 | |
| Y | W, F | 9 | |
| S | T, P, A, G | 10 | |
| T | S, P, A, G | 11 | |
| K | R, H | 12 | |
| R | K, H | 13 | |
| N | Q, E, D | 15 | |
| Q | N, D, E | 15 | |
| F | Y, W | 16 | |
| M | L, I, V | 17 | First methionine |
| | | | cannot change |
| H | R, K | 18 | |
| P | A, G, S, T | 19 | |
| C | | NA | No good substitutions |

First, any conserved amino acid in the protein that should not be changed is identified and “marked off” for insulation from the substitution. The start methionine will of course be added to this list automatically. Next, the changes are made. The changes will occur with isoleucine first, sweeping N-terminal to C-terminal; then leucine, and so on down the list until the desired target is reached. Interim number substitutions can be made so as not to cause reversal of changes. The list is ordered 1-19, and the first amino acid residue listed as a similar and optimal substitution is preferred, although the other listed amino acids are also suitable. See, for example, M. J. McKay et al. Sequence conservation of the rad21 Schizosaccharomyces pombe DNA double-strand break repair gene in human and mouse. Genomics. (1996) 36(2):305-15. To illustrate the substitution process, many isoleucine changes are made as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of ccs52 are generated having about 70%, 75%, 80%, 81%, 82%, 83%, 84%, 85%, 86%, 87%, 88%, 89%, 90%, 91%, 92%, 93%, 94%, 95%, 96%, 97%, 98% or 99% amino acid identity to the starting unaltered ORF sequence of SEQ ID NO: 1, 3, 5, 7, 9, or 11.

### Example 7

### Identification and Isolation of the Maize ccs52 Sequences

Total RNA was isolated from corn tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N., Anal. Biochem. 162, 156 (1987)). In brief, plant tissue samples were pulverized in liquid nitrogen before the addition of the TRIzol Reagent, and then were further homogenized with a mortar and pestle. Addition of chloroform followed by centrifugation was conducted for separation of an aqueous phase and an organic phase. The total RNA was recovered by precipitation with isopropyl alcohol from the aqueous phase.

The selection of poly(A)+RNA from total RNA was performed using PolyATract system (Promega Corporation. Madison, Wis.). In brief, biotinylated oligo(dT) primers were used to hybridize to the 3′ poly(A) tails on mRNA. The hybrids were captured using streptavidin coupled to paramagnetic particles and a magnetic separation stand. The mRNA was washed using high stringency conditions and eluted using RNAase-free deionized water.

cDNA synthesis was performed and unidirectional cDNA libraries were constructed using the SuperScript Plasmid System (Life Technology Inc. Gaithersburg, Md.). The first stand of cDNA was synthesized by priming an oligo(dT) primer containing a Not I site. The reaction was catalyzed by SuperScript Reverse Transcriptase II at 45° C. The second strand of cDNA was labeled with alpha-³²P-dCTP and a portion of the reaction was analyzed by agarose gel electrophoresis to determine cDNA sizes. cDNA molecules smaller than 500 base pairs and unligated adapters were removed by Sephacryl-5400 chromatography. The selected cDNA molecules were ligated into pSPORT1 vector in between Not I and Sal I sites. Mitotically active tissues from Zea mays were employed, including such sources as shoot cultures, immature inflorescences (tassel and ear) as well as other sources of vegetative meristems.

Individual colonies were picked and DNA was prepared either by PCR with M13 forward primers and M13 reverse primers, or by plasmid isolation. All the cDNA clones were initially sequenced using M13 reverse primers.

Functional fragments of the cell cycle protein are identified by their ability, upon introduction to cells, to stimulate the G1 to S-phase transition, which is manifested by increased DNA replication in a population of cells and by increased cell division rates.

ESTs encoding a maize ccs52 were identified by conducting BLAST (Basic Local alignment Search Toll, Altschul, S. F. et a; (1993) J Mol Biol 215:403-410; see also on the World Wide Web at www.ncbi.nlm.nih.gov/BLAST/) searches for similarity to sequence contained in the BLAST “nr” database (comprising all non-redundant GenBank CDS translations), sequences derived from the 3-dimensional structure Brookhaven Protein Data Bank, the last major release of the SWISS PROT protein sequence database, EMBL, and DDBJ databases). The cDNA sequences obtained were analyzed fro similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Cish, W and States, D. J. (1993) Nature Genetics 3:266-272 and Altschul, S. F. et al, (1997) Nucleic Acids Res. 25:3389-3402) provided by NCBI. ESTs encoding polypeptides with high homology to known ccs52 (for example, the Arabidopsis thaliana ccs52 protein, AF134835; also known as the FZR gene in various other species) were re-sequenced to obtain full-length sequence. An EST encoding the full-length ccs52 cDNA was obtained, cloned into a TOPO clone, and sequenced again to insure that the sequence was still correct. From the TOPO clone, the ccs52 gene could be moved into various expression cassettes using standard molecular biology techniques, as found in Maniatus et al (REF). The sequence of ccs52 cDNA is set forth in SEQ ID NO:1.

### Example 8

### Expression of Zmccs52 in Leaves Increases Yield

A promoter that normally drives expression of photosynthetic components, such as the maize chlorophyll a/b binding protein promoter, or CAB [Sullivan et al., 1989. Mol. Gen. Genet. 215:431-440] is used to control expression of the Zmccs52 gene. The following T-DNA is constructed in Agrobacterium strain LBA4404; RB-CAB PRO::Zmccs52::nos3′+UBI PRO::moPAT—GFP::pinII-LB. Maize immature embryos are transformed with this Agrobacterium strain and selected on 3 mg/l bialaphos to produce calli that are resistant to herbicide and exhibit green fluorescence. Plants are regenerated, and because expression of Zmccs52 is restricted to green tissues, only chlorophyll-containing cells undergo endoreduplication, leaving the roots and reproductive tissues such as the tassel and ear diploid (and thus maintaining fertility). Plants are screened for this phenotype by isolating nuclei form leaf cells and monitoring ploidy distribution within a population of nuclei. When compared to control leaves which are not expressing the transgene, samples from ccs52-expressing leaves have increased ploidy levels. Plants expressing ccs52 in plant cells having chlorophyll may produce larger ears with higher seed sets, resulting in increased yield in maize.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

What is claimed is:

1. An isolated or recombinant nucleic acid comprising a polynucleotide selected from the group consisting of:
(a) a polynucleotide that encodes the polypeptide of SEQ ID NO: 2, 4, 6, 8 or 10;
(b) a polynucleotide comprising the nucleic acid sequence set forth in SEQ ID NO: 1, 3, 5, 7, 9 or 11;
(c) a polynucleotide comprising a nucleic acid sequence of at least 85%, 90%, or 95% sequence identity to SEQ ID NO: 1, 3, 5, 7, 9 or 11, wherein the % sequence identity is based on the entire encoding region and is determined by BLAST 2.0 under default parameters wherein the polynucleotide encodes a polypeptide having cell cycle switch 52 activity;
(d) a polynucleotide encoding a polypeptide with an amino acid sequence that is at least 85%, 90%, or 95% identical to the sequence set forth in SEQ ID NO: 2, 4, 6, 8, 10 or 12, wherein the encoded polypeptide has cell cycle switch 52 activity;
(e) a polynucleotide having a nucleic acid sequence degenerate from any of (a) to (d) as a result of the genetic code; and
(f) a polynucleotide that is fully complementary to the polynucleotide of any one of (a) to (e).

2. The isolated or recombinant nucleic acid of claim 1, wherein the polynucleotide comprises a nucleic acid sequence that encodes the polypeptide of SEQ ID NO: 2, 4, 6, 8 or 10.

3. An isolated or recombinant nucleic acid according to claim 1 wherein said polynucleotide encodes a cell cycle switch 52 polypeptide that confers increased yield in a plant.

4. A recombinant DNA construct comprising at least one polynucleotide of claim 1 operably linked to a promoter.

5. A plant comprising the recombinant DNA construct of claim 4.

6. A seed comprising the recombinant DNA construct of claim 4.

7. An isolated polypeptide selected from the group consisting of a) an isolated polypeptide encoded by the nucleic acid sequence of SEQ ID NO: 1, 3, 5, 7, 9 or 11;
b) an isolated polypeptide comprising the amino acid sequence of SEQ ID NO: 2, 4, 6, 8, 10 or 12, said polypeptide having cell cycle switch 52 (ccs52) activity;
c) an isolated polypeptide comprising an amino acid sequence that is at least 85%, 90%, or 95% identical to the amino acid sequence of SEQ ID NO: 2,4, 6, 8, 10 or 12, said polypeptide having cell cycle switch 52 activity; and
d) an isolated polypeptide that is encoded by a polynucleotide comprising a nucleic acid sequence that is at least 85%, 90% or 95% identical to SEQ ID NO:
1, 3, 5, 7, 9 or 11, or a complement thereof, said polypeptide having cell cycle switch 52 activity.

8. A method of modulating the level of cell cycle switch 52 protein in a plant cell, comprising:
(a) transforming a plant cell with the recombinant DNA construct of claim 4; and
(b) regenerating a fertile transgenic plant from the plant cell of (a), wherein the fertile transgenic plant comprises the recombinant DNA construct of claim 4, and wherein the polynucleotide is expressed in a plant cell committed to becoming a differentiated plant cell having chlorophyll or a differentiated plant cell having chlorophyll, or combinations thereof, for a time sufficient to modulate the cell cycle switch 52 protein in the plant cell.

9. The method of claim 8, wherein the plant is corn, soybean, sunflower, sorghum, canola, wheat, alfalfa, cotton, rice, barley or millet.

10. The method of claim 8, wherein cell cycle switch 52 protein is increased as compared to a control plant cell, wherein the control plant cell does not contain the polynucleotide encoding the cell cycle switch 52.

11. The method of claim 8, wherein the promoter preferentially expresses the polynucleotide in a plant cell committed to becoming a differentiated plant cell having chlorophyll or a differentiated plant cell having chlorophyll, or combinations thereof, and wherein the promoter does not effectively express the polynucleotide in germline plant cells or in plant cells that will contribute to the germline of the plant.

12. The method of claim 8, wherein the expression of ccs52 in a plant cell results in increased ploidy in the plant cell as compared to a control plant cell, wherein the control plant does not contain the recombinant DNA construct.

13. The method of claim 8, wherein the expression of ccs52 in a plant results in increased yield in the plant as compared to a control plant, wherein the control plant that does not contain the recombinant DNA construct.

14. A method for increasing yield in a plant, said method comprising the steps of:
(a) introducing into plant cells the recombinant DNA construct of claim 4, wherein the promoter does not effectively express the ccs52 polynucleotide in plant germline cells but preferentially expresses the ccs52 polynucleotide in non-germline plant cells to yield transformed plant cells or combinations thereof; and
(b) regenerating a fertile transgenic plant from said transformed plant cells, wherein the fertile transgenic plant comprised the recombinant DNA construct;
wherein said ccs52 is expressed in the plant cells at levels sufficient to increase yield in said transgenic plant.

15. The method of claim 14, wherein the promoter is a promoter of a gene encoding a chlorophyll a/b binding protein.

16. The method of claim 14, wherein increased yield comprises increased ear size, increased seed set, increased chlorophyll content, increased level of photosynthetic machinery, increased cell size of said differentiated plant cells having cholorphyll, or increased overall source levels of photosynthate.

17. The method of claim 14, wherein the promoter preferentially expresses the polynucleotide in a plant cell committed to becoming a differentiated plant cell having chlorophyll or a differentiated plant cell having chlorophyll, or a combination thereof.

18. The method of claim 17, wherein the expression of ccs52 in the plant cell committed to becoming a differentiated plant cell having chlorophyll or the differentiated plant cell having chlorophyll results in increased ploidy in the plant cell as compared to a control plant cell, wherein the control plant that does not contain the polynucleotide encoding the ccs52.

19. The method of claim 18, further comprising determining the ploidy of the committed or differentiated plant cells of the plant.

20. The method of claim 19, further comprising determining duplication of the genome by isolating nuclei from leaf cells of the plant and determining ploidy of the nuclei in the cells.

21. The method of claim 14, wherein the recombinant DNA construct further comprises a second polynucleotide, wherein said second polynucleotide encodes a cell cycle GI-S transition stimulating gene, and wherein said polynucleotide is operably linked to a promoter functional in plant cells.

22. The plant of claim 5, wherein the promoter does not effectively express the ccs52 polynucleotide in plant germline cells but preferentially expresses the ccs52 polynucleotide in non-germline plant cells.

23. The plant of claim 5, wherein the promoter preferentially expresses the polynucleotide in plant cells committed to becoming differentiated plant cells having chlorophyll or differentiated plant cells having chlorophyll, or combinations thereof

24. The plant of claim 5, wherein the promoter is a promoter of a gene encoding a chlorophyll a/b binding protein.


SEQUENCE LISTING (31 sequences)

SEQ ID NO: 1 - DNA, 1434 bp, Zea mays
  CDS (1)..(1431)
atggacgcaggctcccgctcgatctcctcggcgaagaaccgcgccgccgccgtcgccgccgcgccccggccaccgctgcaggaggcgggctcccgcccctacatgccatcgctgagctcgggaccccgcaacccgtcggccaagtgctacggcgacaggttcatcccggacaggtcggcgatggacatggacttggcgcactacttgcttactgagcccaggagggacaaggagaacgcgtcgggcatggcggcgtccccgtccaaggaggcgtaccggaggctgctcgcggagaagctgctcaacaaccggacacggatcctcgctttcaggagcaagccgccggagcccgagaacgtttcttttgcggacacgacttcctccaacctgcaggccaagccggccaaacagcggcgccacattccccagtctgccgagaggaccctagacgcaccagagctagttgacgactactacctcaacttgcttgactggggaagcaacaacgtgctatccattgctttgggagacacggtgtacctgtgggatgcatcgagcggatccacatccgagcttgtgaccgtcggcgaggacagtggtcctgttacaagcgttagctgggctcctgatggtcggcacatggccgtcgggctcaactcgtctgacgtccagctctgggacaccagctccaaccgactgttgagaacactcagaggtgcgcatgaggcaagggtaggctcgctggcatggaacaacagcgtcttgaccaccggttgcatggacggcaagatcgtgaacaatgacgtaaggattagagaccacgtcgtgcagaggtacgaggggcacagccaggaggtctgcgggctcaagtggtccggatcagggcagcagctggccagcggaggcaacgacaaccttctgcacatttgggatgtgtcgatggcatcatccatgccatctgctggccgcaaccagtggctgcataggctcgaggaccacatggccgccgtgaaggcactcgcgtggtgcccgttccagagcaacttgctggccaccggcggcggtggcagcgaccgctgcatcaagttctggaacacgcacaccggtgtgtgcctgaactcggttgataccggatcacaggtgtgcgctctgctgtggaacaagaacgagagggagctgctgagctcacatggattcacacagaaccaactcaccttgtggaagtacccatcgatggtgaagatggctgaacttaatggccatacctctcgcgtcctcttcatggctcagagtcctgatggatgcacagtagcgtcggctgctgctgacgagaccctccggttctggaacgtttttggaacccctgaaacgcccaagcctgcagccaaagcttcccacactgggatgttcaacagcttcaaacatatccgatag

SEQ ID NO: 2 - protein, 477 aa, Zea mays
MDAGSRSISSAKNRAAAVAAAPRPPLQEAGSRPYMPSLSSGPRNPSAKCYGDRFIPDRSAMDMDLAHYLLTEPRRDKENASGMAASPSKEAYRRLLAEKLLNNRTRILAFRSKPPEPENVSFADTTSSNLQAKPAKQRRHIPQSAERTLDAPELVDDYYLNLLDWGSNNVLSIALGDTVYLWDASSGSTSELVTVGEDSGPVTSVSWAPDGRHMAVGLNSSDVQLWDTSSNRLLRTLRGAHEARVGSLAWNNSVLTTGCMDGKIVNNDVRIRDHVVQRYEGHSQEVCGLKWSGSGQQLASGGNDNLLHIWDVSMASSMPSAGRNQWLHRLEDHMAAVKALAWCPFQSNLLATGGGGSDRCIKFWNTHTGVCLNSVDTGSQVCALLWNKNERELLSSHGFTQNQLTLWKYPSMVKMAELNGHTSRVLFMAQSPDGCTVASAAADETLRFWNVFGTPETPKPAAKASHTGMFNSFKHIR

SEQ ID NO: 3 - DNA, 1416 bp, sorghum
  CDS (1)..(1413)
atggacgcaggatcccactcgatctcctcggagaagagccgcgccgccgccgcgccccggccgccgctgcaggaggcggtctcccgcccctacatgccatcgctgggctcgggatgccgtaacccgtcggccaagtgctacggcgacagattcatcccggacagatcggcgatggacatggacatggcacacttcctgctcactgagcccaggaaggacaaggagaacgcggcggcgtccccgtccaaggaggcgtaccggaggctgctcgcggagaagctgctcaacaaccggacacggatcctcgccttcaggaacaagccgccggagcccgagaacgtatctttcgccgatgcggcttcctccaacctgcaggccaagcctgctaagcagcggcgccacattccccagtctgccgagaggaccctagacgccccagagcttgttgatgactactacctcaacctgcttgactgggggagcaacaatgtgctgtccattgctctgggagacacactgtacctgtgggatgcgtcgagtggatccacatccgagcttgtgaccatcgatgaggacagcggtcctattaccagtgttagctgggctcctgatggtcggcacatcgccgtggggctcaactcgtccgacgtccagctttgggacaccagctccaaccgactgttgagaacactcagaggtgtgcatgaggcaagggtaggttcactggcatggaacaacagcatcctaaccaccggtggcatggatggcaagattgtgaacaatgacgtgaggattagagaccacgttgtgcagacttacgaggggcacagccaggaggtgtgcgggctcaagtggtctggatcagggcagcagctggccagcggaggcaacgacaaccttctgcacatttgggatgtgtcgatggcatcatccatgccatctgctggccgcaaccagtggctacataggctcgaggaccacacggccgccgtgaaggcactcgcgtggtgcccgttccagagcaacttgcttgccactggcggtggtggcagcgatcgttgcatcaagttctggaacacacacactggtgcgtgcctgaactcagttgacaccggatcacaggtgtgcgctcttctctggaacaagaatgagagggagctgctgagttcacatggattcacacagaaccaactgactttgtggaagtacccatcgatggtgaagatggctgaacttactggccatacctctcgtgtccttttcatggctcagagtcctgatggatgcacagtagcgtcagctgctgcagatgagaccctccggttctggaacgtttttggagcccctgaagcgcccaagcctgctgccaaagcttcccacactgggatgttcaacagcttcaaccatatccgatag

SEQ ID NO: 4 - protein, 471 aa, sorghum
MDAGSHSISSEKSRAAAAPRPPLQEAVSRPYMPSLGSGCRNPSAKCYGDRFIPDRSAMDMDMAHFLLTEPRKDKENAAASPSKEAYRRLLAEKLLNNRTRILAFRNKPPEPENVSFADAASSNLQAKPAKQRRHIPQSAERTLDAPELVDDYYLNLLDWGSNNVLSIALGDTLYLWDASSGSTSELVTIDEDSGPITSVSWAPDGRHIAVGLNSSDVQLWDTSSNRLLRTLRGVHEARVGSLAWNNSILTTGGMDGKIVNNDVRIRDHVVQTYEGHSQEVCGLKWSGSGQQLASGGNDNLLHIWDVSMASSMPSAGRNQWLHRLEDHTAAVKALAWCPFQSNLLATGGGGSDRCIKFWNTHTGACLNSVDTGSQVCALLWNKNERELLSSHGFTQNQLTLWKYPSMVKMAELTGHTSRVLFMAQSPDGCTVASAAADETLRFWNVFGAPEAPKPAAKASHTGMFNSFNHIR

SEQ ID NO: 5 - DNA, 1380 bp, Glycine max
  CDS (1)..(1377)
atggaggactcgtccggccacctgaatattcctccggccgccgcggcggcgactctccggcacgttgaccgcatgatcaactccaaccactacacctcgccttccagaacaatctactccgaccgcttcattcccagcagatctgcctcgaaattcgcgctcttcgacatcgcctggcctcccgggggcggcgacgacagctccagcgcctacaccacgctcctccgcaccgcgctcttcggccccgacatcgagccgccgcactcgccggcgatgactctccccagccggaatatcttccgttacaaaaccgagacgcgccagtccatgcactcgcactcgccgttcttgtgcgacgattcggtccccggcgttgtccacggcccggtcaaggctccgaggaaggttccgaggtcgccttttaaggttctggatgcgcctgcgctgcaagatgatttttacctgaatcttgtggattggtcttcgcataatgtgttggctgttggtctgggaaactgtgtttatctctggaatgcttgtagcagcaaggttactaaattatgtgacttggggattgatgacctcgtttgttcggttggctgggctcagcgtggtacacaccttgctgttggaactagcaatggtaaagttcagatttgggatgcatctcgatgcaagaagataagatctatggagggtcatcggttacgggttgggaccttggcttggagttcatctcttttgtcttctggcggcagggataagaatatttatcaaagagatatccgtgcacaagaagattttgtcagtaaattgtcagggcacaaatcagaggtttgtggactgaagtggtcttatgataaccgtgagttggcatctggaggaaatgacaacagattgtttgtttggaatcaacactcaactcagcctgtcctgaagtactgtgagcatacagcagctgttaaagctattgcatggtctcctcatcttcacggacttcttgcatctgggggaggaactgcagaccgatgcatacgtttctggaatacaaccacaaactcacatttaagctgcatggacacgggaagtcaggtttgcaatcttgtctggtccaaaaatgtcaatgaactagtaagcacgcatggctattcccagaaccagataattgtttggagatacccctccatgtcaaagttggccactcttacgggtcatacctacagagttctttatcttgccatttctccggatggacagactattgtaactggagctggtgatgaaacacttaggttctggaacgtattcccttcccctaaatcacagaatactgatagtgaaatcggagcatcatcttttggaagaacaattattaggtga

SEQ ID NO: 6 - protein, 459 aa, Glycine max
MEDSSGHLNIPPAAAAATLRHVDRMINSNHYTSPSRTIYSDRFIPSRSASKFALFDIAWPPGGGDDSSSAYTTLLRTALFGPDIEPPHSPAMTLPSRNIFRYKTETRQSMHSHSPFLCDDSVPGVVHGPVKAPRKVPRSPFKVLDAPALQDDFYLNLVDWSSHNVLAVGLGNCVYLWNACSSKVTKLCDLGIDDLVCSVGWAQRGTHLAVGTSNGKVQIWDASRCKKIRSMEGHRLRVGTLAWSSSLLSSGGRDKNIYQRDIRAQEDFVSKLSGHKSEVCGLKWSYDNRELASGGNDNRLFVWNQHSTQPVLKYCEHTAAVKAIAWSPHLHGLLASGGGTADRCIRFWNTTTNSHLSCMDTGSQVCNLVWSKNVNELVSTHGYSQNQIIVWRYPSMSKLATLTGHTYRVLYLAISPDGQTIVTGAGDETLRFWNVFPSPKSQNTDSEIGASSFGRTIIR

SEQ ID NO: 7 - DNA, 1398 bp, Glycine max
  CDS (1)..(1395)
atggaggacttgtccggccacctgaatattcctccggccgcctccgcggcgactctccgccacgtggaccgcatgatcaactccaaccactacacctcgccttccaggacaatctactccgaccgcttcattcccagcagatctgcctcgaaattcgcactcttcaacatcgcttcgccgcccgagggccgcgacgacagctccagtgcctacaccacgctcctccgcaccgcgctcttcggccccgacttcgcgccgccgcccacgccggagaaaacggcctcgccggcgatgacgctccccagccgaaatattttccggtacaagacagagacgcgccagtccatgcactcgctctcgccattcatgtgcgaggattcggtgcccggcgttgttcacggtccggtcaaggctccgaggaaggttccgaggtcgccttttaaggttctggatgcgcctgcgctgcaagacgatttctacctgaatcttgtggattggtcttcgcataatgtgttggctgttggtctgggaaactgtgtttatctttggaatgcttgtagcagcaaggttactaaattatgtgacttggggattgatgaccttgtttgttcggttggctgggctcagcgtggtacacaccttgctgttggaacaagcaatggtaaagttcagatttgggatgcatctcgatgcaagaagataagatctctggagggtcatcggttacgtgttggggccttggcttggagttcatctcttttgtcttctggtggcagggataagaatatttatcaaagagatatccgtgcacaagaagattttgtcagtaaattatcagggcacaaatcagaggtttgtggactgaagtggtcttatgataaccgtgagttggcatctggaggaaatgacaacagattgtttgtttggaatcaacactcaactcagcctgtcctaaagtactgtgagcatacagcagctgttaaagctattgcatggtctcctcatcttcatggacttcttgcatctgggggaggaactgcagaccgatgcatacgtttctggaatacaaccacaaactcacacttaagctgcatggacactggaagccaggtttgcaatcttgtctggtccaaaaatgtcaatgaactagtaagtacacatggctattcccagaatcagataattgtttggagataccccaccatgtcaaagttggccactcttacaggccatacctatagagttctttatctagccatttctcccgatggacagactattgtaactggagctggagatgaaacacttaggttctggaacgtattcccttcccctaaatcacagaatactgatagtgaaatcggagcatcatctcttggaagaacaattattaggtga

SEQ ID NO: 8 - protein, 465 aa, Glycine max
MEDLSGHLNIPPAASAATLRHVDRMINSNHYTSPSRTIYSDRFIPSRSASKFALFNIASPPEGRDDSSSAYTTLLRTALFGPDFAPPPTPEKTASPAMTLPSRNIFRYKTETRQSMHSLSPFMCEDSVPGVVHGPVKAPRKVPRSPFKVLDAPALQDDFYLNLVDWSSHNVLAVGLGNCVYLWNACSSKVTKLCDLGIDDLVCSVGWAQRGTHLAVGTSNGKVQIWDASRCKKIRSLEGHRLRVGALAWSSSLLSSGGRDKNIYQRDIRAQEDFVSKLSGHKSEVCGLKWSYDNRELASGGNDNRLFVWNQHSTQPVLKYCEHTAAVKAIAWSPHLHGLLASGGGTADRCIRFWNTTTNSHLSCMDTGSQVCNLVWSKNVNELVSTHGYSQNQIIVWRYPTMSKLATLTGHTYRVLYLAISPDGQTIVTGAGDETLRFWNVFPSPKSQNTDSEIGASSLGRTIIR

SEQ ID NO: 9 - DNA, 1380 bp, Glycine max
  CDS (1)..(1377)
atggacgaatcattcactccagcaccaccacctcctccaatgtccctttctcggcacgatcacgtccaacgaatgataaactcgaagcgctacaagtcaccttcgaaaacaatatactccgacaggttcattccgagcagatccggttccaatttcgatctcttcaatctaccttcgccgtcgtcgtcagaggacagttgcagttgcagcccctacagcaccgcgctgcggagggccttgttcggaccagacactcccgataaatttgaaagccctaatatattccgttacaaaacggagactcgaaagtctatgtattctctctcacccaccccttttacttcccaggatgatcttctccctggttatgacaacaatcataaacctcccaagcgtcctcgcaagattcctccctcctcttttaaggttttggacgcccctgcgctgcaagacgatttttatctgaatctcgtggattggtcatccaacaatatcttggctgtggctctggagaactctgtttatttgtggaatgcttctagcagcaaggtaactaaattatgcgatttggggattgacgattcagtttgttcagttggctgggctccacttggtacctacctgtctgttggatcaaacagtggtaaagtccagatttgggatgtatctcaaggcaagtcaataagaactatggagggtcatcgtttacgtgttggggccttggcttggagttcctctcttttgtcttctggtggccgggataaaagcatttatcaacgagatatacgtgcacaggaggattttgtcagtaaactgtctgggcacaagtcagaggtttgtggactgaagtggtcttatgacaaccgtgagctagcatctggaggaaatgacaacaggttgcttgtttggaatcaaaagtcaacccagcccgttctgaagttctgtgagcatacagcagctgttaaagctattgcatggtctcctcatgtaaatggacttcttgcatctggaggaggaactgtggaccgaaacattcgcttttggaatacaaccacaaactcacagttaaactgtatcgacactggtagtcaggtttgtaaccttgtttggtctaaaaatgtgaatgaactcgtaagcacacatggttactcccagaaccagataatagtttggaaatatcccaccatgtcaaagctagcaacgcttacaggccatacttacagagttctttatcttgccatatctcctgacgggcagactatcgtcactggagctggagatgaaactcttaggttctggaatgtattcccttcgcggaaatcacagaatactgagagtgaaattggagcttcatcttttggcagaactatcatcagatga

SEQ ID NO: 10 - protein, 459 aa, Glycine max
MDESFTPAPPPPPMSLSRHDHVQRMINSKRYKSPSKTIYSDRFIPSRSGSNFDLFNLPSPSSSEDSCSCSPYSTALRRALFGPDTPDKFESPNIFRYKTETRKSMYSLSPTPFTSQDDLLPGYDNNHKPPKRPRKIPPSSFKVLDAPALQDDFYLNLVDWSSNNILAVALENSVYLWNASSSKVTKLCDLGIDDSVCSVGWAPLGTYLSVGSNSGKVQIWDVSQGKSIRTMEGHRLRVGALAWSSSLLSSGGRDKSIYQRDIRAQEDFVSKLSGHKSEVCGLKWSYDNRELASGGNDNRLLVWNQKSTQPVLKFCEHTAAVKAIAWSPHVNGLLASGGGTVDRNIRFWNTTTNSQLNCIDTGSQVCNLVWSKNVNELVSTHGYSQNQIIVWKYPTMSKLATLTGHTYRVLYLAISPDGQTIVTGAGDETLRFWNVFPSRKSQNTESEIGASSFGRTIIR

SEQ ID NO: 11 - DNA, 1359 bp, Glycine max
  CDS (1)..(1356)
atggacgaatcattcactccaatgtcgtcttttcaacatgaccacgtccaacgattgataaagtcgaaccgctacaagtcaccttccaaaacaatctactccaacaggttcattcctagcagatccggttccaattttgatttcttcaatctacctccgtcgtcctcgtcagaggacagttgcagttgcagtccctacagcaccgcgctgcgaagtgccttgttcggaccagacactcccgataaatttgaaagccctaatatattccgttacaaaacggagactcgaaagtccttgtattctctctcacccaccccctttactttccaggatgaccttctccctggttatgaccacaatcaaaaacctcctaagcgtcctcgcaagattcctccttcgtcttttaaggttttggacgcccctgccctgcaagacgatttttatctgaatctcgtggattggtcctccaacaatgtcttggctgtggctctggagacctctgtttatttgtggaatgcttctagcagcaaggtaactaaattatgtgatttggggattgacaactcagtttgttcggttggctgggctccacttggtacctacctggctgttggatcaaacagtggtaaagtccagatttgggatgtatctcaaggcaagtcaataagaactatggagggtcatcgtttacgtgttggagctttggcctggagttcttctcttttgtcttctggtggccgggataaaagtatttatcaacgagatattcgtgcacaggaggatttcatcagtaaactgtctggacacaagtcagaggtctgtggactgaagtggtcttgtgataaccgtgagctagcatccggaggaaatgacaacaggttgcttgtttggaatcaaaagtcaacccagcccgtcctgaagttctgtgagcatacagcagctgttaaagctattgcgtggtctcctcatgtaagtggacttcttgcatctggaggaggaactgcggaccgaaacattcgattttggaatacaaccacaaacacacagttaaactgtatcgacactggtagtcaggtttgtaaccttgtttggtctaaaaatgtgaatgaacttgtaagcacacatggttactcccagaaccagataatagtttggaaataccccaccatgtcaaagctagcaactcttacaggccatacttacagagttctttatcttgccatatctcctgatggacagactatcgtcagtggggctggagacgaaactcttaggttttgggatgtattccctttgcagaaatcacggaataccgagagtgaaattggtgcatcttttgggagaactatcattagatga

SEQ ID NO: 12 - protein, 452 aa, Glycine max
MDESFTPMSSFQHDHVQRLIKSNRYKSPSKTIYSNRFIPSRSGSNFDFFNLPPSSSSEDSCSCSPYSTALRSALFGPDTPDKFESPNIFRYKTETRKSLYSLSPTPFTFQDDLLPGYDHNQKPPKRPRKIPPSSFKVLDAPALQDDFYLNLVDWSSNNVLAVALETSVYLWNASSSKVTKLCDLGIDNSVCSVGWAPLGTYLAVGSNSGKVQIWDVSQGKSIRTMEGHRLRVGALAWSSSLLSSGGRDKSIYQRDIRAQEDFISKLSGHKSEVCGLKWSCDNRELASGGNDNRLLVWNQKSTQPVLKFCEHTAAVKAIAWSPHVSGLLASGGGTADRNIRFWNTTTNTQLNCIDTGSQVCNLVWSKNVNELVSTHGYSQNQIIVWKYPTMSKLATLTGHTYRVLYLAISPDGQTIVSGAGDETLRFWDVFPLQKSRNTESEIGASFGRTIIR

SEQ ID NO: 13 - DNA, 1428 bp, Arabidopsis thaliana
  CDS (1)..(1425)
atggaagaagatgaatcaacaacaccgaagaagaagtcagattctcagctgaatcttccaccgtcgatgaatcgtccgacggtgtcactcgagtcacgaatcaaccgtttaatcgattcgaatcattatcactctccttcgaaacctatttactcagataggtttattccaagtagatctggttccaatttcgcgcttttcgatttggcatcttcttcgccgaataagaaagatgggaaagaagatggggctggttcttatgcgagtcttttgaaaacggcgctttttggtccggtgacgccggagaaaagtgatgttgttaatgggttttctccgtcggggaatatttttaggtttaagacggaaacgcagaggtctttgaatttgtatccgccttttgattctgatgtggttagtggtgttagccctagtcctgttaagtcgccgaggaagattcttaggtctccttataaggtattggatgcgccggctctgcaagatgatttttacttgaatctcgtggattggtcggcacaaaatgttcttgcagtgggattagggaactgcgtgtatttgtggaatgcttgtagtagcaaggtaactaagttatgcgatcttggggtcgatgaaactgtttgctcagttggttgggcactacgtggaacacatttggctattggaactagtagcggaacagtacagatatgggatgtgttacgatgcaagaatattagaacaatggaaggtcatcggctaagagttggagccttggcatggagctcatctgttctgtcttcaggtagtagagacaagagcatacttcagagagacattcgtactcaagaagatcatgtcagtaaacttaaaggtcacaaatccgaaatctgtggactcaaatggtcttcggacaatcgcgagctagcatcaggtggaaacgacaataagctgtttgtatggaaccaacattcaacacaaccggttttgagattctgtgaacacgccgcagcagtgaaagccattgcgtggtctccacatcattttggacttcttgcttctggtggtggcactgctgatagatgtatccgtttctggaacacaacgacaaacactcatttaaattgcgtagataccaacagccaggtgtgtaatttggtatggtctaagaacgtgaatgagcttgtgagcacacacgggtattcccagaaccaaatcatcgtctggaaatatccaaccatgtctaaattggcaactctcactggtcactcgtaccgcgttctgtaccttgcggtgtcaccggatggacagacaattgtgacaggagcaggagatgaaaccttgagattctggaatgtcttcccttctccgaaatctcagagcagggagagcgaaatcggggcattatcttttggtagaacaacaatccggtga

SEQ ID NO: 14 - protein, 475 aa, Arabidopsis thaliana
MEEDESTTPKKKSDSQLNLPPSMNRPTVSLESRINRLIDSNHYHSPSKPIYSDRFIPSRSGSNFALFDLASSSPNKKDGKEDGAGSYASLLKTALFGPVTPEKSDVVNGFSPSGNIFRFKTETQRSLNLYPPFDSDVVSGVSPSPVKSPRKILRSPYKVLDAPALQDDFYLNLVDWSAQNVLAVGLGNCVYLWNACSSKVTKLCDLGVDETVCSVGWALRGTHLAIGTSSGTVQIWDVLRCKNIRTMEGHRLRVGALAWSSSVLSSGSRDKSILQRDIRTQEDHVSKLKGHKSEICGLKWSSDNRELASGGNDNKLFVWNQHSTQPVLRFCEHAAAVKAIAWSPHHFGLLASGGGTADRCIRFWNTTTNTHLNCVDTNSQVCNLVWSKNVNELVSTHGYSQNQIIVWKYPTMSKLATLTGHSYRVLYLAVSPDGQTIVTGAGDETLRFWNVFPSPKSQSRESEIGALSFGRTTIR

SEQ ID NO: 15 - DNA, 1452 bp, Arabidopsis thaliana
  CDS (1)..(1449)
atggaagaagaagatcctacagcaagcaatgtgataacgaattcgaattcttcatctatgagaaacctatcgccggcgatgaatactccggtggtttcacttgagtcacgaatcaatcgattaatcaatgctaatcaatctcaatcaccatcaccatcatcactatcaaggtctatatactctgatagatttatccccagtagatccggatccaatttcgctcttttcgatctatctccttctcctagtaaagatggtaaggaagatggagctggctcttacgctactctgttgcgtgcggcgatgtttggtcctgagacgccggagaagagagatattactgggttttcttcttccaggaatatttttaggtttaagacggagactcatcggtctttgaattcgttttctccttttggtgttgatgatgattctcctggtgtttctcatagtggtcctgttaaagctcccaggaaagtgccgcgatcgccgtataaggtattggatgcaccggccttgcaagatgatttttatctgaatcttgtggattggtctgcacaaaatgttctagcagtgggactagggaactgtgtgtatttatggaatgcttgtagcagcaaggttactaagttatgtgatctcggagctgaggatagtgtttgctcagtgggttgggcgttacgtggaactcatctggctgttggaactagtaccgggaaagttcagatatgggatgcgtcacgctgcaagagaacaagaacaatggaaggtcatcgtctaagagttggagccctggcatggggttcatcggttctgtcatctggtagcagagacaagagtattcttcagagagacataaggtgtcaagaagatcatgtcagtaaattggcaggtcataaatctgaggtatgcggactcaagtggtcttatgacaacagagagctagcatctggtggaaacgacaataggctttttgtatggaaccaacattcaacacaaccggttttgaaatatagtgaacacactgcagctgttaaagccattgcttggtctcctcatgttcatgggcttcttgcttctggtggtggtactgctgatagatgcatacgtttttggaatacaaccacgaatactcatttaagttccatagatacttgcagtcaggtatgcaatctagcttggtctaagaacgtaaacgagcttgttagcacacacggatactctcagaaccaaatcattgtctggaaatacccaaccatgtccaaaattgctactctaaccggtcacacataccgagtcttataccttgcggtttcacccgatggacagacgattgtaacaggagcaggagatgaaaccttaaggttctggaatgttttcccttccccaaaatctcagaacacggatagtgaaatcgggtcgtctttctttggtagaacaacaattcggtga

SEQ ID NO: 16 - protein, 483 aa, Arabidopsis thaliana
MEEEDPTASNVITNSNSSSMRNLSPAMNTPVVSLESRINRLINANQSQSPSPSSLSRSIYSDRFIPSRSGSNFALFDLSPSPSKDGKEDGAGSYATLLRAAMFGPETPEKRDITGFSSSRNIFRFKTETHRSLNSFSPFGVDDDSPGVSHSGPVKAPRKVPRSPYKVLDAPALQDDFYLNLVDWSAQNVLAVGLGNCVYLWNACSSKVTKLCDLGAEDSVCSVGWALRGTHLAVGTSTGKVQIWDASRCKRTRTMEGHRLRVGALAWGSSVLSSGSRDKSILQRDIRCQEDHVSKLAGHKSEVCGLKWSYDNRELASGGNDNRLFVWNQHSTQPVLKYSEHTAAVKAIAWSPHVHGLLASGGGTADRCIRFWNTTTNTHLSSIDTCSQVCNLAWSKNVNELVSTHGYSQNQIIVWKYPTMSKIATLTGHTYRVLYLAVSPDGQTIVTGAGDETLRFWNVFPSPKSQNTDSEIGSSFFGRTTIR

SEQ ID NO: 17 - DNA, 1446 bp, Arabidopsis thaliana
  CDS (1)..(1443)
atggcatcgccacagagtaccaaaaccggtttgaatcttcccgcagggatgaaccagacttcgttgcgtttagaaacgttttcgagctcatttcgtgggatttcgagtttatcctcgccgtctaaatcgacttgcagcgacagattcataccgtgcagatcttcttcgagactccacgctttcgatctgcaggataaggaaccgactaccccggttaaagagggagggaatgaagcctattctagacttttgaaatctgagcttttcggatctgattttgcttctcctcttttgtctcctgcaggtggtcaaggatctgcttcttctccgatgagtccatgtactaacatgttgagattcaagacggatcgttccaattcaagtcctagttcgccgttttctccttccattctcggaaatgataatggtcactccagtgactcgtctccgcctccgaagccccctcgcaaggttcctaaaacacctcataaggtcttggatgctccttctttacaagatgacttctacttgaatgttgtggactggagttcacagaatgttcttgccgttgggcttggtacttgtgtttatctttggacggcttccaatagcaaagtgactaagttatgtgacctagggcctaatgacagtgtgtgctcggttcagtggacacgggaaggttcatatatatctattggtacaagtcatggtcaggttcaggtttgggacggaacacagtgcaagagagtccgaaccatgggaggtcatcaaacaagaactggtgtcttggcatggaactcaaggatcttatcatcagggagcagagacagaaacatccttcagcatgatatccgagtccagagcgattttgtcagcaaactcgtgggccacaaatctgaagtctgcggtctgaaatggtctcatgatgatagagagcttgcgtctggaggcaatgataatcagttattggtatggaacaatcattcacagcaacctattctgaagctgactgagcatacagcagcggttaaggcaattacatggtctcctcatcagagcagcctccttgcttcgggaggtggaaccgcagacagatgcattagattctggaacacgacaaacggaaatcagttaaacagcatcgacactgggagccaggtctgcaatctggcgtggagcaagaatgttaacgagatagtgagtactcatggatactctcaaaaccaaatcatgctctggaagtacccatccatgtcaaaggttgcaacacttacgggccacagtatgagagtactttacttggctacgtcacctgatggccagactatagtgaccggagcaggagatgagaccctgcggttttggaacgtctttccttcagtaaaaatgcagacaccagtgaaagacacaggtctctggtcattggggaggacacagatccgataa

SEQ ID NO: 18 - protein, 481 aa, Arabidopsis thaliana
MASPQSTKTGLNLPAGMNQTSLRLETFSSSFRGISSLSSPSKSTCSDRFIPCRSSSRLHAFDLQDKEPTTPVKEGGNEAYSRLLKSELFGSDFASPLLSPAGGQGSASSPMSPCTNMLRFKTDRSNSSPSSPFSPSILGNDNGHSSDSSPPPKPPRKVPKTPHKVLDAPSLQDDFYLNVVDWSSQNVLAVGLGTCVYLWTASNSKVTKLCDLGPNDSVCSVQWTREGSYISIGTSHGQVQVWDGTQCKRVRTMGGHQTRTGVLAWNSRILSSGSRDRNILQHDIRVQSDFVSKLVGHKSEVCGLKWSHDDRELASGGNDNQLLVWNNHSQQPILKLTEHTAAVKAITWSPHQSSLLASGGGTADRCIRFWNTTNGNQLNSIDTGSQVCNLAWSKNVNEIVSTHGYSQNQIMLWKYPSMSKVATLTGHSMRVLYLATSPDGQTIVTGAGDETLRFWNVFPSVKMQTPVKDTGLWSLGRTQIR

SEQ ID NO: 19 - DNA, 1581 bp, Drosophila melanogaster
  CDS (1)..(1578)
atgtcgcagttcaattttgtgagcgatttgcagaatgctctcatcatggacggcgagacgcgcggacctgcgcccaggtggaagaagaagctggaggcgtctctaaatggaagtgtgaataccactcggtcggtgctatccgtctcgtacaacaccagtttctcgggtgtccaggcgcccacgaaaactccgggcaagagcagcgagggcaagaccaagaagtccaacaccacgccctctaagacgccaggaggcggagatcgctttattccgaatcgggcggctaccaactttgagttagcacactttctggtgaacaaagactccggcgataagtccgatgaggagaacgacaaggccacctcgagcaacagcaacgagagcaatgtccaggcttcggctcacaagggcgaccggcagaaactcatctctgaagtggcccaggtcggtgactccaagggcgggcgcattttgtgctaccaaaacaaggctcccgctgctccagaaacacacaacaatcccctgaaggtcgtgtactccattaagacacccatatccacaaagagtggctcacgctatatacccaccacatccgagaggattctggatgcacctgattttattaacgattactatttaaatcttatggattggagtgccgacaatatagtggctgtggccttgggcagttgcgtctatttgtggaacgcacagaccggaaatatcgagcagcttacggagtttgaggagggcgactacgcaggctcgctatcgtggatccaggaggggcagatacttgccatcggcaacagcaccggtgccgtggagctgtgggactgctccaaagtgaagcgtctgcgagtgatggatggacacagtgcccgagtgggatccttggcctggaactcattcctggtttcctctggcagccgggatggcaccattgtccaccacgatgtgcgtgcacgtgagcacaagctttccacattgtccggacacacgcaggaggtttgcggcctaaagtggtccacggatttcaagtatttggctagcggaggcaacgacaatctggtgaatgtttggtcggcggccagcggtggcgtgggaactgccaccgatcccttgcacaaattcaacgaccatcaagctgcagtgcgtgccttggcctggtgtccctggcaaccaagtactctagcctctggaggcggcaccgccgatcgctgcatcaagttctggaatgtgaacaatggcactttaatgaaatccgtggactccaagtcgcaggtctgttctctgctcttttctcgccactacaaggagctgatctctgcgcatggttttgctaacaaccaactgaccatttggaaatacccaacaatggtgaagcaagccgatttgactggacacacgtcacgagttctccagatggccatgtctccggacggcagcacagtgatcagcgccggagctgatgaaaccctgcgtctttggaactgcttcgctcccgatccgttggcgtccaagaaggcagtttcgaccagcaagggcaaacagagcgtgttccgacagagcatccgttga

SEQ ID NO: 20 - protein, 526 aa, Drosophila melanogaster
MSQFNFVSDLQNALIMDGETRGPAPRWKKKLEASLNGSVNTTRSVLSVSYNTSFSGVQAPTKTPGKSSEGKTKKSNTTPSKTPGGGDRFIPNRAATNFELAHFLVNKDSGDKSDEENDKATSSNSNESNVQASAHKGDRQKLISEVAQVGDSKGGRILCYQNKAPAAPETHNNPLKVVYSIKTPISTKSGSRYIPTTSERILDAPDFINDYYLNLMDWSADNIVAVALGSCVYLWNAQTGNIEQLTEFEEGDYAGSLSWIQEGQILAIGNSTGAVELWDCSKVKRLRVMDGHSARVGSLAWNSFLVSSGSRDGTIVHHDVRAREHKLSTLSGHTQEVCGLKWSTDFKYLASGGNDNLVNVWSAASGGVGTATDPLHKFNDHQAAVRALAWCPWQPSTLASGGGTADRCIKFWNVNNGTLMKSVDSKSQVCSLLFSRHYKELISAHGFANNQLTIWKYPTMVKQADLTGHTSRVLQMAMSPDGSTVISAGADETLRLWNCFAPDPLASKKAVSTSKGKQSVFRQSIR

SEQ ID NO: 21 - DNA, 1482 bp, Xenopus laevis
  CDS (1)..(1479)
atggaccaggattatgagagacggcttctacggcagatcaacctccagaatgagaacaccataccttgtgcatcagaaatgaggaggacattaacaccaacaaactctcccatgtcttcaccaagtaaacatggagacaggtttataccatcgcgggcaggagcgaattggagcattaacttccacagaataaatgagaatgaaaaatcccccagtcagaacagaaaagccaaggatgccacggcagacagtggcaaagatggcctagcttattcagctctgcttaagaatgagctgttgggagctggtattgagaaagttcaagatccacagactgaagatagacgactacaaccatcaacgccagaaaaaaagagcctgtttacgtactctctaagttctaagcgtgcaagcccagatgatggaaatgaagtgtctccttattctttgtcacctgtcagtaataaaagccagaagctattgcggtctccacgaaagcctacaaggaagatttcgaagatcccctttaaagttctggatgcacctgagttacaagatgacttttacctcaacctagtggattggtcatccctcaatgttttaagtgtgggcttgggaacttgtgtgtacttatggagtgcatgtactagccaggtgactagactatgtgacctctctgtggaaggggattctgtaacttcagttggttggtcagagcggggaaacctggtagctgtggggacacacaaaggctttgttcagatttgggatgcatcagcagggaagaaactgtccactctggagggacacacagcccgtgtgggagccttggcttggaatgcagaccagctttcctctgggagtagggacaggatgatcttacaaagggatattcgcacaccacctgttcagtcagagagaaggctgcaaggccacaggcaagaagtgtgtggcctcaagtggtccacagaccaccagctcctggcttcaggagggaatgacaacaagctcttggtttggaaccattctagcctgagtcctgttcaacagtatactgaacatctagctgcagtgaaggcaattgcctggtcccctcatcaacatggccttttggcttcaggcgggggcactgctgaccgctgcatccgcttctggaacacactgacaggccagcctttacagtgtattgatactggctctcaagtctgtaacttggcctggtctaagcatgcaaatgaactggtaagcacacatggctactctcaaaatcagatattagtatggaaatatccatcgcttactcaggtggccaagttgactggacattcctaccgtgttctctacctggctatgtcaccagatggggaggccattgttacaggagcaggtgatgaaacgctgagattctggaatgtttttagcaaaacacgctccacaaaggagtctgtctcggttctaaacctgttcacgagaatacgatag

SEQ ID NO: 22 - protein, 493 aa, Xenopus laevis
MDQDYERRLLRQINLQNENTIPCASEMRRTLTPTNSPMSSPSKHGDRFIPSRAGANWSINFHRINENEKSPSQNRKAKDATADSGKDGLAYSALLKNELLGAGIEKVQDPQTEDRRLQPSTPEKKSLFTYSLSSKRASPDDGNEVSPYSLSPVSNKSQKLLRSPRKPTRKISKIPFKVLDAPELQDDFYLNLVDWSSLNVLSVGLGTCVYLWSACTSQVTRLCDLSVEGDSVTSVGWSERGNLVAVGTHKGFVQIWDASAGKKLSTLEGHTARVGALAWNADQLSSGSRDRMILQRDIRTPPVQSERRLQGHRQEVCGLKWSTDHQLLASGGNDNKLLVWNHSSLSPVQQYTEHLAAVKAIAWSPHQHGLLASGGGTADRCIRFWNTLTGQPLQCIDTGSQVCNLAWSKHANELVSTHGYSQNQILVWKYPSLTQVAKLTGHSYRVLYLAMSPDGEAIVTGAGDETLRFWNVFSKTRSTKESVSVLNLFTRIR

SEQ ID NO: 23 - DNA, 1491 bp, Homo sapiens
  CDS (1)..(1488)
atggaccaggactatgagcggcgcctgcttcgccagatcgtcatccagaatgagaacacgatgccacgcgtcacagagatgcggcggaccctgacgcctgccagctccccagtgtcctcgcccagcaagcacggagaccgcttcatcccctccagagccggagccaactggagcgtgaacttccacaggattaacgagaatgagaagtctcccagtcagaaccggaaagccaaggacgccacctcagacaacggcaaagacggcctggcctactctgccctgctcaagaatgagctgctgggtgccggcatcgagaaggtgcaggacccgcagactgaggaccgcaggctgcagccctccacgcctgagaagaagggtctgttcacgtattcccttagcaccaagcgctccagccccgatgacggcaacgatgtgtctccctactccctgtctcccgtcagcaacaagagccagaagctgctccggtccccccggaaacccacccgcaagatctccaagatccccttcaaggtgctggacgcgcccgagctgcaggacgacttctacctcaatctggtggactggtcgtccctcaatgtgctcagcgtggggctaggcacctgcgtgtacctgtggagtgcctgtaccagccaggtgacgcggctctgtgacctctcagtggaaggggactcagtgacctccgtgggctggtctgagcgggggaacctggtggcggtgggcacacacaagggcttcgtgcagatctgggacgcagccgcagggaagaagctgtccatgttggagggccacacggcacgcgtcggggcgctggcctggaatgctgagcagctgtcgtccgggagccgcgaccgcatgatcctgcagagggacatccgcaccccgccactgcagtcggagcggcggctgcagggccaccggcaggaggtgtgcgggctcaagtggtccacagaccaccagctcctcgcctcggggggcaacgacaacaagctgctggtctggaatcactcgagcctgagccccgtgcagcagtacacggagcacctggcggccgtgaaggccatcgcctggtccccacatcagcacgggctgctggcctcggggggcggcacagctgaccgctgtatccgcttctggaacacgctgacaggacaaccactgcagtgtatcgacacgggctcccaagtgtgcaatctggcctggtccaagcacgccaacgagctggtgagcacgcacggctactcacagaaccagatccttgtctggaagtacccctccctgacccaggtggccaagctgaccgggcactcctaccgcgtgctgtacctggcaatgtcccctgatggggaggccatcgtcactggtgctggagacgagaccctgaggttctggaacgtctttagcaaaacccgttcgacaaaggtaaagtgggagtctgtgtctgtgctcaacctcttcaccaggatccggtaa

SEQ ID NO: 24 - protein, 496 aa, Homo sapiens
MDQDYERRLLRQIVIQNENTMPRVTEMRRTLTPASSPVSSPSKHGDRFIPSRAGANWSVNFHRINENEKSPSQNRKAKDATSDNGKDGLAYSALLKNELLGAGIEKVQDPQTEDRRLQPSTPEKKGLFTYSLSTKRSSPDDGNDVSPYSLSPVSNKSQKLLRSPRKPTRKISKIPFKVLDAPELQDDFYLNLVDWSSLNVLSVGLGTCVYLWSACTSQVTRLCDLSVEGDSVTSVGWSERGNLVAVGTHKGFVQIWDAAAGKKLSMLEGHTARVGALAWNAEQLSSGSRDRMILQRDIRTPPLQSERRLQGHRQEVCGLKWSTDHQLLASGGNDNKLLVWNHSSLSPVQQYTEHLAAVKAIAWSPHQHGLLASGGGTADRCIRFWNTLTGQPLQCIDTGSQVCNLAWSKHANELVSTHGYSQNQILVWKYPSLTQVAKLTGHSYRVLYLAMSPDGEAIVTGAGDETLRFWNVFSKTRSTKVKWESVSVLNLFTRIR

SEQ ID NO: 25 - DNA, 1482 bp, Mus musculus
  CDS (1)..(1479)
atggaccaggactatgagcgaaggctcctgcggcagatcatcatccagaatgagaacacagtgccctgtgtttcagagatgcggagaaccctgacaccagccaactccccagtgtcttcacccagcaagcatggtgaccgcttcatcccctcgcgggccggggccaactggagcgtgaacttccacaggatcaatgaaaatgagaagtcccccagccagaaccgcaaagccaaggacgccacctcggacaatggcaaagacggcctggcctactccgcactgctgaagaatgagctgctgggtgccggcatcgagaaggttcaggacccacagacggaggaccggcggctgcagccgtccacaccagagcacaaggggctctttacgtattccctcagcagcaagcgctcgagtccagatgatggcaatgacgtgtccccatactctctgtcccccgttagcaacaaaagtcagaagctgctgcggtcaccacggaagcccacacgcaagatctctaagattcccttcaaggtgctggacgcgccagagcttcaggacgacttctacctcaacttggtggactggtcctccctcaacgtgctcagcgtggggctgggcacctgcgtgtacctgtggagtgcatgcaccagccaggtgacccggctctgtgacctctctgtagaaggggactcagtgacttccgttggctggtctgagcgggggaacttggtcgcagtaggtacacacaagggcttcgtgcagatctgggacgctgctgctgggaagaagctgtccatgctggagggccacacagcacgagtgggggcgctggcctggaatgctgaccagttgtcatctggtagccgtgaccgcatgatcctacagcgggatatccgcacaccacccctgcagtcagagcggcggctgcagggccaccggcaggaagtgtgtggcctaaagtggtccacagaccaccagctgcttgcctcggggggcaatgacaacaagctgctcgtgtggaaccactctagtctaagccctgtgcagcagtatacggagcacctggcagccgtgaaggctattgcctggtccccacaccagcatggactgctggcatctggtggtggcacggctgaccgctgcatccgattctggaacactctgacaggtcagccactgcagtgcattgacacaggctcacaagtgtgcaacctggcctggtccaagcacgccaatgagctggtgagcacacatggctactcacagaaccagatcctcgtgtggaagtacccgtcccttacgcaggtggccaagctcactggccactcatatcgtgtcctctacctggccatgtcccctgatggggaggccatagtcaccggagctggagatgagaccctgaggttctggaatgtcttcagcaaaacacgctctacaaaggaatctgtgtctgtgctcaacctcttcacccggatccgatag

SEQ ID NO: 26 - protein, 493 aa, Mus musculus
MDQDYERRLLRQIIIQNENTVPCVSEMRRTLTPANSPVSSPSKHGDRFIPSRAGANWSVNFHRINENEKSPSQNRKAKDATSDNGKDGLAYSALLKNELLGAGIEKVQDPQTEDRRLQPSTPEHKGLFTYSLSSKRSSPDDGNDVSPYSLSPVSNKSQKLLRSPRKPTRKISKIPFKVLDAPELQDDFYLNLVDWSSLNVLSVGLGTCVYLWSACTSQVTRLCDLSVEGDSVTSVGWSERGNLVAVGTHKGFVQIWDAAAGKKLSMLEGHTARVGALAWNADQLSSGSRDRMILQRDIRTPPLQSERRLQGHRQEVCGLKWSTDHQLLASGGNDNKLLVWNHSSLSPVQQYTEHLAAVKAIAWSPHQHGLLASGGGTADRCIRFWNTLTGQPLQCIDTGSQVCNLAWSKHANELVSTHGYSQNQILVWKYPSLTQVAKLTGHSYRVLYLAMSPDGEAIVTGAGDETLRFWNVFSKTRSTKESVSVLNLFTRIR

SEQ ID NO: 27 - DNA, 1428 bp, Medicago sativa
  CDS (1)..(1425)
atggacggaaccggtaatcgaaatccaccaccgacttccaccgtcagagacaattctccaccgcctgagccatcaccggagagtctccgtcatgtaagccgtatgatcaacagcaaccattacacctcaccttctcgaacaatctactccgataggttcattccgagtagatctgcttcgaaattcgctttgtttgatatcaatactccgacagaaggacgcgatgatagttccagcgcttatacgactcttctgagaacggcgttgtttggaccggatgttgccggtccggttacgccggaaaaaaccgactcgccgtcgatgacattgccgaataggaatatttttaggtataagacggagacgagacagtccatgcactcgctttcgccgtttatggatgatgattttgttcctggtgttaatcatagtccggttaaggctcctaggaaggttcctcgatcgccttataaggttttggatgcacctgctttgcaagatgatttttatctgaatctggtagattggtcttcacacaatgtgttggctgttggtttgggtaactgtgtctatctctggaatgcttgtagcagcaaggtaactaaattatgtgatttgggggttgatgattgtgtttgttctgttggttgggctcaacgtggtactcatcttgctgttggaactaacaatggtaaagttcagatttgggatgcagcaagatgcaagaagataagatcaatggagggccatcggttacgtgtcggggccttggcctggagttcatctcttttgtcttctggtggacgggataagaatatttatcaacgagatatacgcacacaagaagattttgttagtaaactgtcaggacacaaatcagaggtttgtggactgaagtggtcatatgataaccgtgagttggcatctggaggaaatgacaacaaattgtttgtttggaatcaacactcaacccagcctgtcctcaagtactgtgagcacacagcagctgttaaagctattgcatggtctcctcatcttcatggacttcttgcatctggaggaggaactgcagatagatgtattcgtttttggaatacaaccacaaactcacaccttagctgtatggacactggaagtcaggtttgcaatcttgtctggtccaaaaatgtcaacgaactagtaagcacacatgggtactcccagaaccagattattgtttggagataccccactatgtcaaagctggcgactcttaccggccatacctatagggttctctatcttgccatctctccagatggacagactattgtaactggagctggagatgaaacgcttaggttctggaatgttttcccttcccctaaatcacagaatactgaaagtgaaatcggagcattatctcttggaagaactactatcaggtga

SEQ ID NO: 28 - protein, 475 aa, Medicago sativa
MDGTGNRNPPPTSTVRDNSPPPEPSPESLRHVSRMINSNHYTSPSRTIYSDRFIPSRSASKFALFDINTPTEGRDDSSSAYTTLLRTALFGPDVAGPVTPEKTDSPSMTLPNRNIFRYKTETRQSMHSLSPFMDDDFVPGVNHSPVKAPRKVPRSPYKVLDAPALQDDFYLNLVDWSSHNVLAVGLGNCVYLWNACSSKVTKLCDLGVDDCVCSVGWAQRGTHLAVGTNNGKVQIWDAARCKKIRSMEGHRLRVGALAWSSSLLSSGGRDKNIYQRDIRTQEDFVSKLSGHKSEVCGLKWSYDNRELASGGNDNKLFVWNQHSTQPVLKYCEHTAAVKAIAWSPHLHGLLASGGGTADRCIRFWNTTTNSHLSCMDTGSQVCNLVWSKNVNELVSTHGYSQNQIIVWRYPTMSKLATLTGHTYRVLYLAISPDGQTIVTGAGDETLRFWNVFPSPKSQNTESEIGALSLGRTTIR

SEQ ID NO: 29 - DNA, 1410 bp, Oryza sativa
  CDS (1)..(1407)
atggacgcaggctcccactcgatctcgtcggagaagagccacggactcgcgccgcggccgccgctccaggaggccggctcccgcccgtacatgccatcgctgagcactgcctcgcgcaacccgtcggccaagtgctacggggataggttcataccggacaggtcggcgatggacatggacatggcgcactacctgctcacggagcccaagaaggacaaggagaacgcggcggcgtccccgtccaaggaggtgtaccggaggctgctcgccgagaagctgctcaacaaccggacacggatcctcgccttccggaacaagccaccggagcccgagaacgtctctgccgcggatactgcttctacccaccaggccaagccggccaagcagcggcgctacattccccagtctgccgagaggactctggatgcaccggacctcgtcgacgattactatctcaacctgctggactggggaagcaagaacgtgctgtccattgcgctgggcgacacggtgtacctgtgggacgcctcgagcggatccacatctgagctcgtgaccgtggacgaggacagcggcccgatcaccagcgtcagctgggctcctgacggccagcatgtcgccgttggcctcaactcttccgacatccagctctgggacaccagctccaaccgactgcttagaactctgagaggtgtgcacgaatcaagagtcggttcactcgcatggaacaacaacatcctgaccactggcggtatggacggcaacatcgtgaacaatgacgtaagaatcaggaaccatgtcgtgcagacataccaggggcacagccaggaggtgtgcgggctcaagtggtctggttcagggcagcagctggccagtggtggcaacgacaaccttcttcacatttgggatgtgtccatggcatcgtccgtcccatctgcaggccgcaaccagtggctgcacaggctcgaggatcacacggccgctgtgaaagcgcttgcatggtgcccattccagagcaacctgcttgcaactggaggtggtggtagcgaccggtgcatcaagttctggaacacacacaccggtgcatgcttgaactctgtcgataccggatcgcaggtgtgcgctcttctatggaacaaaaatgagagagagcttctgagttcacacggattcacgcagaaccagctgacattgtggaagtacccgtcgatggttaagatggctgaactcactggccatacttcccgtgtccttttcatggctcagagtcctgatggttgcacagtagcatcggctgctgcagatgagaccctgcggttctggaatgtgtttggtagtcctgaagcccccaagcctgcagccaaggcttcccacactgggatgttcaacagcttcaaccatctcagataa

SEQ ID NO: 30 - protein, 469 aa, Oryza sativa
MDAGSHSISSEKSHGLAPRPPLQEAGSRPYMPSLSTASRNPSAKCYGDRFIPDRSAMDMDMAHYLLTEPKKDKENAAASPSKEVYRRLLAEKLLNNRTRILAFRNKPPEPENVSAADTASTHQAKPAKQRRYIPQSAERTLDAPDLVDDYYLNLLDWGSKNVLSIALGDTVYLWDASSGSTSELVTVDEDSGPITSVSWAPDGQHVAVGLNSSDIQLWDTSSNRLLRTLRGVHESRVGSLAWNNNILTTGGMDGNIVNNDVRIRNHVVQTYQGHSQEVCGLKWSGSGQQLASGGNDNLLHIWDVSMASSVPSAGRNQWLHRLEDHTAAVKALAWCPFQSNLLATGGGGSDRCIKFWNTHTGACLNSVDTGSQVCALLWNKNERELLSSHGFTQNQLTLWKYPSMVKMAELTGHTSRVLFMAQSPDGCTVASAAADETLRFWNVFGSPEAPKPAAKASHTGMFNSFNHLR

SEQ ID NO: 31 - protein, 525 aa, Artificial Sequence
  consensus sequence obtained by aligning amino acid sequences of ccs52/FZRs from Arabidopsis, maize, sorghum, Homo sapiens, rice, soybean, mouse, alfalfa, Drosophila, and Xenopus. See Figure 2.
XXXXDXXXXXXXXXXXXXXXXXXXXXXXXXXSXXXXSXVXRXIXSXXXXSPSXXXXXKSXYSDRFIPSRSGSXXXXNFXLFXLXXXXXXXXXXXXXXXXXXXXSXXAYSXLLKXXLFGXXXXXXXXXXXXXXXXXXXXXXXXRNIFRYKTETRXXXXXSXXSXSPXXXXXXXXXSXXVXXSXXPXKXPRKIPRSPFKVLDAPALQDDFYLNLVDWSSNNVLAVGLGCVYLWACSSKVTKLCDLGVDDSVCSVGWAXRGTHLAVGTXXGXVQIWDASXXKKIRTLEGXHXXRVGALAWNSSLLSSGXRDKXILQRDIRXQEDXVSXKLXGHKSEVCGLKWSXDNRELASGGNDNRLXVWNQXSXXXXXXXXXXQPVLKYXEHTAAVKAIAWSPHXHGLLASGGGTADRCIRFWNTTTGXXLNCIDTGSQVCNLVWSKNVNELVSTHGYSQNQIIVWKYPSMSKLATLTGHTYRVLYLAISPDGQTIVTGAGDETLRFWNVFPSPKSQXXXSEXGASXXGRTXIRXX